(19) United States
(12) Patent Application Publication
(10) Pub. No.: US 2026/0347325 A1
(43) Pub. Date: Nov. 5, 2026

(54) DIPHENYLPYRAZOLE COMPOUNDS AND THEIR USE

(71) Applicants: INSTITUT NATIONAL DE LA SANTÉ ET DE LA RECHERCHE MÉDICALE, Paris (FR); UNIVERSITE DE LILLE, Lille (FR); CENTRE HOSPITALIER UNIVERSITAIRE DE LILLE, Lille (FR)

(72) Inventors: Patricia MELNYK, Lille Cedex (FR); Nicolas SERGEANT, Lille (FR); Pascal CARATO, Poitiers (FR); Christophe MESANGEAU, Lille Cedex (FR)

(21) Appl. No.: 19/117,452

(22) PCT Filed: Oct. 12, 2023
(86) PCT No.: PCT/EP2023/078422

§ 371 Date: Apr. 1, 2025

(30) Foreign Application Priority Data
Oct. 12, 2022 (EP) 22306550.9

(51) Int. Cl. C07D 231/22; A61K 31/415

(52) U.S. Cl. CPC C07D 231/22; A61K 31/415

(57) ABSTRACT

The inventors have now succeeded in developing novel compounds comprising a diphenylpyrazole scaffold bearing amino side chains. These compounds have the advantage of repressing the production of Aβ1-x and modulating the ratio of autophagy markers with higher efficacy than chloroquine and compounds of the prior art, in particular compounds of the prior art, The present invention is thus directed to compounds of Formula (I) including their pharmaceutically acceptable salts and solvates which have the ability to repress the production of Aβ1-x peptides and to modulate the ratio of autophagy markers, and which are useful as therapeutic compounds, particularly in the treatment and/or prevention of diseases involving the formation of amyloid plaques and/or in which dysfunction of the APP metabolism occurs.

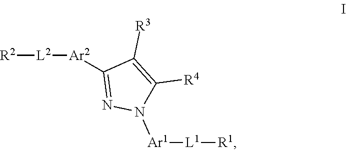

The present invention relates to novel diphenylpyrazole compounds including their pharmaceutically acceptable salts and solvates which have the ability to repress the production of Aβ₁₋ₓ peptides and to modulate the ratio of autophagy markers, and which are useful as therapeutic compounds, particularly in the treatment and/or prevention of diseases involving the formation of amyloid plaques and/or in which dysfunction of the APP metabolism occurs.

### BACKGROUND OF THE INVENTION

Alzheimer's disease (AD), the most frequent form of dementia worldwide, combines a slow decline of cognitive and behavioral disabilities and progressive neuropathological processes. The latter include neurofibrillary tangles (NFTs) and amyloid deposits together with astrogliosis, neuroinflammation, and neuronal death (A. Serrano-Pozo et al., Cold Spring Harb. Perspect. Med. 2011, 1, 1-23). Intraneuronal accumulation and aggregation of abnormally modified isoforms of the microtubule-associated Tau proteins are the components of NFTs. Parenchymal amyloid deposits are composed of amyloid-O (A3) peptides originating from complex sequential proteolytic cleavages of a precursor protein, namely the amyloid precursor protein (APP) (B. De Strooper, Physiol. Rev. 2010, 90, 465-494). The non-amyloidogenic pathway, initiated by the α-secretase cleavage of APP, is opposed to the amyloidogenic pathway resulting from the primary cleavage by the β-secretase occurring at the first amino acid of Aβ peptide sequence (R. J. Andrew et al., J. Biol. Chem. 2016, 291, 19235-19244; H. S. Nhan et al., Acta Neuropathol. 2015, 129, 1-19). Both secretase-mediated steps shed soluble ectodomains of APP (sAPPα and sAPPβ) and membrane-bound carboxyl-terminal fragments (APP-CTFs), known as αCTF and βCTF. The latter is further cleaved by the γ-secretase to give rise to p3 and Aβ peptides, respectively, along with the APP intracellular domain (AICD). Along the secretory pathway, APP can also be cleaved by the β-secretase at β′-site at position 11 of the Aβ peptide sequence suggested to be protective (J. T. Huse et al., J. Biol. Chem. 2002, 277(18), 16278-84; A. K. McKendell et al., Biosensors (Basel) 2022, 12(8), 663; A. Kimura et al., J. Biol. Chem. 2016, 291(46), 24041-24053; Y. Deng et al., Eur. J. Neurosci. 2013, 37(12), 1962-9). The definite diagnosis relies on these pathological processes, which are therefore part of AD pathophysiology and should be considered as a whole for therapeutic development.

AD treatment remains symptomatic, and currently, disease-modifying treatment remains ill-defined except for Aducanumab immunotherapy, which reduces the amyloid load and Tau PET imaging in clinical rails (G. D. Rabinovici, N. Engl. J. Med. 2021, 385(9), 771-774). The definite diagnosis relies on these pathological processes, which are therefore all related to AD pathophysiology. Therefore, alternative disease-modifying therapeutic options are urgently needed, and over the past few years, much effort has been dedicated to the development of such disease-modifying drugs (J. Cummings et al., Alzheimer's Dement.: Transl. Res. Clin. Interv. 2022, 8(1), e12295). One such strategy would be the development of drugs that efficiently modify both amyloid and Tau pathological processes.

Redirection of APP processing can be achieved either by blocking the amyloidogenic pathway or by promoting the non-amyloidogenic pathway. The results of these activities would be a decrease in Aβ secretion and an increase in αCTF. Chloroquine (CQ) was previously shown to inhibit Aβ production, whereas levels of other APP metabolites such as APP-CTFs and AICD are maintained and even increased, whereas the γ-secretase cleavage of Notch remains unmodified precluding the contribution of the γ-secretase in this process (V. Vingtdeux et al., Neurobiol. Dis. 2007, 25, 686-696; V. Vingtdeux et al., J. Biol. Chem. 2007, 282, 18197-18205).

WO 2006/051489 A1 and P. Melnyk et al., ACS Chem. Neurosci. 2015, 6, 559-569 disclose the use of 1,4-bis(3-aminoalkyl)piperazine derivatives for the treatment of neurodegenerative diseases, wherein said derivatives may be used to rectify the metabolism of the amyloid protein precursor (APP).

WO 2011/073322 A1 and M. Gay et al., Bioorg. Med. Chem. 2018, 26, 2151-2164 discloses the use of 7-chloro-quinolin-4-amine compounds for the prevention or treatment of diseases involving the formation of amyloid plaques and/or in which dysfunction of the APP metabolism occurs.

WO 2020/011848 A1 and M. Gay et al., Eur. J. Med. Chem. 2018, 159, 104-125 disclose the use of polyamino biaryl compounds for the prevention or treatment of diseases involving formation of amyloid plaques and/or in which dysfunction of the APP metabolism occurs.

However, there is still a need for new compounds having the ability to repress the Aβ₁₋ₓ peptides production, and to modulate the ratio of autophagy markers, and that are of therapeutic value for the treatment and/or prevention of diseases involving the formation of amyloid plaques and/or in which dysfunction of the APP metabolism occurs.

### SUMMARY OF THE INVENTION

The inventors have now succeeded in developing novel compounds comprising a diphenylpyrazole scaffold bearing amino side chains. These compounds have the advantage of repressing the production of Aβ₁₋ₓ, in particular with low repression of Aβ_{x-38/40/42} released concentrations, and modulating the ratio of autophagy markers with higher efficacy than chloroquine and compounds of the prior art, in particular compounds of WO 2006/051489 A1 and WO 2020/011848 A1.

The invention therefore relates to compounds of general Formula I, their pharmaceutically acceptable salts and solvates, as well as methods of use of such compounds or compositions comprising such compounds to repress the Aβ₁₋ₓ peptides production and modulate the ratio of autophagy markers.

In a general aspect, the invention provides compounds of general Formula I:

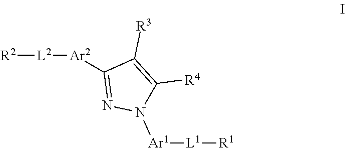

and pharmaceutically acceptable salts and solvates thereof,
wherein
Ar¹ is phenyl optionally substituted by one or more substituent(s) selected from the group consisting of halo, C1-C3-alkyl, C1-C3-haloalkyl and C1-C3-alkoxy;
L¹ is C1-C4-alkylene optionally substituted by one or more substituent(s) selected from the group consisting of halo and C1-C3-alkyl;
R¹ is selected from the group consisting of —OH, —N(C1-C6-alkyl)₂

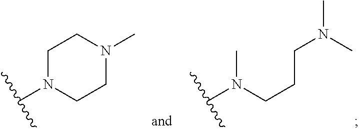

Ar² is phenyl optionally substituted by one or more substituent(s) selected from the group consisting of halo, C1-C3-alkyl, C1-C3-haloalkyl and C1-C3-alkoxy;
L² is C1-C4-alkylene optionally substituted by one or more substituent(s) selected from the group consisting of halo and C1-C3-alkyl;
R² is selected from the group consisting of —N(C1-C6-alkyl)₂,

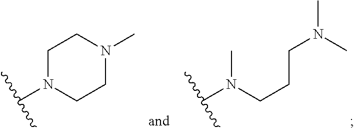

R³ is C1-C4-alkyl or —(CH₂)ₙ—NMe₂ wherein n is an integer from 1 to 3; and
R⁴ is H, halo or C1-C4-alkyl.

In another aspect, the present invention provides a pharmaceutical composition comprising at least one compound according to the invention, or a pharmaceutically acceptable salt or solvate thereof, and at least one pharmaceutically acceptable carrier, diluent, excipient, and/or adjuvant.

The invention further provides the use of a compound according to the invention, or a pharmaceutically acceptable salt or solvate thereof, as a medicament.

The invention also relates to the compound of Formula I for use in the treatment and/or prevention of diseases involving formation of amyloid plaques and/or in which dysfunction of the APP metabolism occurs.

### DETAILED DESCRIPTION OF THE INVENTION

As detailed above, the invention relates to compounds of Formula I, as well as their pharmaceutically acceptable salts and solvates.

Particular compounds of Formula I and pharmaceutically acceptable salts and solvates thereof are those wherein one or more of Ar¹, L¹, R¹, Ar², L², R², R³ and R⁴ are defined as follows:
Ar¹ is phenyl optionally substituted by one or more substituent(s) selected from the group consisting of halo, C1-C3-alkyl, C1-C3-haloalky,l, and C1-C3-alkoxy; in particular Ar¹ is phenyl optionally substituted by one or more substituent(s) selected from the group consisting of halo, C1-C3-alkyl, and C1-C3-haloalkyl; more particularly Ar¹ is phenyl optionally substituted by one or more substituent(s) selected from the group consisting of halo and C1-C3-alkyl; still more particularly Ar¹ is phenyl optionally substituted by one or more substituent(s) selected from the group consisting of F, C1, and C1-C3-alkyl; even more particularly Ar¹ is phenyl;
L¹ is C1-C4-alkylene optionally substituted by one or more substituent(s) selected from the group consisting of halo and C1-C3-alkyl; in particular, L¹ is C1-C3-alkylene optionally substituted by one or more substituent(s) selected from the group consisting of F, Cl and C1-C3-alkyl; more particularly L¹ is C1-C3-alkylene; still more particularly L¹ is CH₂;
R¹ is selected from the group consisting of —OH, —N(C1-C6-alkyl)₂,

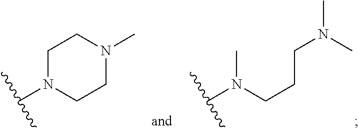

in particular R¹ is selected from the group consisting of —OH, —N(C1-C4-alkyl)₂

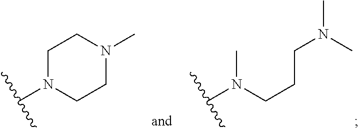

more particularly R¹ is selected from the group consisting of —OH, —N(C1-C2-alkyl)₂,

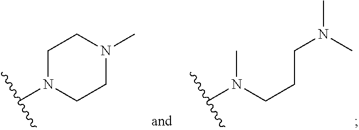

even-more particularly R¹ is selected from the group consisting of —OH, —NMe₂,

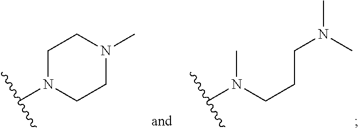

in a particular example, R¹ is selected from the group consisting of —NMe₂, and

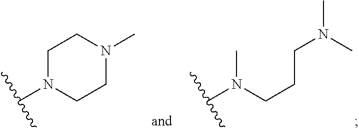

Ar² is phenyl optionally substituted by one or more substituent(s) selected from the group consisting of halo, C1-C3-alkyl, C1-C3-haloalkyl and C1-C3-alkoxy; in particular Ar² is phenyl optionally substituted by one or more substituent(s) selected from the group consisting of halo, C1-C3-alkyl and C1-C3-haloalkyl; more particularly Ar² is phenyl optionally substituted by one or more substituent(s) selected from the group consisting of halo and C1-C3-alkyl; still more particularly Ar² is phenyl optionally substituted by one or more substituent(s) selected from the group consisting of F, Cl and C1-C3-alkyl; even more particularly Ar² is phenyl;
L² is C1-C4-alkylene optionally substituted by one or more substituent(s) selected from the group consisting of halo and C1-C3-alkyl; in particular L² is C1-C3-alkylene optionally substituted by one or more substituent(s) selected from the group consisting of F, Cl and C1-C3-alkyl; more particularly L² is C1-C3-alkylene;
R² is selected from the group consisting of —N(C1-C6-alkyl)₂,

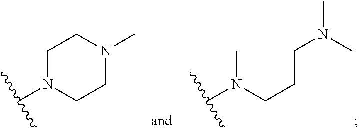

in particular R² is selected from the group consisting of —N(C1-C4-alkyl)₂,

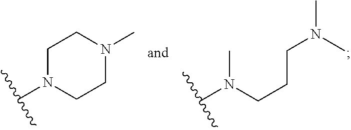

more particularly R² is selected from the group consisting of —N(C1-C2-alkyl)₂,

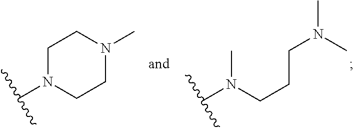

even more particularly R² is selected from the group consisting of —NMe₂,

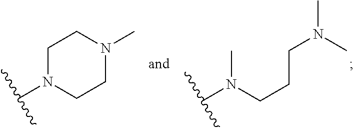

R³ is C1-C4-alkyl or —(CH₂)ₙ—NMe₂ wherein n is an integer from 1 to 3; in particular R³ is C1-C3-alkyl or —(CH₂)ₙ—NMe₂ wherein n is an integer from 1 to 3, more particularly R³ is C1-C2-alkyl or —(CH₂)ₙ—NMe₂ wherein n is an integer from 1 to 3; still more particularly R³ is methyl or —(CH₂)ₙ—NMe₂ wherein n is an integer from 1 to 3; and
R⁴ is H, halo or C1-C4-alkyl; in particular R⁴ is H, F, Cl or C1-C3-alkyl; more particularly R⁴ is H, F or C1-C3-alkyl; still more particularly R⁴ is H or C1-C3-alkyl; even more particularly R⁴ is H.

In one embodiment, the compounds of Formula I are those wherein Ar¹ is phenyl.

In one embodiment, the compounds of Formula I are those wherein Ar² is phenyl.

In one embodiment, the compounds of Formula I are those wherein Ar¹ and Ar² are phenyl.

In one embodiment, the compounds of Formula I are those wherein L¹ is CH₂.

In one embodiment, the compounds of Formula I are those wherein L² is C1-C3-alkylene.

In one embodiment, the compounds of Formula I are those wherein L¹ is CH₂ and L² is C1-C3-alkylene.

In one embodiment, the compounds of Formula I are those wherein R³ is C1-C4-alkyl; in particular R³ is C1-C3-alkyl; more particularly R³ is C1-C2-alkyl; still more particularly R³ is methyl.

In one embodiment, the compounds of Formula I are those wherein R³ is —(CH₂)ₙ—NMe₂ wherein n is an integer from 1 to 3. In one particular example, the compounds of Formula I are those wherein R³ is —(CH₂)—NMe₂. In another particular example, the compounds of Formula I are those wherein R³ is —(CH₂)₃—NMe₂.

In one embodiment, the compounds of Formula I are those wherein R⁴ is H.

In one embodiment, the compounds of Formula I are those wherein Ar¹ and Ar² are phenyl and L¹ is CH₂.

In one embodiment, the compounds of Formula I are those wherein Ar¹ and Ar² are phenyl and L² is C1-C3-alkylene.

In one embodiment, the compounds of Formula I are those wherein Ar¹ and Ar² are phenyl, L¹ is CH₂ and L² is C1-C3-alkylene.

In one embodiment, the compounds of Formula I are those wherein Ar¹ and Ar² are phenyl and R⁴ is H.

In one embodiment, the compounds of Formula I are those wherein Ar¹ and Ar² are phenyl, L¹ is CH₂ and R⁴ is H.

In one embodiment, the compounds of Formula I are those wherein Ar¹ and Ar² are phenyl, L² is C1-C3-alkylene and R⁴ is H.

In one embodiment, the compounds of Formula I are those wherein Ar¹ and Ar² are phenyl, L¹ is CH₂, L² is C1-C3-alkylene and R⁴ is H.

In one embodiment, the compounds of Formula I are those of Formula II:

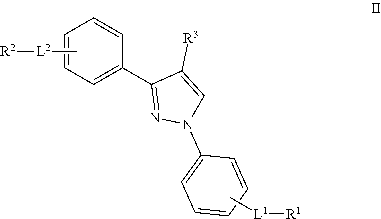

and pharmaceutically acceptable salts and solvates thereof,
wherein
L¹, R¹, L², R² and R³ are as defined above with respect to Formula I and any of its embodiments.

In one embodiment, the compounds of Formula I are those of Formula III:

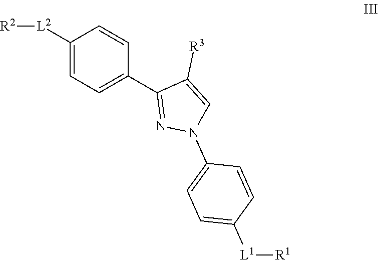

and pharmaceutically acceptable salts and solvates thereof,
wherein
L¹, R¹, L², R² and R³ are as defined above with respect to Formula I and any of its embodiments.

In one embodiment, the compounds of Formula I are those of Formula IV:

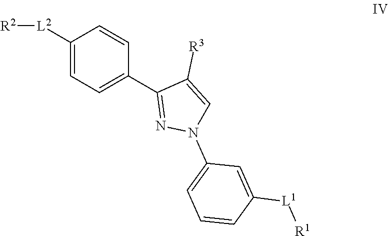

and pharmaceutically acceptable salts and solvates thereof,
wherein
L¹, R¹, L², R² and R³ are as defined above with respect to Formula I and any of its embodiments.

In one embodiment, the compounds of Formula I are those of Formula V:

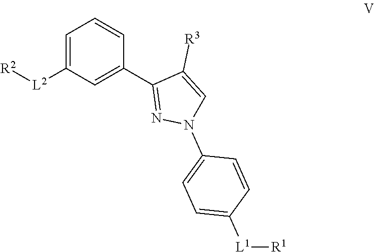

and pharmaceutically acceptable salts and solvates thereof,
wherein
L¹, R¹, L², R² and R³ are as defined above with respect to Formula I and any of its embodiments.

In one embodiment, the compounds of Formula I are those of Formula VI:

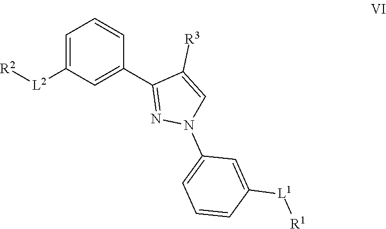

and pharmaceutically acceptable salts and solvates thereof,
wherein
L¹, R¹, L², R² and R³ are as defined above with respect to Formula I and any of its embodiments.

Particularly preferred compounds of the invention are those listed in Table 1 hereafter:

TABLE 1

| Structure | Name |
|---|---|
| 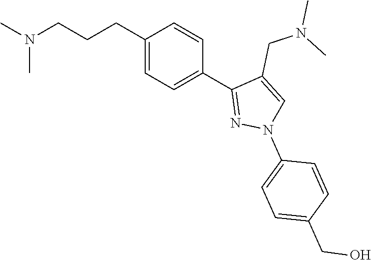 [SMILES: CN(C)CCCc1ccc(-c2nn(-c3ccc(CO)cc3)cc2CN(C)C)cc1] 16 | [4-[4-(dimethylamino- methyl)-3-[4-[3- (dimethylamino)propyl] phenyl]-1H-pyrazol- 1-yl]phenyl]methanol |
| 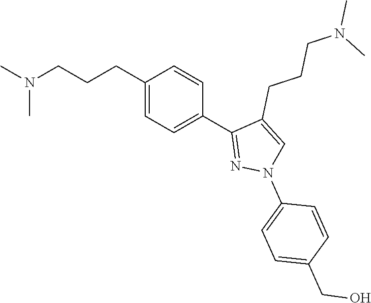 17 | [4-[4-[3-(dimethylamino) propyl]-3-[4-[3- (dimethylamino)propyl] phenyl]-1H-pyrazol- 1-yl]phenyl]methanol |
| 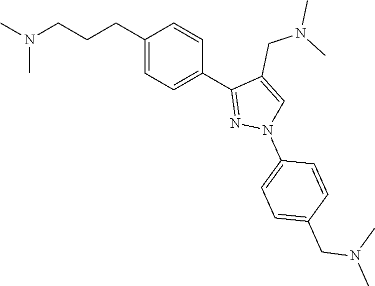 24 | 3-[4-[4-(dimethyl- aminomethyl)-1-[4- (dimethylaminomethyl) phenyl]-1H-pyrazol- 3-yl]phenyl]-N,N-dimethyl- propan-1-amine |
| 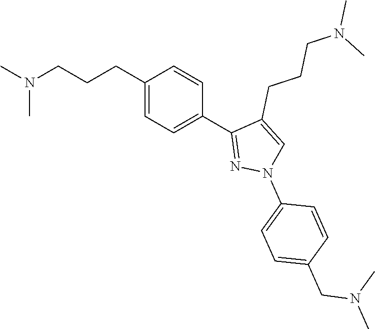 25 | 3-[4-[1-[4-(dimethylamino- methyl)phenyl]-4- [3-(dimethylamino)propyl]- 1H-pyrazol-3- yl]phenyl]-N,N-dimethyl- propan-1-amine |
| 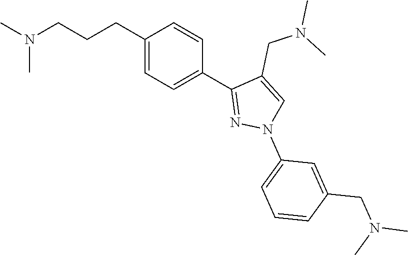 26 | 3-[4-[4-(dimethyl- aminomethyl)-1-[3- (dimethylaminomethyl) phenyl]-1H-pyrazol- 3-yl]phenyl]-N,N-dimethyl- propan-1-amine |
| 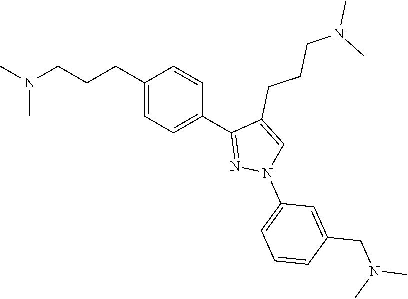 27 | 3-[4-[1-[3-(dimethyl- aminomethyl)phenyl]-4- [3-(dimethylamino)propyl]- 1H-pyrazol-3- yl]phenyl]-N,N-dimethyl- propan-1-amine |
| 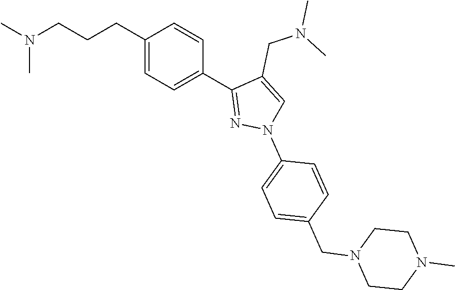 28 | 3-[4-[4-(dimethyl- aminomethyl)-1-[4-[(4- methylpiperazin-1-yl) methyl]phenyl]-1H- pyrazol-3-yl]phenyl]-N,N- dimethyl-propan-1- amine |
| 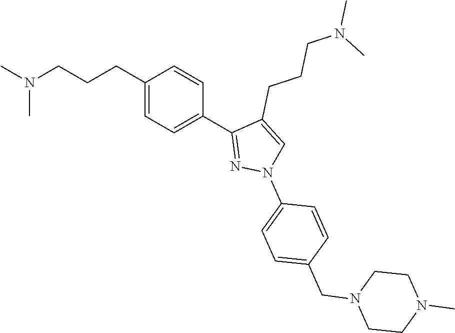 29 | 3-[4-[4-[3-(dimethylamino) propyl]-1-[4-[(4- methylpiperazin-1-yl) methyl]phenyl]-1H- pyrazol-3-yl]phenyl]-N,N- dimethyl-propan-1- amine |
| 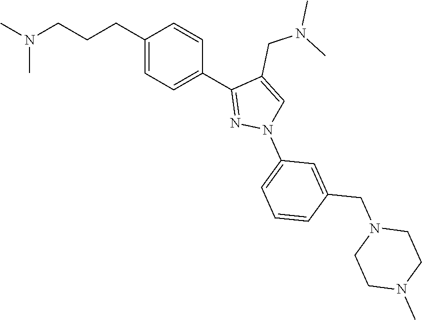 [SMILES: CN(C)CCCc1ccc(-c2nn(-c3cccc(CN4CCN(C)CC4)c3)cc2CN(C)C)cc1] 30 | 3-[4-[4-(dimethylamino- methyl)-1-[3-[(4- methylpiperazin-1-yl) methyl]phenyl]-1H- pyrazol-3-yl]phenyl]-N,N- dimethyl-propan-1- amine |
| 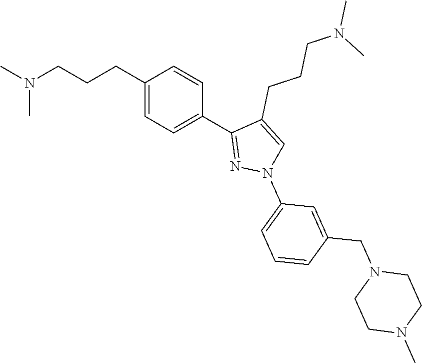 [SMILES: CN(C)CCCc1ccc(-c2nn(-c3cccc(CN4CCN(C)CC4)c3)cc2CCCN(C)C)cc1] 31 | 3-[4-[4-[3-(dimethylamino) propyl]-1-[3-[(4- methylpiperazin-1-yl) methyl]phenyl]-1H- pyrazol-3-yl]phenyl]-N,N- dimethyl-propan-1- amine |
| 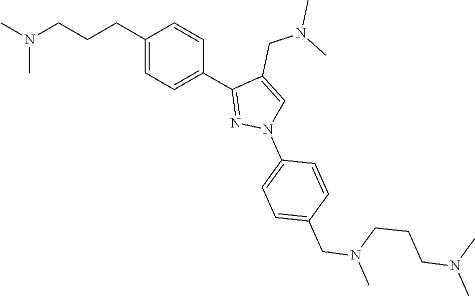 32 | N′-[[4-[4-(dimethyl- aminomethyl)-3-[4-[3- (dimethylamino)propyl] phenyl]-1H-pyrazol- 1-yl]phenyl]methyl]- N,N,N′-trimethyl- propane-1,3-diamine |
| 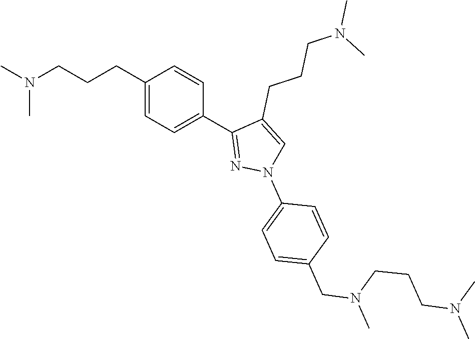 33 | N′-[[4-[4-[3-(dimethylamino) propyl]-3-[4-[3- (dimethylamino)propyl] phenyl]-1Hpyrazol-1- yl]phenyl]methyl]-N,N,N′- trimethyl-propane- 1,3-diamine |
| 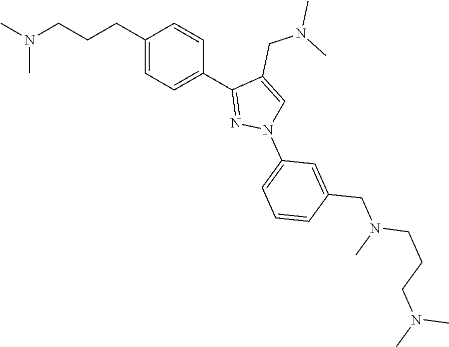 34 | N′-[[3-[4-(dimethyl- aminomethyl)-3-[4-[3- (dimethylamino)propyl] phenyl]-1H-pyrazol- 1-yl]phenyl]methyl]- N,N,N′-trimethyl- propane-1,3-diamine |
| 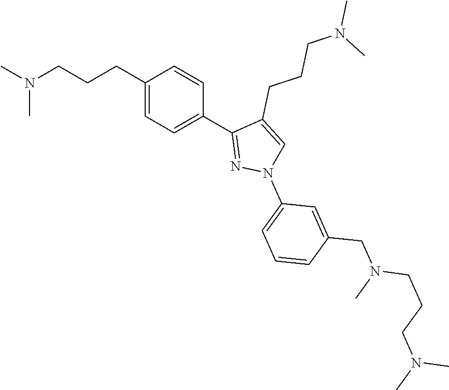 35 | N′-[[3-[4-[3-(dimethyl- amino)propyl]-3-[4-[3- (dimethylamino)propyl] phenyl]-1H-pyrazol- 1-yl]phenyl]methyl]- N,N,N′-trimethyl- propane-1,3-diamine |
| 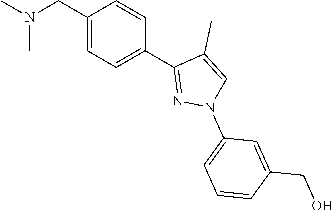 [SMILES: Cc1cn(-c2cccc(CO)c2)nc1-c1ccc(CN(C)C)cc1] 48 | [3-[3-[4-(dimethylamino- methyl)phenyl]-4- methyl-1H-pyrazol-1- yl]phenyl]methanol |
| 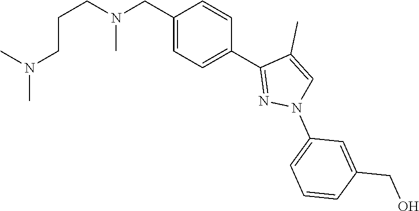 [SMILES: Cc1cn(-c2cccc(CO)c2)nc1-c1ccc(CN(C)CCCN(C)C)cc1] 57 | (3-{3-[4-({[3-(dimethyl- amino)propyl] (methyl)amino}methyl) phenyl]-4-methyl-1H- pyrazol-1-yl} phenyl)methanol |
| 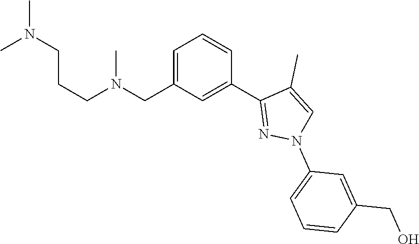 [SMILES: Cc1cn(-c2cccc(CO)c2)nc1-c1cccc(CN(C)CCCN(C)C)c1] 58 | [3-[3-[3-[[(3- (dimethylamino)propyl) (methyl)amino]methyl] phenyl]-4-methyl- 1H-pyrazol-1- yl]phenyl]methanol |
| 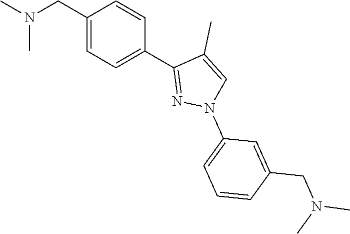 61 | {[4-(1-{3-[(dimethylamino) methyl]phenyl}- 4-methyl-1H- pyrazol-3- yl)phenyl]methyl} dimethylamine |
| 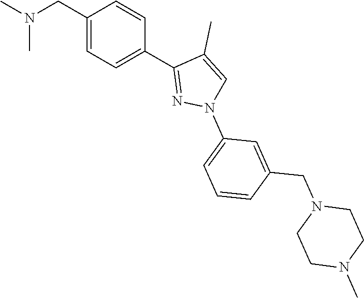 [SMILES: Cc1cn(-c2cccc(CN3CCN(C)CC3)c2)nc1-c1ccc(CN(C)C)cc1] 62 | dimethyl({[4-(4- methyl-1-{3-[(4- methylpiperazin-1-yl) methyl]phenyl}-1H- pyrazol-3-yl)phenyl] methyl})amine |
| 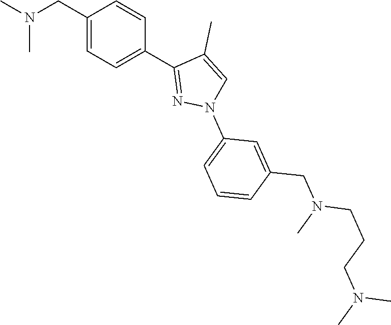 63 | [(4-{1-[3-({[3- (dimethylamino)propyl] (methyl)amino}methyl) phenyl]-4-methyl- 1H-pyrazol-3- yl}phenyl)methyl] dimethylamine |
| 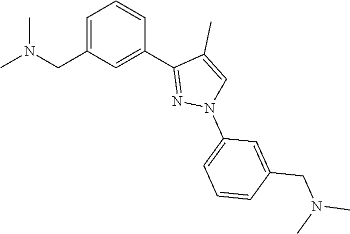 64 | 1-[3-[1-[3-(dimethyl- aminomethyl)phenyl]-4- methyl-1H-pyrazol- 3-yl]phenyl]-N,N- dimethylmethanamine |
| 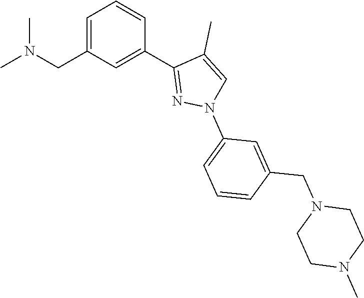 [SMILES: Cc1cn(-c2cccc(CN3CCN(C)CC3)c2)nc1-c1cccc(CN(C)C)c1] 65 | N,N-dimethyl-1-[3- [4-methyl-1-[3-[(4- methylpiperazin-1-yl) methyl]phenyl]-1H- pyrazol-3-yl]phenyl] methanamine |
| 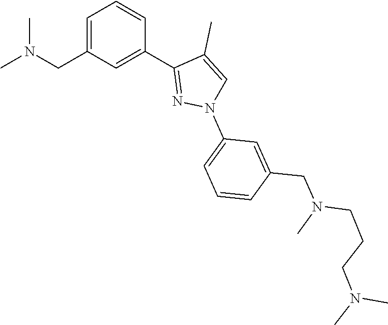 66 | N′-[[3-[3-[3-(dimethyl- aminomethyl)phenyl]- 4-methyl-1H-pyrazol-1- yl]phenyl]methyl]- N,N,N′-trimethyl- propane-1,3-diamine |
| 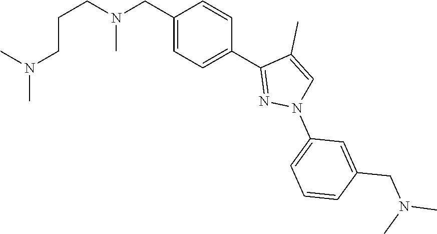 67 | {[4-(1-{3-[(dimethylamino) methyl]phenyl}- 4-methyl-1H-pyrazol-3-yl) phenyl]methyl}-[3- (dimethylamino) propyl]methylamine |
| 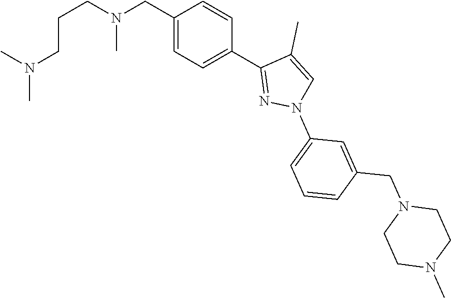 [SMILES: Cc1cn(-c2cccc(CN3CCN(C)CC3)c2)nc1-c1ccc(CN(C)CCCN(C)C)cc1] 68 | [3-(dimethylamino) propyl](methyl){[4-(4- methyl-1-{3-[(4- methylpiperazin-1- yl)methyl]phenyl}- 1H-pyrazol-3- yl)phenyl]methyl}amine |
| 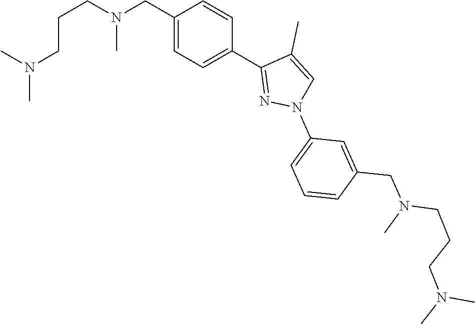 69 | [3-(dimethylamino)propyl] [(4-{1-[3-({[3- (dimethylamino)propyl] (methyl)amino}methyl) phenyl]-4-methyl- 1H-pyrazol-3- yl}phenyl)methyl] methylamine |
| 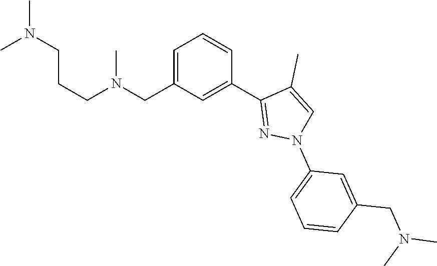 70 | N′-[[3-[1-[3-(dimethyl- aminomethyl)phenyl]- 4-methyl-1H-pyrazol-3- yl]phenyl]methyl]- N,N,N′-trimethyl- propane-1,3-diamine |
| 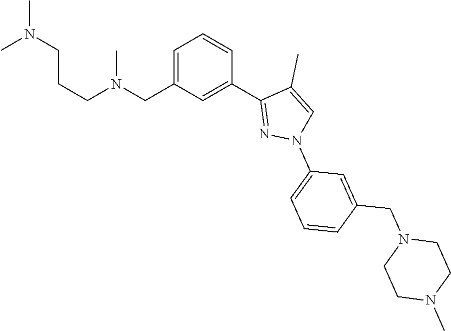 [SMILES: Cc1cn(-c2cccc(CN3CCN(C)CC3)c2)nc1-c1cccc(CN(C)CCCN(C)C)c1] 71 | N,N,N′-trimethyl-N′- [3-[4-methyl-1-[3-[(4- methylpiperazin-1-yl) methyl]phenyl]-1H- pyrazol-3-yl]phenyl] methyl]propane-1,3- diamine |
| 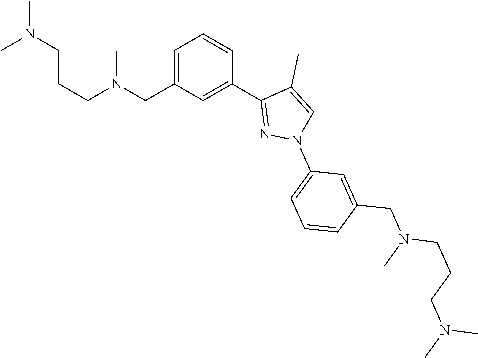 72 | N′-[3-[1-[3-[[3- (dimethylamino)propyl- methyl-amino]methyl] phenyl]-4-methyl-1H- pyrazol-3-yl]phenyl] methyl]-N,N,N′- trimethyl-propane- 1,3-diamine |

The compounds of the invention can be prepared in different ways with reactions known by the person skilled in the art. Reaction schemes as described in the example section illustrate by way of example different possible approaches.

The compounds of the invention are indeed able to repress the Aβ₁₋ₓ peptides production, in particular with low repression of Aβ_{x-38/40/42} released concentrations, more particularly with lower repression of Aβ_{x-38/40/42} concentrations compared to Aβ₁₋ₓ, and to modulate the ratio of autophagy markers, in particular LC3B II/I ratio and p62 expression.

Accordingly, in a particularly preferred embodiment, the invention relates to the use of compounds of Formula I and its sub-formulae, in particular those of Table 1 above, or pharmaceutically acceptable salts and solvates thereof, for repressing Aβ₁₋ₓ production, in particular with low repression of Aβ_{x-38/40/42} released concentrations, more particularly with lower repression of Aβ_{x-38/40/42} concentrations compared to Aβ₁₋ₓ, and modulating the ratio of autophagy markers, in particular LC3B II ratio and p62 expression.

### Applications

Unexpectedly, the inventors have discovered that the compounds of formula I according to the present invention may be used to rectify the metabolism of amyloid protein precursor (APP) by repressing the Aβ₁₋ₓ peptides production and modulating the ratio of autophagy markers.

In particular, and without wanting to be tied to any theory whatsoever, the inventors think that the activity of the compounds of the invention involves a mechanism relying on 1) the repression of Aβ₁₋ₓ production, in particular with low repression of Aβ_{x-38/40/42} released concentrations, more particularly with lower repression of Aβ_{x-38/40/42} concentrations compared to Aβ₁₋ₓ, and 2) a modulatory activity towards autophagy exemplified by modification of LC3B-II/LC3B-I ratio and p62 percentage of expression.

The compounds of the invention are therefore useful in the prevention and/or treatment of diseases involving formation of amyloid plaques and/or in which dysfunction of the APP metabolism occurs.

The invention thus also relates to a compound of the invention or a pharmaceutically acceptable salt or solvate thereof for use in treating and/or preventing a disease or disorder involving the formation of amyloid plaques and/or in which dysfunction of the APP metabolism occurs. Or in other terms, the invention also relates to a method of treating and/or preventing a disease or disorder involving the formation of amyloid plaques and/or in which dysfunction of the APP metabolism occurs, comprising the administration of a therapeutically effective amount of a compound or pharmaceutically acceptable salt or solvate of the invention, to a patient in need thereof. Preferably the patient is a warm-blooded animal, more preferably a human.

Diseases involving the formation of amyloid plaques and/or in which dysfunction of the APP metabolism occurs within the meaning of the present invention include, but are not limited to Alzheimer's disease (AD), Lewy body disease, Down syndrome, amyloid angiopathy, Parkinson's disease (PD), prion diseases, in particular Creutzfeldt-Jakob Disease (CJD), amyotrophic lateral sclerosis (ALS), and frontotemporal degeneration. In a particularly preferred embodiment, the disease involving the formation of amyloid plaques and/or in which dysfunction of the APP metabolism occurs is Alzheimer's disease.

Thus, in one embodiment, the invention relates to the compound of Formula I or a pharmaceutically acceptable salt or solvate thereof for use in treating and/or preventing a disease selected from the group consisting of Alzheimer's disease (AD), Lewy body disease, Down syndrome, amyloid angiopathy, Parkinson's disease (PD), Creutzfeldt-Jakob disease (CJD), amyotrophic lateral sclerosis (ALS) and frontotemporal degeneration. In particular, the invention relates to a compound of the invention or a pharmaceutically acceptable salt or solvate thereof for use in treating and/or preventing Alzheimer's disease (AD).

The invention further provides the use of a compound of the invention or a pharmaceutically acceptable salt or solvates thereof for the manufacture of a medicament for use in treating and/or preventing a disease or disorder involving the formation of amyloid plaques and/or in which dysfunction of the APP metabolism occurs. Preferably the patient is a warm-blooded animal, more preferably a human. The diseases or disorders involving the formation of amyloid plaques and/or in which dysfunction of the APP metabolism occurs are preferably those defined above.

According to a further feature of the present invention, there is provided a compound of the invention or a pharmaceutically acceptable salt or solvate for use in repressing Aβ₁₋ₓ production, in particular with low repression of Aβ_{x-38/40/42} released concentrations, more particularly with lower repression of Aβ_{x-38/40/42} concentrations compared to Aβ₁₋ₓ, in a patient in need of such treatment, comprising administering to said patient an effective amount of a compound of the present invention, or a pharmaceutically acceptable salt or solvate thereof. In other terms, the invention also provides a method for repressing Aβ₁₋ₓ production, in particular with low repression of Aβ_{x-38/40/42} released concentrations, more particularly with lower repression of Aβ_{x-38/40/42} concentrations compared to Aβ₁₋ₓ, in a patient in need of such treatment, which comprises administering to said patient an effective amount of a compound of the present invention, or a pharmaceutically acceptable salt or solvate thereof. Preferably, the patient is a warm-blooded animal, and even more preferably a human.

According to one embodiment, the compounds of the invention, and their pharmaceutically acceptable salts or solvates, may be administered as part of a combination therapy. Thus, are included within the scope of the present invention embodiments comprising co-administration of, and compositions and medicaments which contain, in addition to a compound of the present invention, a pharmaceutically acceptable salt or solvate thereof as an active ingredient, additional therapeutic agents and/or active ingredients. Such multiple-drug regimens, often referred to as combination therapy, may be used in the treatment and/or prevention of any disease or disorder involving the formation of amyloid plaques and/or in which dysfunction of the APP metabolism occurs, particularly those defined above.

Thus, the methods of treatment and pharmaceutical compositions of the present invention may employ the compounds of the invention or their pharmaceutically acceptable salts or solvates thereof in the form of monotherapy but said methods and compositions may also be used in the form of multiple therapies in which one or more compounds of Formula I or their pharmaceutically acceptable salts or solvates are co-administered in combination with one or more other therapeutic agents.

The invention also provides pharmaceutical compositions comprising a compound of the invention or a pharmaceutically acceptable salt or solvate thereof and at least one pharmaceutically acceptable carrier, diluent, excipient and/or adjuvant. As indicated above, the invention also covers pharmaceutical compositions which contain, in addition to a compound of the present invention, a pharmaceutically acceptable salt or solvate thereof as an active ingredient, additional therapeutic agents and/or active ingredients.

Another object of this invention is a medicament comprising at least one compound of the invention, or a pharmaceutically acceptable salt or solvate thereof, as an active ingredient.

Generally, for pharmaceutical use, the compounds of the invention may be formulated as a pharmaceutical preparation comprising at least one compound of the invention and at least one pharmaceutically acceptable carrier, diluent, excipient and/or adjuvant, and optionally one or more further pharmaceutically active compounds.

By means of non-limiting examples, such a formulation may be in a form suitable for oral administration, parenteral administration (such as by intravenous, intramuscular or subcutaneous injection or intravenous infusion), topical administration (including ocular), cerebral administration, sublingual administration, aerosol administration, for administration by inhalation, by a skin patch, by an implant, by a suppository, etc. Such suitable administration forms—which may be solid, semi-solid or liquid, depending on the manner of administration—as well as methods and carriers, diluents and excipients for use in the preparation thereof, will be clear to the skilled person; reference is made to the latest edition of Remington's Pharmaceutical Sciences.

### DEFINITIONS

The definitions and explanations below are for the terms as used throughout the entire application, including both the specification and the claims.

Unless otherwise stated any reference to compounds of the invention herein, means the compounds as such as well as their pharmaceutically acceptable salts and solvates.

When describing the compounds of the invention, the terms used are to be construed in accordance with the following definitions, unless indicated otherwise.

The term “halo” or “halogen” refers to the atoms of group 17 of the periodic table (halogens) and includes in particular fluorine, chlorine, bromine and iodine atoms.

The term “alkyl” by itself or as part of another substituent refers to a hydrocarbyl radical of Formula CₙH₂ₙ₊₁ wherein n is a number greater than or equal to 1.

The term “haloalkyl” alone or in combination, refers to an alkyl radical having the meaning as defined above wherein one or more hydrogens are replaced with a halogen as defined above. Non-limiting examples of such haloalkyl radicals include chloromethyl, 1-bromoethyl, fluoromethyl, difluoromethyl, trifluoromethyl, 1,1,1-trifluoroethyl and the like.

The term “cycloalkyl” as used herein is a monovalent, saturated, or unsaturated monocyclic or bicyclic hydrocarbyl group. Cycloalkyl groups may comprise 3 or more carbon atoms in the ring and generally, according to this invention comprise from 3 to 10, more preferably from 3 to 8 carbon atoms still more preferably from 3 to 6 carbon atoms. Examples of cycloalkyl groups include but are not limited to cyclopropyl, cyclobutyl, cyclopentyl, and cyclohexyl.

The term “heteroatom” as used herein refers to any atom that is not carbon or hydrogen. Non-limiting examples of such heteroatoms include nitrogen, oxygen, sulfur, and phosphorus. Preferred heteroatoms are nitrogen and oxygen.

The terms “heterocyclyl”, “heterocycloalkyl” or “heterocyclo” as used herein by itself or as part of another group refer to non-aromatic, fully saturated or partially unsaturated cyclic groups (for example, 3- to 7-member monocyclic, 7- to 11-member bicyclic, or containing a total of 3 to 10 ring atoms) which have at least one heteroatom in at least one carbon atom-containing ring. Each ring of the heterocyclic group containing a heteroatom may have 1, 2, 3 or 4 heteroatoms selected from nitrogen, oxygen and/or sulfur atoms, where the nitrogen and sulfur heteroatoms may optionally be oxidized, and the nitrogen heteroatoms may optionally be quaternized. The heterocyclic group may be attached at any heteroatom or carbon atom of the ring or ring system, where valence allows. Examples of heterocyclyl groups include, but are not limited to, aziridinyl, azetidinyl, pyrrolidinyl, piperidinyl, azepanyl, piperazinyl, morpholinyl and homopiperazinyl. Particular heterocyclyl group according to the invention are piperidinyl, piperazinyl, morpholinyl and homopiperazinyl.

More preferred heterocyclyl group according to the invention are piperidinyl, piperazinyl, and morpholinyl.

The term “aryl” as used herein refers to a polyunsaturated, aromatic hydrocarbyl group having a single ring (i.e. phenyl) or multiple aromatic rings fused together (e.g. naphthyl), typically containing 5 to 12 atoms; preferably 6 to 10, wherein at least one ring is aromatic. Examples of aryl groups include but are not limited to phenyl, naphthyl and anthracyl. Preferred aryl group according to the invention is phenyl.

The term “heteroaryl” as used herein by itself or as part of another group refers but is not limited to 5 to 12 carbon-atom aromatic rings or ring systems containing 1 to 2 rings which are fused together, typically containing 5 to 6 atoms; at least one of which is aromatic, in which one or more carbon atoms in one or more of these rings is replaced by oxygen, nitrogen and/or sulfur atoms where the nitrogen and sulfur heteroatoms may optionally be oxidized and the nitrogen heteroatoms may optionally be quaternized. Examples of heteroaryl groups include but are not limited to pyridyl, pyrazinyl, pyrimidinyl, pyridazinyl, triazinyl, quinolinyl, quinoxalinyl, quinazolinyl, furanyl, benzofuranyl, pyrrolyl, indolyl, thiophenyl, benzothiophenyl, imidazolyl, benzimidazolyl, pyrazolyl, indazolyl, oxazolyl, benzoxazolyl, isoxazolyl, benzisoxazolyl, thiazolyl and benzothiazolyl. Preferred heteroaryl group according to the invention is pyrrolyl.

The compounds of the invention containing a basic functional group may be in the form of pharmaceutically acceptable salts. Pharmaceutically acceptable salts of the compounds of the invention containing one or more basic functional groups include in particular the acid addition salts thereof. Suitable acid addition salts are formed from acids which form non-toxic salts. Examples include acetate, adipate, aspartate, benzoate, besylate, bicarbonate/carbonate, bisulfate/sulfate, borate, camsylate, cinnamate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Pharmaceutically acceptable salts of compounds of Formula I and sub-formulae may for example be prepared as follows:
(i) reacting the compound of Formula I or any of its sub-formulae with the desired acid; or
(ii) converting one salt of the compound of Formula I or any of its sub-formulae to another by reaction with an appropriate acid or by means of a suitable ion exchange column.

All these reactions are typically carried out in solution. The salt may precipitate from the solution and be collected by filtration or may be recovered by evaporation of the solvent.

The degree of ionization in the salt may vary from completely ionized to almost non-ionized.

The term “solvate” is used herein to describe a molecular complex comprising the compound of the invention and one or more pharmaceutically acceptable solvent molecules, for example, ethanol. The term “hydrate” is employed when said solvent is water.

The compounds of the invention include compounds of the invention as hereinbefore defined, including all polymorphs and crystal habits thereof, prodrugs and isomers thereof (including optical, geometric, and tautomeric isomers) and isotopically-labelled compounds of the invention.

In addition, although generally, with respect to the salts of the compounds of the invention, pharmaceutically acceptable salts are preferred, it should be noted that the invention in its broadest sense also includes non-pharmaceutically acceptable salts, which may for example be used in the isolation and/or purification of the compounds of the invention.

The term “patient” refers to a warm-blooded animal, more preferably a human, who/which is awaiting or receiving medical care or is or will be the object of a medical procedure.

The term “human” refers to subjects of both genders and at any stage of development (i.e. neonate, infant, juvenile, adolescent, adult). In one embodiment, the human is an adolescent or adult, preferably an adult.

The terms “treat”, “treating” and “treatment”, as used herein, are meant to include alleviating or abrogating a condition or disease and/or its attendant symptoms.

The terms “prevent”, “preventing” and “prevention”, as used herein, refer to a method of delaying or precluding the onset of a condition or disease and/or its attendant symptoms, barring a patient from acquiring a condition or disease, or reducing a patient's risk of acquiring a condition or disease.

The term “therapeutically effective amount” (or more simply an “effective amount”) as used herein means the amount of active agent or active ingredient which is sufficient to achieve the desired therapeutic or prophylactic effect in the individual to which it is administered.

The term “administration”, or a variant thereof (e.g., “administering”), means providing the active agent or active ingredient, alone or as part of a pharmaceutically acceptable composition, to the patient in whom/which the condition, symptom, or disease is to be treated or prevented.

By “pharmaceutically acceptable” is meant that the ingredients of a pharmaceutical composition are compatible with each other and not deleterious to the patient thereof.

The term “agonist” as used herein means a ligand that activates an intracellular response when it binds to a receptor.

The term “pharmaceutical vehicle” as used herein means a carrier or inert medium used as solvent or diluent in which the pharmaceutically active agent is formulated and/or administered. Non-limiting examples of pharmaceutical vehicles include creams, gels, lotions, solutions, and liposomes.

The present invention will be better understood by referring to the following examples and figures. These examples are intended to be representative of specific embodiments of the invention and are not intended as limiting the scope of the invention.

### FIGURES

FIG. 1: Effect of compounds 24, 57, 65, 67, 70, and 71 on the autophagic flux in SY5Y-APP^{wt} cells. SY5Y-APP^{wt} cells were treated for 24 h with compounds 24, 57, 65, 67, 70 and 71 (3 μM), CQ (20 μM), and Bafilomycin A1 (Baf A1) at 100 nM as control. Cell lysates were immunolabeled with the following antibodies: p62, LC3-B and β-actin.

### EXAMPLES

### Chemistry Examples

All commercial reagents and solvents were used without further purification. Organic layers obtained after extraction of aqueous solutions were dried over MgSO₄ and filtered before evaporation. Reaction yields were not optimized. Column chromatography was performed using Macherey-Nagel silica gel (230-400 mesh). ¹H and ¹³C NMR spectra were obtained using a Bruker DRX 300 spectrometer (operating at 300 MHz for ¹H and 75 MHz for ¹³C). Chemical shifts (S) were expressed in ppm relative to either TMS or residual proton signal in deuterated solvents. Mass spectra were recorded with an LC-MS (Waters Alliance Micromass ZQ 2000) using electrospray ionization. The purity of final compounds was verified by two types of high-pressure liquid chromatography (HPLC) columns: C18 Interchrom UPTISPHERE and C4 Interchrom UPTISPHERE. Analytical HPLC was performed on a Shimadzu LC-2010AHT system equipped with a UV detector set at 254 nm and 215 nm. The following eluent systems were used: buffer A (H₂O/TFA, 100:0.1) and buffer B (CH₃CN/H₂O/TFA, 80:20:0.1). Compounds were dissolved in 50 μL of buffer B and 950 μL of buffer A and injected into the system. HPLC retention times (HPLC t_{R}) were obtained at a flow rate of 0.2 mL/min using a gradient run from 100% of buffer A to 100% of buffer B over 30 min.

The following abbreviations are used:
ACN: Acetonitrile,
DCE: 1,2-dichloroethane,
DCM: Dichloromethane,
DMF: N,N-dimethylformamide,
DMSO: Dimethylsulfoxide,
eq: Equivalent,
ESI: Electrospray ionization,
EtOH: Ethanol,
HPLC: High-performance liquid chromatography,
MeOH: Methanol,
Mp: Melting point,
MS: Mass spectrometry,
NMR: Nuclear magnetic resonance,
rt: Room temperature,
THF: tetrahydrofuran,
TLC: Thin layer chromatography,
t_{R}: Retention time.

### General Procedure A

Dimethylformamide (81.5 mmol) was cooled to 0°C with a salt/ice bath. Phosphorus oxychloride (22.5 mmol) was added dropwise with the temperature maintained below 0°C. The mixture was then stirred for 40 min at 0°C. Hydrazone (5.39 mmol) was added and the reaction mixture was allowed to warm to room temperature. After 2 h, the temperature was increased to 50°C. The reaction was stirred at this temperature for 4 h. The mixture was then added to crushed ice and stirred for 1 h. Potassium carbonate was added until pH=8 and the mixture was extracted twice with methylene chloride. The combined organic layers were washed with brine, dried, and evaporated. The residue was purified by column chromatography (DCM/MeOH/NH₄OH=9:1:0.1).

### General Procedure B

To a solution of alkylamine (3.73 mmol), 37% formaldehyde in water (22.4 mmol) and acetic acid (22.4 mmol) in methanol (20 mL) was slowly added over 30 min sodium triacetoxyborohydride (18.7 mmol). The mixture was stirred until the completion of the reaction. A solution an aqueous carbonate potassium (10%) and ethyl acetate were added to the residue and the mixture was stirred for 10 min. The layers were separated, and the aqueous layer was extracted twice with ethyl acetate. The combined organic layers were washed with brine, dried and evaporated. The residue was purified by column chromatography (DCM/MeOH—NH₃ sat=95:5).

### General Procedure C

LiAlH₄ (1 M in THF, 2.82 mmol) was added to 20 mL of anhydrous THF under nitrogen. The solution was cooled to 0°C with an ice bath and a solution of ester (1.88 mmol) in 20 mL of anhydrous THF was added dropwise. The reaction was stirred at 0°C for 20 min and rt for 1 h. The mixture was then cooled with an ice bath and 0.11 mL of H₂O was added, followed by 0.11 mL of 15% NaOH and 0.33 mL of H₂O. The solid was isolated by filtration and washed with THF. The filtrate was evaporated. The residue was purified by column chromatography (DCM/MeOH/NH₄OH=9:1:0.1).

### General Procedure D

To a solution of the desired compound (1.42 mmol) in chloroform (25 mL) was added manganese (IV) oxide (14.2 mmol). This was stirred at rt for 24 h. More manganese (IV) oxide (14.2 mmol) was added, and the mixture was stirred for 24 h. The solid was filtered off and washed with methylene chloride. The filtrate was washed with 10% K₂CO₃ and with brine. The organic layer was dried and evaporated to give the aldehyde which was used in the next step without further purification.

### General Procedure E

To a solution of benzaldehyde (0.265 mmol), amine (0.45 mmol), and acetic acid (0.53 mmol) in DCE (4 mL) was added sodium triacetoxyborohydride (0.53 mmol). The reaction mixture was stirred under nitrogen for 24 h. 10% K₂CO₃ was added, and the layers were separated. The aqueous layer was extracted twice with methylene chloride. The combined organic layers were washed with brine, dried, and evaporated. The residue was purified by column chromatography (DCM/MeOH—NH₃ sat=9:1).

### Methyl 4-hydrazinobenzoate hydrochloride (3)

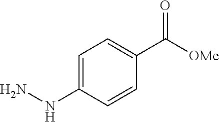 [SMILES: COC(=O)c1ccc(NN)cc1]

A solution of methyl 4-aminobenzoate (1) (6 g, 39.7 mmol) in HCl 37% (40 mL) was brought to −5°C with a salt/ice bath. A solution of sodium nitrite (3 g, 43.5 mmol) in water (22 mL) was slowly added over 1 h while maintaining the temperature below 0°C. The solution was then stirred at 0°C for 40 min and a solution of tin(II) chloride (13.86 g, 48.7 mmol) in HCl 37% (20 mL) was added dropwise while maintaining at 0°C. The mixture was stirred for another 20 min at 0°C and 2 h 30 at room temperature. The precipitate was collected by filtration, washed with 40 mL of ice-cold water, and dried to give 8.6 g of a white solid which was used for the next step without further purification.

### Methyl 3-hydrazinobenzoate hydrochloride (4)

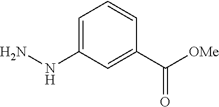 [SMILES: COC(=O)c1cccc(NN)c1]

A solution of methyl 3-aminobenzoate (2) (4 g, 26.5 mmol) in HCl 37% (40 mL) was brought to −5°C with a salt/ice bath. A solution of sodium nitrite (2 g, 29 mmol) in water (15 mL) was slowly added over 1 h while maintaining the temperature below 3°C. The solution was then stirred at 0°C for 30 min and a solution of tin(II) chloride (9.24 g, 48.7 mmol) in 37% HCl (20 mL) was added dropwise while maintaining the temperature at 0°C. The mixture was stirred for another 30 min at 0°C and 2 h at room temperature. The precipitate was collected by filtration, washed subsequently with 15 mL of ice-cold water and with ether, and dried to give 6.55 g of a white solid which was used for the next step without further purification.

### 1-[4-[3-(dimethylamino)propyl]phenyl]ethanone (5)

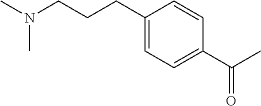

In a sealed tube, a mixture of 1-[4-(3-bromopropyl)phenyl]ethanone (5.6 g, 23.2 mmol) and dimethylamine (2 M in methanol, 34.8 mL, 69.6 mmol) were heated at 65°C for 15 h. The solvent was evaporated and 10% K₂CO₃ (100 mL) was added. The mixture was extracted twice with ethyl acetate and the combined organic layers were washed with brine, dried, and evaporated. The residue was purified by column chromatography (DCM/MeOH=9:1) to give 3.66 g (59%) of the product as a yellow oil.

¹H NMR (CDCl₃, 300 Mz): δ 7.89 (d, J=8.4 Hz, 2H), 7.29 (d, J=8.4 Hz, 2H), 2.70 (t, J=7.6 Hz, 2H), 2.58 (s, 3H), 2.29 (t, J=7.1 Hz, 2H), 2.23 (s, 6H), 1.81 (quint, J=7.5 Hz, 2H).

¹³C NMR (CDCl₃, 75 MHz): δ 197.9, 148.2, 135.1, 128.7, 128.6, 59.0, 45.5, 33.7, 29.1, 26.6.

MS (ESI) m/z 206 [M+H]⁺.

### Methyl 4-[2-[1-[4-[3-(dimethylamino)propyl]phenyl]ethylidene]hydrazino]benzoate (6)

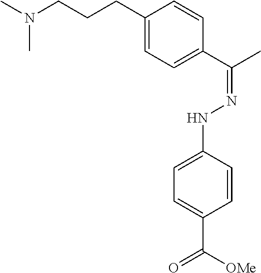 [SMILES: COC(=O)c1ccc(N/N=C(/C)c2ccc(CCCN(C)C)cc2)cc1]

To a solution of 3 (8.5 g) in methanol (220 mL) was added 5 (3.45 g, 16.81 mmol). The mixture was stirred at rt for 48 h. The precipitate was collected by filtration and washed with methanol to give 4.08 g (62%) of the product as a yellowish solid.

¹H NMR (CD₃SOCD₃, 300 Mz): δ 10.88 (br, 1H), 9.89 (s, 1H), 7.83 (d, J=8.8 Hz, 1H), 7.75 (d, J=8.3 Hz, 1H), 7.33 (d, J=8.8 Hz, 1H), 7.27 (d, J=8.3 Hz, 1H), 3.78 (s, 3H), 3.02-2.99 (m, 2H), 2.71-2.63 (m, 8H), 2.30 (s, 3H), 2.05-1.95 (m, 2H).

¹³C NMR (CD₃SOCD₃, 75 MHz): δ 166.2, 150.0, 143.5, 140.5, 136.8, 130.8, 128.3, 125.6, 119.1, 112.0, 56.0, 51.4, 41.9, 31.6, 25.1, 13.3. MS (ESI) m/z 354 [M+H]⁺.

### Methyl 3-[2-[1-[4-[3-(dimethylamino)propyl]phenyl]ethylidene]hydrazino]benzoate hydrochloride (7)

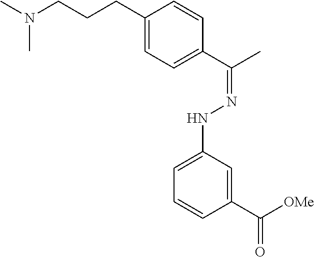 [SMILES: COC(=O)c1cccc(N/N=C(/C)c2ccc(CCCN(C)C)cc2)c1]

To a solution of 4 (10.9 g) in methanol (105 mL) was added 5 (3.45 g, 16.81 mmol). The mixture was stirred at rt for 48 h. The precipitate was collected by filtration and washed with methanol to give 6.19 g (94%) of the product as a light brown solid.

¹H NMR (CD₃SOCD₃, 300 Mz): δ 10.30 (br, 1H), 9.52 (s, 1H), 7.84-7.83 (m, 1H), 7.73 (d, J=8.2 Hz, 2H), 7.53-7.49 (m, 1H), 7.36-7.34 (m, 2H), 7.26 (d, J=8.3 Hz, 2H), 3.85 (s, 3H), 3.05-3.00 (m, 2H), 2.73 (s, 6H), 2.65 (t, J=7.7 Hz, 2H), 2.26 (s, 3H), 2.03-1.93 (m, 2H).

¹³C NMR (CD₃SOCD₃, 75 MHz): δ 6166.6, 146.4, 141.9, 140.1, 137.1, 130.3, 129.3, 128.3, 125.4, 119.3, 117.1, 113.3, 56.1, 52.0, 42.0, 31.6, 25.2, 13.1. MS (ESI) m/z 354 [M+H]⁺.

### Methyl 4-[3-[4-[3-(dimethylamino)propyl]phenyl]-4-formyl-1H-pyrazol-1-yl]benzoate (8)

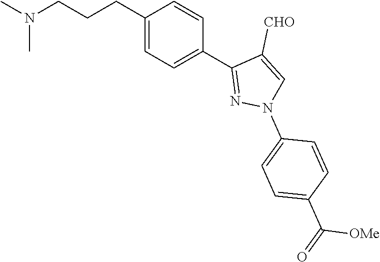 [SMILES: COC(=O)c1ccc(-n2cc(C=O)c(-c3ccc(CCCN(C)C)cc3)n2)cc1]

Compound 8 was synthesized according to General procedure A from compound 6. 87% yield (white solid).

¹H NMR (CDCl₃, 300 Mz): δ 10.03 (s, 1H), 8.59 (s, 1H), 8.14 (d, J=8.8 Hz, 2H), 7.86 (d, J=8.8 Hz, 2H), 7.72 (d, J=8.1 Hz, 2H), 7.31 (d, J=8.1 Hz, 2H), 3.92 (s, 3H), 2.70 (t, J=7.5 Hz, 2H), 2.32 (t, J=7.1 Hz, 2H), 2.23 (s, 6H), 1.82 (quint, J=7.4 Hz, 2H).

¹³C NMR (CDCl₃, 75 MHz): δ 185.1, 166.0, 155.2, 144.0, 142.1, 131.3, 131.2, 129.3, 128.9, 128.6, 123.0, 119.0, 59.1, 52.4, 45.5, 33.5, 29.3. MS (ESI) m/z 392 [M+H]⁺.

### Methyl 3-[3-[4-[3-(dimethylamino)propyl]phenyl]-4-formyl-pyrazol-1-yl]benzoate (9)

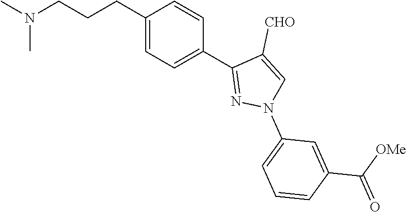 [SMILES: COC(=O)c1cccc(-n2cc(C=O)c(-c3ccc(CCCN(C)C)cc3)n2)c1]

Compound 9 was synthesized according to General procedure A from compound 7. 67% yield (white solid).

¹H NMR (CDCl₃, 300 Mz): δ 10.06 (s, 1H), 8.61 (s, 1H), 8.44-8.42 (m, 1H), 8.05-8.02 (m, 1H), 7.76 (d, J=8.1 Hz, 2H), 7.58 (t, J=7.9 Hz, 1H), 7.33 (d, J=8.1 Hz, 2H), 3.96 (s, 3H), 2.72 (t, J=7.5 Hz, 2H), 2.33 (t, J=7.1 Hz, 2H), 2.23 (s, 6H), 1.85 (quint, J=7.3 Hz, 2H).

¹³C NMR (CDCl₃, 75 MHz): δ 185.1, 165.9, 154.9, 143.9, 139.2, 131.8, 131.2, 129.9, 128.9, 128.9, 128.7, 128.7, 123.8, 122.8, 120.3, 59.2, 52.5, 45.5, 33.5, 29.3. MS (ESI) m/z 392 [M+H]⁺.

### Methyl 4-[4-(dimethylaminomethyl)-3-[4-[3-(dimethylamino)propyl]phenyl]pyrazol-1-yl]benzoate (10)

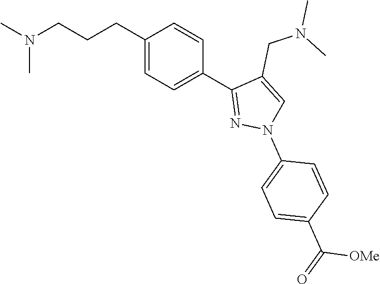 [SMILES: COC(=O)c1ccc(-n2cc(CN(C)C)c(-c3ccc(CCCN(C)C)cc3)n2)cc1]

A mixture of 8 (0.9 g, 2.3 mmol), dimethylamine (2 M in THF, 2.3 mL, 4.6 mmol), sodium triacetoxyborohydride (0.88 g, 4.14 mmol), and acetic acid (0.24 mL, 4.14 mmol) in DCE (10 mL) was stirred at rt under nitrogen for 4 h. 10% K₂CO₃ and methylene chloride were added. The layers were separated, and the aqueous layer was extracted twice with methylene chloride. The combined organic layers were washed with brine, dried, and evaporated. The residue was purified by column chromatography (DCM/MeOH/NH₄OH=9:1:0.05) to give 0.82 g (84%) of the product as a white solid.

¹H NMR (CD₃OD, 300 Mz): 88.22 (s, 1H), 8.03 (d, J=8.8 Hz, 2H), 7.84 (d, J=8.8 Hz, 2H), 7.72 (d, J=8.1 Hz, 2H), 7.26 (d, J=8.1 Hz, 2H), 3.86 (s, 3H), 3.43 (s, 2H), 2.64 (t, J=7.6 Hz, 2H), 2.37-2.32 (m, 2H), 2.22 (s, 6H), 2.20 (s, 6H), 1.86-1.79 (m, 2H).

¹³C NMR (CD₃OD, 75 MHz): δ 167.6, 154.5, 144.4, 143.3, 132.0, 131.9, 130.3, 129.5 (2C), 128.5, 119.8, 118.9, 60.1, 54.1, 52.6, 45.4, 45.2, 34.3, 29.9. MS (ESI) m/z 421 [M+H]⁺.

### Methyl 3-[4-(dimethylaminomethyl)-3-[4-[3-(dimethylamino)propyl]phenyl]pyrazol-1-yl]benzoate (11)

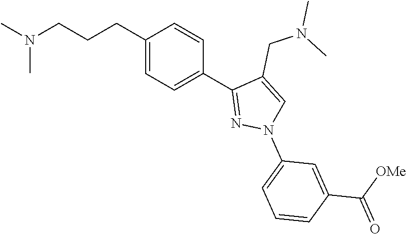 [SMILES: COC(=O)c1cccc(-n2cc(CN(C)C)c(-c3ccc(CCCN(C)C)cc3)n2)c1]

To a solution of 9 (0.7 g, 1.79 mmol), dimethylamine (2 M in THF, 1.79 mL, 3.58 mmol) and acetic acid (0.18 mL, 3.22 mmol) in DCE (8 mL) was added sodium triacetoxyborohydride (0.68 g, 3.22 mmol). The reaction mixture was stirred at rt for 4 h. 10% K₂CO₃ was added and the layers were separated. The organic layer was extracted twice with methylene chloride. The combined organic layers were washed with brine, dried, and evaporated. The residue was purified by column chromatography (DCM/MeOH=95:5 to 8:2) to give 0.64 g (85%) of the product as a colorless oil.

¹H NMR (CD₃OD, 300 Mz): δ 8.37 (s, 1H), 8.19 (s, 1H), 7.97-7.94 (m, 1H), 7.84 (d, J=7.9 Hz, 1H), 7.71 (d, J=8.1 Hz, 2H), 7.49 (t, J=7.9 Hz, 1H), 7.25 (d, J=8.1 Hz, 2H), 3.88 (s, 3H), 3.44 (s, 2H), 2.62 (t, J=7.6 Hz, 2H), 2.36 (t, J=7.4 Hz, 2H), 2.24 (s, 6H), 2.20 (s, 6H), 1.81 (quint, J=7.8 Hz, 2H).

¹³C NMR (CD₃OD, 75 MHz): δ 167.4, 154.0, 143.0, 141.3, 132.6, 132.0, 130.8, 130.1, 129.5 (2C), 127.9, 123.7, 120.2, 119.2, 59.9, 54.0, 52.8, 45.3, 45.2, 34.2, 29.7. MS (ESI) m/z 421 [M+H]⁺.

### Methyl 4-[4-(3-aminopropyl)-3-[4-[3-(dimethylamino)propyl]phenyl]pyrazol-1-yl]benzoate (12)

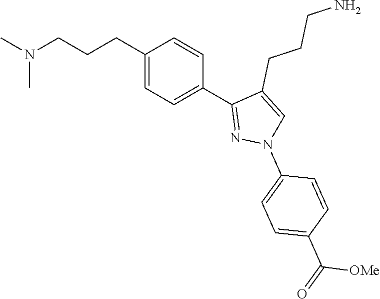 [SMILES: COC(=O)c1ccc(-n2cc(CCCN)c(-c3ccc(CCCN(C)C)cc3)n2)cc1]

To a suspension of NaH (60% dispersion in mineral oil, 0.17 g, 4.32 mmol) in anhydrous THF (15 mL) at 0°C under nitrogen was added dropwise diethyl cyanomethylphosphonate (0.65 mL, 3.99 mmol). The mixture was stirred at 0°C for 30 min and a solution of 8 (1.3 g, 3.32 mmol) in anhydrous THF (20 mL) was added dropwise. The reaction mixture was stirred at 0°C for 10 min and then was allowed to warm to rt. After 2 h, the solvent was evaporated, and the residue was purified by column chromatography (DCM/MeOH/NH₄OH=9:1:0.02) to give 1.175 g (85%) of a white solid. MS (ESI) m/z 415 [M+H]⁺.

A mixture of the intermediate (1.16 g, 2.80 mmol), Raney Nickel (0.12 g), and 10% Pd/C (0.12 g) in methanol saturated with ammonia (110 mL) and THF (10 mL) was stirred under a hydrogen atmosphere for 30 h. The catalyst was filtered off and the filtrate was evaporated. The residue was purified by column chromatography (DCM/MeOH/NH₄OH=9:1:0.1) to give 0.795 g (68%) of the product as a colorless oil. ¹H NMR (CDCl₃, 300 Mz): δ 8.12 (d, J=8.9 Hz, 2H), 7.88 (s, 1H), 7.82 (d, J=8.9 Hz, 2H), 7.65 (d, J=8.1 Hz, 2H), 7.28 (d, J=8.1 Hz, 2H), 3.93 (s, 3H), 2.81-2.67 (m, 6H), 2.37-2.32 (m, 2H), 2.26 (s, 6H), 2.00 (br, 2H), 1.90-1.76 (m, 4H). ¹³C NMR (CDCl₃, 75 MHz): δ 166.6, 152.6, 143.4, 142.3, 131.2, 131.0, 128.8, 128.0, 127.3, 126.2, 122.2, 117.7, 59.3, 52.3, 45.5, 41.8, 33.8, 33.5, 29.3, 22.1. MS (ESI) m/z 421 [M+H]⁺.

### Methyl 3-[4-(3-aminopropyl)-3-[4-[3-(dimethylamino)propyl]phenyl]pyrazol-1-yl]benzoate (13)

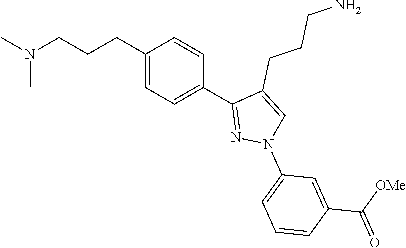 [SMILES: COC(=O)c1cccc(-n2cc(CCCN)c(-c3ccc(CCCN(C)C)cc3)n2)c1]

To a suspension of NaH (60% dispersion in mineral oil, 0.1 g, 2.5 mmol) in 12 mL of anhydrous THF at 0°C under nitrogen was added dropwise a solution of diethyl cyanomethylphosphonate (0.38 mL, 2.32 mmol) in anhydrous THF (3 mL). The mixture was stirred at 0°C for 30 min and 9 (0.7 g, 1.79 mmol) was slowly added. The reaction mixture was allowed to warm to room temperature. After 2 h, water and ethyl acetate were added and the layers were separated. The aqueous layer was extracted with ethyl acetate. The combined organic layers were washed with brine, dried, and evaporated. The residue was purified by column chromatography (DCM/MeOH—NH₃ sat=9:1) to give 527 mg (71%) of a colorless oil. MS (ESI) m/z 415 [M+H]⁺.

A mixture of the intermediate (0.5 g, 1.21 mmol), Raney Nickel (50 mg) and 10% Pd/C (50 mg) in methanol saturated with ammonia (60 mL) was stirred under a hydrogen atmosphere for 30 h. The catalyst was filtered off and the filtrate was evaporated. The residue was purified by column chromatography (DCM/MeOH/NH₄OH=9:1:0.1) to give 317 mg (62%) of the product as a colorless oil.

¹H NMR (CDCl₃, 300 Mz): δ 8.28-8.27 (m, 1H), 7.96-7.92 (m, 1H), 7.86-7.82 (m, 1H), 7.81 (s, 1H), 7.60 (d, J=8.1 Hz, 2H), 7.43 (t, J=7.9 Hz, 1H), 7.22 (d, J=8.1 Hz, 2H), 3.88 (s, 3H), 2.73-2.60 (m, 6H), 2.26 (t, J=7.1 Hz, 2H), 2.18 (s, 6H), 1.90 (br, 2H), 1.82-1.68 (m, 4H).

¹³C NMR (CDCl₃, 75 MHz): δ 166.4, 151.8, 142.0, 140.1, 131.3, 131.0, 129.5, 128.5, 127.7, 126.6, 125.9, 122.7, 121.5, 118.9, 59.1, 52.2, 45.4, 41.6, 33.9, 33.3, 29.2, 22.0. MS (ESI) m/z 421 [M+H]⁺.

### Methyl 4-[4-[3-(dimethylamino)propyl]-3-[4-[3-(dimethylamino) propyl]phenyl]pyrazol-1-yl]benzoate (14)

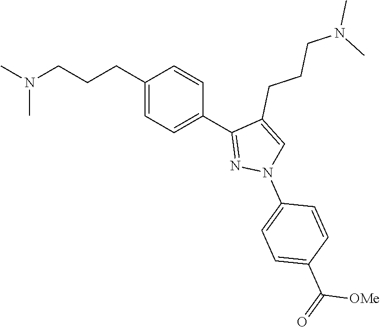

Compound 14 was obtained according to General procedure B from compound 8. 90% yield (white solid). ¹H NMR (CDCl₃, 300 Mz): δ 8.11 (d, J=8.8 Hz, 2H), 7.88 (s, 1H), 7.82 (d, J=8.8 Hz, 2H), 7.65 (d, J=8.1 Hz, 2H), 7.27 (d, J=8.1 Hz, 2H), 3.92 (s, 3H), 2.74-2.66 (m, 4H), 2.37-2.32 (m, 4H), 2.26 (s, 6H), 2.23 (s, 6H), 1.89-1.77 (m, 4H). ¹³C NMR (CDCl₃, 75 MHz): δ 166.6, 152.6, 143.4, 142.2, 131.2, 131.0, 128.7, 128.0, 127.2, 126.2, 122.3, 117.7, 59.3, 59.2, 52.3, 45.5, 45.5, 33.5, 29.3, 28.2, 22.6. MS (ESI) m/z 449 [M+H]⁺.

### Methyl 3-[4-[3-(dimethylamino)propyl]-3-[4-[3-(dimethylamino)propyl]phenyl]pyrazol-1-yl]benzoate (15)

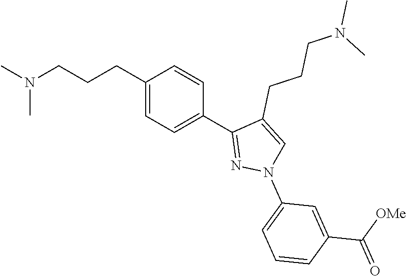 [SMILES: COC(=O)c1cccc(-n2cc(CCCN(C)C)c(-c3ccc(CCCN(C)C)cc3)n2)c1]

Compound 15 was obtained according to General procedure B from compound 9. 82% yield (colorless oil). ¹H NMR (CD₃OD, 300 Mz): δ 8.37-8.36 (m, 1H), 8.10 (s, 1H), 7.98-7.94 (m, 1H), 7.85-7.83 (m, 1H), 7.61 (d, J=8.1 Hz, 2H), 7.50 (t, J=8.0 Hz, 1H), 7.26 (d, J=8.1 Hz, 2H), 3.89 (s, 3H), 2.66-2.61 (m, 4H), 2.39-2.31 (m, 4H), 2.24 (s, 6H), 2.20 (s, 6H), 1.87-1.74 (m, 4H). ¹³C NMR (CD₃OD, 75 MHz): δ 167.6, 153.0, 143.0, 141.5, 132.6, 132.5, 130.8, 129.6, 129.0, 128.2, 127.7, 123.6, 122.7, 120.1, 60.1, 60.0, 52.9, 45.3 (2C), 34.3, 29.9, 28.6, 23.5. MS (ESI) m/z 449 [M+H]⁺.

### Example 1: [4-[4-(dimethylaminomethyl)-3-[4-[3-(dimethylamino)propyl]phenyl]-1H-pyrazol-1-yl]phenyl]methanol (16)

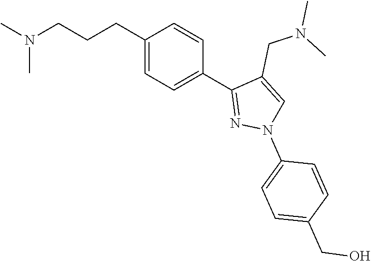 [SMILES: CN(C)CCCc1ccc(-c2nn(-c3ccc(CO)cc3)cc2CN(C)C)cc1]

Compound 16 was obtained according to General procedure C from compound 10. 88% yield (colorless oil).

¹H NMR (CD₃OD, 300 Mz): 88.18 (s, 1H), 7.76 (d, J=8.6 Hz, 2H), 7.68 (d, J=8.2 Hz, 2H), 7.46 (d, J=8.6 Hz, 2H), 7.29 (d, J=8.1 Hz, 2H), 4.63 (s, 2H), 3.50 (s, 2H), 2.66 (t, J=7.6 Hz, 2H), 2.39-2.34 (m, 2H), 2.24 (s, 6H), 2.22 (s, 6H), 1.89-1.78 (m, 2H).

¹³C NMR (CD₃OD, 75 MHz): δ 153.9, 143.2, 141.3, 140.3, 132.1, 130.4, 129.6, 129.6, 129.1, 119.9, 118.6, 64.5, 60.1, 53.9, 45.4, 45.1, 34.3, 30.0. MS (ESI) m/z 393 [M+H]⁺.

### Example 2: [4-[4-[3-(dimethylamino)propyl]-3-[4-[3-(dimethylamino)propyl]phenyl]-1H-pyrazol-1-yl]phenyl]methanol (17)

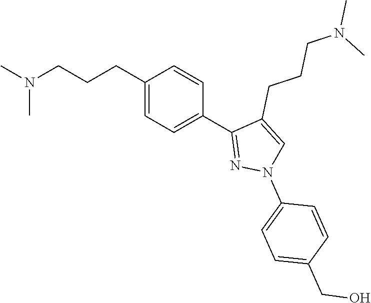

Compound 17 was obtained according to General procedure C from compound 14. 89% yield (white solid).

¹H NMR (CD₃OD, 300 Mz): 88.08 (s, 1H), 7.74 (d, J=8.6 Hz, 2H), 7.61 (d, J=8.2 Hz, 2H), 7.44 (d, J=8.7 Hz, 2H), 7.29 (d, J=8.2 Hz, 2H), 4.62 (s, 2H), 2.69-2.64 (m, 4H), 2.38-2.30 (m, 4H), 2.23 (s, 6H), 2.19 (s, 6H), 1.88-1.72 (m, 4H).

¹³C NMR (CD₃OD, 75 MHz): δ 152.7, 143.0, 141.0, 140.4, 132.6, 129.6, 129.1, 129.1, 128.4, 122.2, 119.7, 64.6, 60.2, 60.1, 45.4 (2C), 34.3, 30.0, 28.8, 23.4. MS (ESI) m/z 421 [M+H]⁺.

### [3-[4-(Dimethylaminomethyl)-3-[4-[3-(dimethylamino)propyl]phenyl]-1H-pyrazol-1-yl]phenyl]methanol (18)

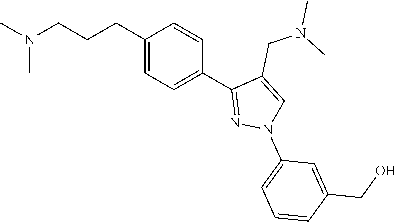 [SMILES: CN(C)CCCc1ccc(-c2nn(-c3cccc(CO)c3)cc2CN(C)C)cc1]

Compound 18 was obtained according to General procedure C from compound 11. 92% yield (colorless oil).

¹H NMR (CDCl₃, 300 Mz): δ 7.94 (s, 1H), 7.76-7.73 (m, 3H), 7.59 (d, J=8.1 Hz, 1H), 7.31 (t, J=7.7 Hz, 1H), 7.23-7.16 (m, 3H), 5.89 (s, 1H), 4.63 (s, 2H), 3.42 (s, 2H), 2.61 (t, J=7.5 Hz, 2H), 2.28 (t, J=7.0 Hz, 2H), 2.22 (s, 6H), 2.18 (s, 6H), 1.79 (quint, J=7.3 Hz, 2H).

¹³C NMR (CDCl₃, 75 MHz): δ 151.8, 143.5, 141.3, 139.7, 130.7, 129.0, 128.2, 128.0, 127.8, 123.9, 117.8, 116.9, 116.4, 63.6, 58.7, 53.4, 44.9, 44.8, 33.1, 28.6. MS (ESI) m/z 393 [M+H]⁺.

### [3-[4-[3-(dimethylamino)propyl]-3-[4-[3-(dimethylamino)propyl]phenyl]-1H-pyrazol-1-yl]phenyl]methanol (19)

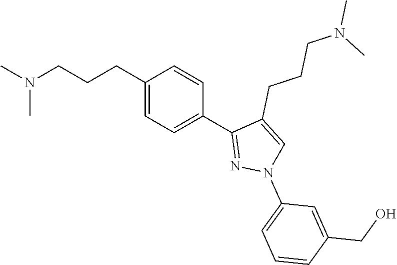 [SMILES: CN(C)CCCc1ccc(-c2nn(-c3cccc(CO)c3)cc2CCCN(C)C)cc1]

Compound 19 was obtained according to General procedure C from compound 15. 88% yield (colorless oil).

¹H NMR (CD₃OD, 300 Mz): δ 8.08 (s, 1H), 7.79 (s, 1H), 7.67-7.60 (m, 3H), 7.42 (t, J=7.7 Hz, 1H), 7.29-7.27 (m, 3H), 4.67 (s, 2H), 2.69-2.63 (m, 4H), 2.38-2.30 (m, 4H), 2.23 (s, 6H), 2.19 (s, 6H), 1.88-1.72 (m, 4H).

¹³C NMR (CD₃OD, 75 MHz): δ 152.8, 144.7, 143.1, 141.4, 132.6, 130.5, 129.6, 129.1, 128.5, 125.6, 122.3, 118.6, 118.1, 64.8, 60.2, 60.1, 45.4 (2C), 34.3, 30.0, 28.8, 23.4. MS (ESI) m/z 421 [M+H]⁺.

### 4-[4-(dimethylaminomethyl)-3-[4-[3-(dimethylamino)propyl]phenyl]pyrazol-1-yl]benzaldehyde (20)

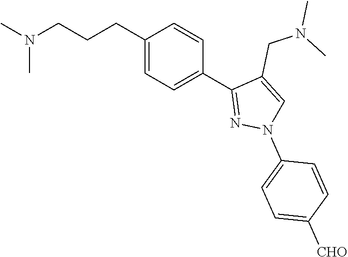 [SMILES: CN(C)CCCc1ccc(-c2nn(-c3ccc(C=O)cc3)cc2CN(C)C)cc1]

Compound 20 was obtained according to General procedure D from compound 16. 72% yield (colorless oil).

¹H NMR (CDCl₃, 300 Mz): δ 9.96 (s, 1H), 8.01 (s, 1H), 7.95-7.89 (m, 4H), 7.78 (d, J=8.2 Hz, 2H), 7.26 (d, J=8.2 Hz, 2H), 3.44 (s, 2H), 2.67 (t, J=7.6 Hz, 2H), 2.32-2.27 (m, 8H), 2.21 (s, 6H), 1.86-1.76 (m, 2H).

¹³C NMR (CDCl₃, 75 MHz): δ 191.0, 153.5, 144.2, 142.5, 133.7, 131.3, 130.4, 128.6, 128.3, 128.0, 120.3, 118.2, 59.2, 53.9, 45.5, 45.3, 33.5, 29.3. MS (ESI) m/z 391 [M+H]⁺.

### 4-[4-[3-(dimethylamino)propyl]-3-[4-[3-(dimethylamino)propyl]phenyl]-1H-pyrazol-1-yl]benzaldehyde (21)

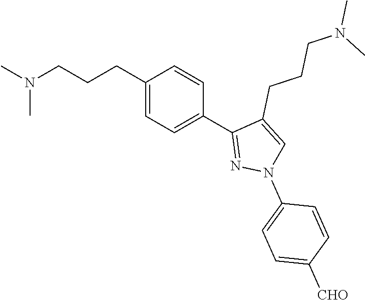

Compound 21 was obtained according to General procedure D from compound 17. 76% yield (yellow oil).

¹H NMR (50°C, CD₃OD, 300 Mz): δ 9.92 (s, 1H), 8.16 (s, 1H), 7.95 (s, 4H), 7.64-7.61 (m, 2H), 7.29-7.27 (m, 2H), 2.70-2.64 (m, 4H), 2.39-2.31 (m, 4H), 2.24 (s, 6H), 2.20 (s, 6H), 1.86-1.75 (m, 4H).

¹³C NMR (50°C, CD₃OD, 75 MHz): δ 192.7, 154.2, 145.6, 143.4, 135.3, 132.3, 132.2, 129.6, 129.1, 128.5, 123.7, 119.3, 60.2, 60.2, 45.4 (2C), 34.4, 29.8, 28.7, 23.5. MS (ESI) m/z 419 [M+H]⁺.

### 3-[4-(Dimethylaminomethyl)-3-[4-[3-(dimethylamino)propyl]phenyl]-1H-pyrazol-1-yl]benzaldehyde (22)

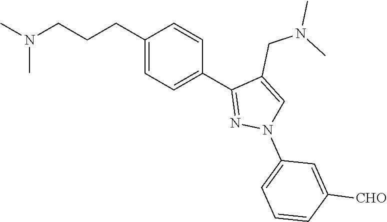 [SMILES: CN(C)CCCc1ccc(-c2nn(-c3cccc(C=O)c3)cc2CN(C)C)cc1]

Compound 22 was obtained according to General procedure D from compound 18. 60% yield (yellow oil).

¹H NMR (CDCl₃, 300 Mz): δ 10.04 (s, 1H), 8.24-8.23 (m, 1H), 8.08-8.04 (m, 1H), 8.01 (s, 1H), 7.82 (d, J=8.1 Hz, 2H), 7.72 (d, J=7.6 Hz, 1H), 7.58 (t, J=7.8 Hz, 1H), 7.29 (d, J=8.2 Hz, 2H), 3.45 (s, 2H), 2.69 (t, J=7.6 Hz, 2H), 2.35-2.29 (m, 8H), 2.24 (s, 6H), 1.84 (quint, J=7.4 Hz, 2H).

¹³C NMR (CD₃OD, 75 MHz): δ 193.2, 154.3, 143.3, 141.9, 139.1, 132.0, 130.3, 129.6, 129.5, 128.2, 125.0, 119.9, 119.4, 118.7, 60.1, 54.0, 45.4, 45.2, 34.3, 29.9. MS (ESI) m/z 391 [M+H]⁺.

### 3-[4-[3-(dimethylamino)propyl]-3-[4-[3-(dimethylamino)propyl]phenyl]pyrazol-1-yl]benzaldehyde (23)

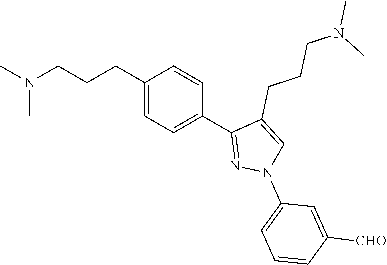

Compound 23 was obtained according to General procedure D from compound 19. 66% yield (colorless oil).

¹H NMR (CD₃OD, 50°C, 300 Mz): δ 10.03 (s, 1H), 8.28-8.27 (m, 1H), 8.16 (s, 1H), 8.09-8.04 (m, 1H), 7.79-7.76 (m, 1H), 7.66-7.59 (m, 3H), 7.31-7.27 (m, 2H), 2.73-2.66 (m, 4H), 2.49-2.40 (m, 4H), 2.31 (s, 6H), 2.25 (s, 6H), 1.89-1.78 (m, 4H).

¹³C NMR (CD₃OD, 50°C, 75 MHz): δ 193.3, 153.5, 143.1, 142.1, 139.3, 132.5, 131.4, 129.6, 129.2, 128.4, 128.0, 125.1, 122.9, 119.8, 60.0, 60.0, 45.2, 45.2, 34.2, 29.5, 28.5, 23.3. MS (ESI) m/z 419 [M+H]⁺.

### Example 3: 3-[4-[4-(dimethylaminomethyl)-1-[4-(dimethylaminomethyl)phenyl]-1H-pyrazol-3-yl]phenyl]-N,N-dimethyl-propan-1-amine (24)

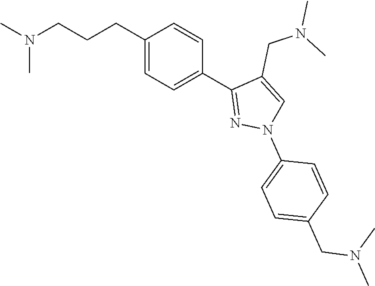

Compound 24 was obtained according to General procedure E from compound 20. 80% yield. The compound was converted to its 3HCl salt (white solid).

¹H NMR (free amine, CD₃OD, 300 Mz): δ 8.21 (s, 1H), 7.77 (d, J=8.6 Hz, 2H), 7.70 (d, J=8.2 Hz, 2H), 7.43 (d, J=8.6 Hz, 2H), 7.30 (t, J=8.2 Hz, 2H), 3.52 (s, 2H), 3.49 (s, 2H), 2.68 (t, J=7.6 Hz, 2H), 2.40-2.35 (m, 2H), 2.25 (s, 12H), 2.23 (s, 6H), 1.87-1.82 (m, 2H).

¹³C NMR (free amine, CD₃OD, 75 MHz): δ 154.0, 143.2, 140.5, 137.2, 132.2, 131.8, 130.4, 129.6, 129.6, 119.8, 118.8, 64.2, 60.1, 54.0, 45.4, 45.2, 45.1, 34.3, 30.0.

MS (ESI) m/z 420 [M+H]⁺.

P_{HPLC}>97%. HPLC (C₄, 35 min): t_{R} 8.4 min, P_{HPLC} 99%; HPLC (C₁₈, 35 min): t_{R} 10.8 min, P_{HPLC} 97%.

### Example 4: 3-[4-[1-[4-(dimethylaminomethyl)phenyl]-4-[3-(dimethylamino)propyl]-1H-pyrazol-3-yl]phenyl]-N,N-dimethyl-propan-1-amine (25)

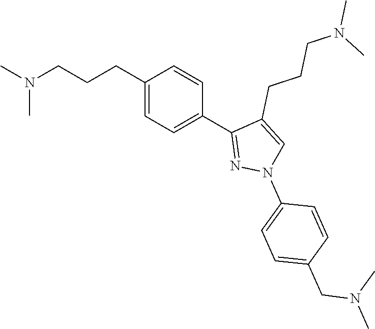

Compound 25 was obtained according to General procedure E from compound 21. 89% yield. The compound was converted to its 3HCl salt (white solid).

¹H NMR (free amine, CD₃OD, 300 Mz): δ 8.13 (s, 1H), 7.76 (d, J=8.5 Hz, 2H), 7.63 (d, J=8.1 Hz, 2H), 7.41 (d, J=8.5 Hz, 2H), 7.30 (d, J=8.1 Hz, 2H), 3.50 (s, 2H), 2.72-2.65 (m, 4H), 2.48-2.40 (m, 4H), 2.31 (s, 6H), 2.26 (s, 6H), 2.25 (s, 6H), 1.92-1.76 (m, 4H).

¹³C NMR (free amine, CD₃OD, 75 MHz): δ 152.7, 142.8, 140.7, 136.7, 132.6, 131.8, 129.6, 129.1, 128.4, 122.1, 119.6, 64.1, 60.0, 59.9, 45.2 (3C), 34.2, 29.7, 28.5, 23.3.

MS (ESI) m/z 448 [M+H]⁺.

P_{HPLC}>96%. HPLC (C₄, 35 min): t_{R} 8.7 min, P_{HPLC} 99%; HPLC (C₁₈, 35 min): t_{R} 11.8 min, P_{HPLC} 96%.

### Example 5: 3-[4-[4-(dimethylaminomethyl)-1-[3-(dimethylaminomethyl)phenyl]pyrazol-3-yl]phenyl]-N,N-dimethyl-propan-1-amine (26)

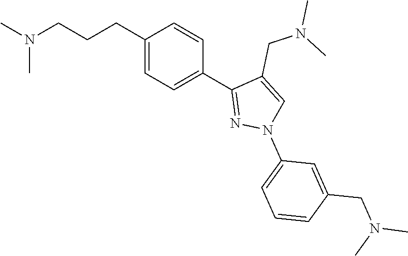

Compound 26 was obtained according to General procedure E from compound 22. 72% yield. The compound was converted to its 3HCl salt (white solid).

¹H NMR (free amine, CDCl₃, 300 Mz): δ 7.97 (s, 1H), 7.79 (d, J=8.2 Hz, 2H), 7.71 (s, 1H), 7.67 (d, J=8.0 Hz, 1H), 7.37 (t, J=7.7 Hz, 1H), 7.25 (d, J=8.3 Hz, 2H), 7.20 (d, J=7.6 Hz, 1H), 3.46 (s, 2H), 3.46 (s, 2H), 2.68 (t, J=7.6 Hz, 2H), 2.37 (t, J=7.2 Hz, 2H), 2.28 (s, 6H), 2.27 (s, 6H), 2.25 (s, 6H), 1.86 (quint, J=7.4 Hz, 2H).

¹³C NMR (CDCl₃, 75 MHz): δ 152.2, 141.6, 140.6, 140.1, 131.1, 129.3, 128.5, 128.3, 128.1, 126.7, 119.1, 118.4, 117.6, 64.2, 59.1, 53.9, 45.5, 45.2, 45.2, 33.4, 29.0.

MS (ESI) m/z 420 [M+H]⁺.

P_{HPLC}>97%. HPLC (C₄, 35 min): t_{R} 7.6 min, P_{HPLC} 98%; HPLC (C₁₈, 35 min): t_{R} 11.1 min, P_{HPLC} 97%.

### Example 6: 3-[4-[1-[3-(dimethylaminomethyl)phenyl]-4-[3-(dimethylamino)propyl]pyrazol-3-yl]phenyl]-N,N-dimethyl-propan-1-amine (27)

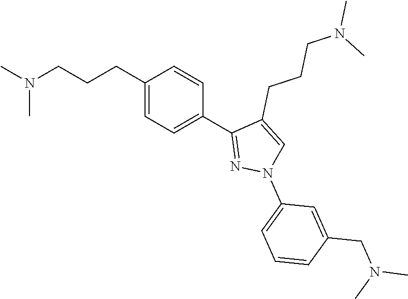

Compound 27 was obtained according to General procedure E from compound 23. 68% yield. The compound was converted to its 3HCl salt (white solid).

¹H NMR (free amine, CD₃OD, 300 Mz): δ 8.12 (s, 1H), 7.77 (s, 1H), 7.73-7.69 (m, 1H), 7.63 (d, J=8.2 Hz, 2H), 7.43 (t, J=7.8 Hz, 1H), 7.29 (d, J=8.1 Hz, 2H), 7.23 (d, J=7.6 Hz, 1H), 3.52 (s, 2H), 2.72-2.65 (m, 4H), 2.42-2.34 (m, 4H), 2.26 (s, 12H), 2.22 (s, 6H), 1.90-1.77 (m, 4H).

¹³C NMR (free amine, CD₃OD, 75 MHz): δ 152.8, 143.0, 141.4, 140.7, 132.6, 130.5, 129.6, 129.1, 128.4, 128.3, 122.3, 120.7, 118.8, 64.7, 60.2, 60.1, 45.3 (3C), 34.3, 29.9, 28.8, 23.4.

MS (ESI) m/z 448 [M+H]⁺.

P_{HPLC}>98%. HPLC (C₄, 35 min): t_{R} 8.9 min, P_{HPLC} 98%; HPLC (C₁₈, 35 min): t_{R} 12.1 min, P_{HPLC} 98%.

### Example 7: 3-[4-[4-(dimethylaminomethyl)-1-[4-[(4-methylpiperazin-1-yl)methyl]phenyl]-1H-pyrazol-3-yl]phenyl]-N,N-dimethyl-propan-1-amine (28)

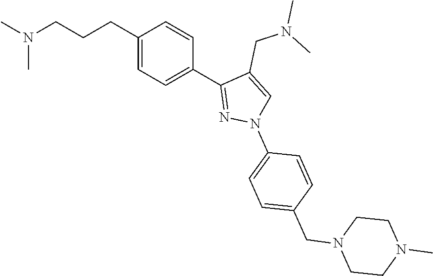

Compound 28 was obtained according to General procedure E from compound 20. 75% yield. The compound was converted to its 4HCl salt (white solid).

¹H NMR (free amine, CD₃OD, 300 Mz): δ 8.21 (s, 1H), 7.76 (d, J=8.6 Hz, 2H), 7.70 (d, J=8.2 Hz, 2H), 7.44 (d, J=8.6 Hz, 2H), 7.30 (t, J=8.2 Hz, 2H), 3.55 (s, 2H), 3.52 (s, 2H), 2.68 (t, J=7.6 Hz, 2H), 2.49 (br, 8H), 2.42-2.37 (m, 2H), 2.26 (s, 9H), 2.24 (s, 6H), 1.90-1.80 (m, 2H).

¹³C NMR (free amine, CD₃OD, 75 MHz): δ 153.9, 143.2, 140.4, 137.1, 132.2, 131.7, 130.4, 129.6, 129.6, 119.8, 118.7, 63.1, 60.1, 55.7, 54.0, 53.5, 46.0, 45.3, 45.1, 34.3, 29.9.

MS (ESI) m/z 475 [M+H]⁺.

P_{HPLC}>97%. HPLC (C₄, 35 min): t_{R} 7.6 min, P_{HPLC} 98%; HPLC (C₁₈, 35 min): t_{R} 10.5 min, P_{HPLC} 97%.

### Example 8: 3-[4-[4-[3-(dimethylamino)propyl]-1-[4-[(4-methylpiperazin-1-yl)methyl]phenyl]-1H-pyrazol-3-yl]phenyl]-N,N-dimethyl-propan-1-amine (29)

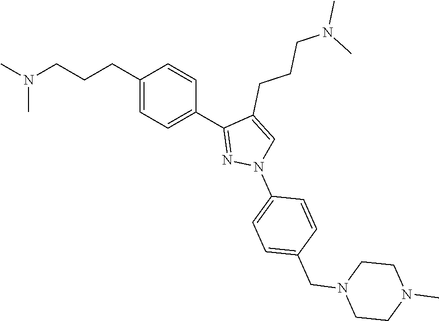

Compound 29 was obtained according to General procedure E from compound 21. 77% yield. The compound was converted to its 4HCl salt (white solid).

¹H NMR (free amine, CD₃OD, 300 Mz): δ 8.10 (s, 1H), 7.74 (d, J=8.5 Hz, 2H), 7.63 (d, J=8.1 Hz, 2H), 7.42 (d, J=8.5 Hz, 2H), 7.30 (d, J=8.1 Hz, 2H), 3.53 (s, 2H), 2.71-2.65 (m, 4H), 2.49 (br, 8H), 2.42-2.34 (m, 4H), 2.27 (s, 6H), 2.26 (s, 3H), 2.22 (s, 6H), 1.90-1.74 (m, 4H).

¹³C NMR (free amine, CD₃OD, 75 MHz): δ 152.7, 143.0, 140.6, 136.7, 132.6, 131.6, 129.6, 129.1, 128.4, 122.2, 119.6, 63.1, 60.1, 60.0, 55.7, 53.5, 46.0, 45.3 (2C), 34.3, 29.9, 28.7, 23.4.

MS (ESI) m/z 503 [M+H]⁺.

P_{HPLC}>97%. HPLC (C₄, 35 min): t_{R} 6.6 min, P_{HPLC} 98%; HPLC (C₁₈, 35 min): t_{R} 11.3 min, P_{HPLC} 97%.

### Example 9: 3-[4-[4-(dimethylaminomethyl)-1-[3-[(4-methylpiperazin-1-yl)methyl]phenyl]-1H-pyrazol-3-yl]phenyl]-N,N-dimethyl-propan-1-amine (30)

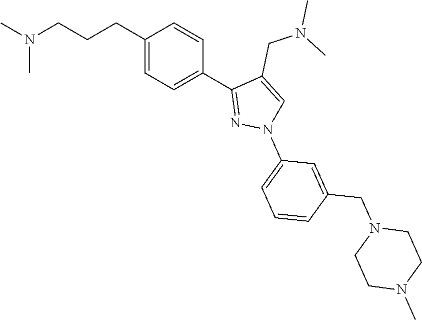 [SMILES: CN(C)CCCc1ccc(-c2nn(-c3cccc(CN4CCN(C)CC4)c3)cc2CN(C)C)cc1]

Compound 30 was obtained according to General procedure E from compound 22. 74% yield. The compound was converted to its 4HCl salt (white solid).

¹H NMR (free amine, CD₃OD, 300 Mz): δ 8.23 (s, 1H), 7.81 (s, 1H), 7.73-7.69 (m, 3H), 7.43 (t, J=7.8 Hz, 1H), 7.31 (d, J=8.1 Hz, 2H), 7.27 (d, J=8.0 Hz, 1H), 3.58 (s, 2H), 3.52 (s, 2H), 2.68 (t, J=7.6 Hz, 2H), 2.49-2.43 (m, 10H), 2.32 (s, 6H), 2.26 (s, 3H), 2.24 (s, 6H), 1.92-1.82 (m, 2H).

¹³C NMR (free amine, CD₃OD, 75 MHz): δ 153.9, 143.0, 141.3, 140.6, 132.2, 130.5, 130.4, 129.6, 129.6, 128.5, 120.8, 118.9, 118.7, 63.4, 59.9, 55.7, 54.0, 53.5, 45.9, 45.2, 45.1, 34.2, 29.7.

MS (ESI) m/z 475 [M+H]⁺.

P_{HPLC}>97%. HPLC (C₄, 35 min): t_{R} 5.1 min, P_{HPLC} 97%; HPLC (C₁₈, 35 min): t_{R} 10.9 min, P_{HPLC} 98%.

### Example 10: 3-[4-[4-[3-(dimethylamino)propyl]-1-[3-[(4-methylpiperazin-1-yl)methyl]phenyl-1H-pyrazol-3-yl]phenyl]-N,N-dimethyl-propan-1-amine (31)

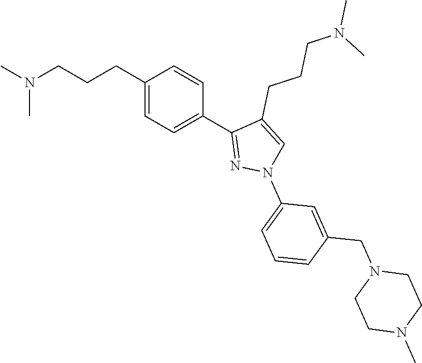 [SMILES: CN(C)CCCc1ccc(-c2nn(-c3cccc(CN4CCN(C)CC4)c3)cc2CCCN(C)C)cc1]

Compound 31 was obtained according to General procedure E from compound 23. 82% yield. The compound was converted to its 4HCl salt (white solid).

¹H NMR (free amine, CD₃OD, 300 Mz): δ 8.12 (s, 1H), 7.78 (s, 1H), 7.70-7.67 (m, 1H), 7.63 (d, J=8.1 Hz, 2H), 7.41 (t, J=7.8 Hz, 1H), 7.30 (d, J=8.1 Hz, 2H), 7.24 (d, J=7.6 Hz, 1H), 3.56 (s, 2H), 2.72-2.65 (m, 4H), 2.50 (br, 8H), 2.43-2.35 (m, 4H), 2.27 (s, 6H), 2.25 (s, 3H), 2.23 (s, 6H), 1.90-1.77 (m, 4H).

¹³C NMR (free amine, CD₃OD, 75 MHz): δ 152.8, 142.9, 141.4, 140.5, 132.6, 130.5, 129.6, 129.1, 128.4, 128.2, 122.2, 120.6, 118.7, 63.5, 60.1, 60.0, 55.7, 53.6, 46.0, 45.3 (2C), 34.3, 29.9, 28.7, 23.4.

MS (ESI) m/z 503 [M+H]⁺.

P_{HPLC}>97%. HPLC (C₄, 35 min): t_{R} 7.9 min, P_{HPLC} 97%; HPLC (C₁₈, 35 min): t_{R} 11.6 min, P_{HPLC} 97%.

### Example 11: N′-[[4-[4-(dimethylaminomethyl)-3-[4-[3-(dimethylamino)propyl]phenyl]-1H-pyrazol-1-yl]phenyl]methyl]-N,N,N′-trimethyl-propane-1,3-diamine (32)

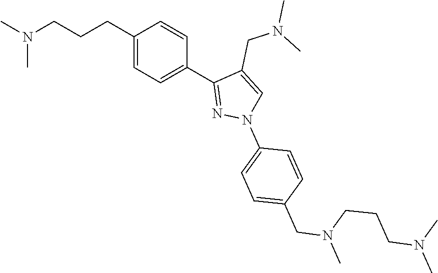

Compound 32 was obtained according to General procedure E from compound 20. 62% yield. The compound was converted to its 4HCl salt (white solid).

¹H NMR (free amine, CD₃OD, 300 Mz): δ 8.23 (s, 1H), 7.78 (d, J=8.5 Hz, 2H), 7.71 (d, J=8.2 Hz, 2H), 7.46 (d, J=8.5 Hz, 2H), 7.32 (t, J=8.1 Hz, 2H), 3.57 (s, 2H), 3.55 (s, 2H), 2.70 (t, J=7.6 Hz, 2H), 2.52-2.44 (m, 6H), 2.36 (s, 6H), 2.34 (s, 6H), 2.25 (s, 9H), 1.94-1.83 (m, 2H), 1.82-1.72 (m, 2H).

¹³C NMR (free amine, CD₃OD, 75 MHz): δ 154.0, 143.0, 140.4, 137.8, 132.2, 131.6, 130.5, 129.7, 129.6, 119.9, 118.6, 62.4, 59.9, 58.6, 56.0, 53.9, 45.1, 45.1, 45.1, 42.3, 34.2, 29.6, 25.2.

MS (ESI) m/z 491 [M+H]⁺.

P_{HPLC}>95%. HPLC (C₄, 35 min): t_{R} 8.1 min, P_{HPLC} 99%; HPLC (C₁₈, 35 min): t_{R} 10.3 min, P_{HPLC} 95%.

### Example 12: N′-[[4-[4-[3-(dimethylamino)propyl]-3-[4-[3-(dimethylamino)propyl]phenyl]-1H-pyrazol-1-yl]phenyl]methyl]-N,N,N′-trimethyl-propane-1,3-diamine (33)

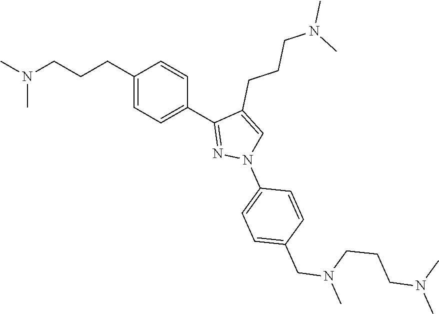

Compound 33 was obtained according to General procedure E from compound 21. 41% yield. The compound was converted to its 4HCl salt (white solid).

¹H NMR (free amine, CD₃OD, 300 Mz): δ 8.11 (s, 1H), 7.74 (d, J=8.6 Hz, 2H), 7.63 (d, J=8.2 Hz, 2H), 7.43 (d, J=8.6 Hz, 2H), 7.30 (d, J=8.2 Hz, 2H), 3.54 (s, 2H), 2.72-2.65 (m, 4H), 2.44-2.33 (m, 8H), 2.25 (s, 12H), 2.22 (s, 3H), 2.20 (s, 6H), 1.90-1.67 (m, 6H).

¹³C NMR (free amine, CD₃OD, 75 MHz): δ 152.8, 143.0, 140.5, 137.5, 132.6, 131.6, 129.6, 129.1, 128.4, 122.3, 119.6, 62.4, 60.2, 60.1, 58.6, 56.2, 45.4 (3C), 42.3, 34.3, 30.0, 28.8, 25.7, 23.4.

MS (ESI) m/z 519 [M+H]⁺.

P_{HPLC}>97%. HPLC (C₄, 35 min): t_{R} 8.3 min, P_{HPLC} 97%; HPLC (C₁₈, 35 min): t_{R} 11.3 min, P_{HPLC} 97%.

### Example 13: N′-[[3-[4-(dimethylaminomethyl)-3-[4-[3-(dimethylamino)propyl]phenyl]-1H-pyrazol-1-yl]phenyl]methyl]-N,N,N′-trimethyl-propane-1,3-diamine (34)

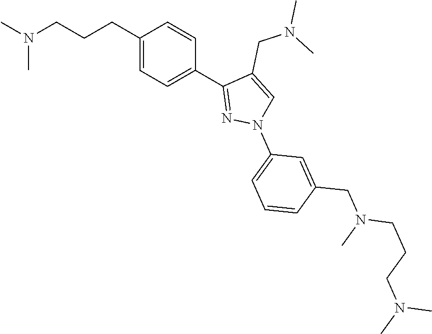

Compound 34 was obtained according to General procedure E from compound 22. 60% yield. The compound was converted to its 4HCl salt (white solid).

¹H NMR (free amine, CD₃OD, 300 Mz): δ 8.22 (s, 1H), 7.81 (s, 1H), 7.73-7.68 (m, 3H), 7.43 (t, J=7.8 Hz, 1H), 7.29 (d, J=8.3 Hz, 2H), 7.27 (d, J=7.8 Hz, 1H), 3.58 (s, 2H), 3.52 (s, 2H), 2.67 (t, J=7.6 Hz, 2H), 2.45-2.32 (m, 6H), 2.24-2.22 (m, 21H), 1.89-1.69 (m, 4H).

¹³C NMR (free amine, CD₃OD, 75 MHz): δ 153.9, 143.2, 141.6, 141.3, 132.2, 130.5, 130.3, 129.6, 129.5, 128.4, 120.7, 118.8, 118.7, 62.9, 60.1, 58.6, 56.3, 54.0, 45.4 (2C), 45.2, 42.5, 34.4, 30.0, 25.8.

MS (ESI) m/z 491 [M+H]⁺.

P_{HPLC}>96%. HPLC (C₄, 35 min): t_{R} 8.0 min, P_{HPLC} 96%; HPLC (C₁₈, 35 min): t_{R} 10.6 min, P_{HPLC} 96%.

### Example 14: N′-[[3-[4-[3-(dimethylamino)propyl]-3-[4-[3-(dimethylamino)propyl]phenyl]-1H-pyrazol-1-yl]phenyl]methyl]-N,N,N′-trimethyl-propane-1,3-diamine (35)

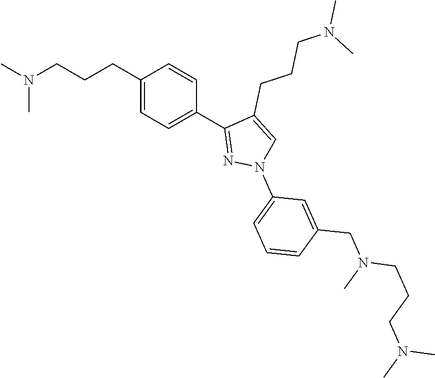

Compound 35 was obtained according to General procedure E from compound 23. 62% yield. The compound was converted to its 4HCl salt (white solid).

¹H NMR (free amine, CD₃OD, 300 Mz): δ 8.12 (s, 1H), 7.79 (s, 1H), 7.70-7.67 (m, 1H), 7.63 (d, J=8.1 Hz, 2H), 7.42 (t, J=7.8 Hz, 1H), 7.30 (d, J=8.1 Hz, 2H), 7.24 (d, J=7.6 Hz, 1H), 3.57 (s, 2H), 2.72-2.65 (m, 4H), 2.45-2.31 (m, 8H), 2.23 (s, 9H), 2.21 (s, 6H), 2.19 (s, 6H), 1.89-1.67 (m, 6H).

¹³C NMR (free amine, CD₃OD, 75 MHz): δ 152.8, 143.1, 141.5, 141.4, 132.7, 130.4, 129.6, 129.1, 128.4, 128.1, 122.3, 120.6, 118.5, 62.9, 60.2, 60.1, 58.6, 56.3, 45.4 (3C), 42.5, 34.4, 30.1, 28.9, 25.8, 23.5.

MS (ESI) m/z 519 [M+H]⁺.

P_{HPLC}>97%. HPLC (C₄, 35 min): t_{R} 7.5 min, P_{HPLC} 98%; HPLC (C₁₈, 35 min): t_{R} 11.4 min, P_{HPLC} 97%.

### Methyl 3-[2-[1-(4-cyanophenyl)ethylidene]hydrazino]benzoate (38)

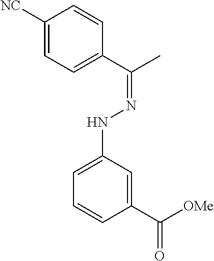 [SMILES: COC(=O)c1cccc(N/N=C(/C)c2ccc(C#N)cc2)c1]

A mixture of 4′-cyanoacetophenone (5 g, 34.4 mmol) and 4 (10 g, 49.3 mmol) in methanol (90 mL) was refluxed for 6 h. The reaction mixture was then stirred at rt for 18 h. The solid was collected by filtration, washed with methanol and dried to give 7.34 g (73%) of the product as a yellow solid.

¹H NMR (CD₃SOCD₃, 300 Mz): δ 9.80 (s, 1H), 7.94 (d, J=8.6 Hz, 2H), 7.87-7.86 (m, 1H), 7.82 (d, J=8.6 Hz, 2H), 7.58-7.54 (m, 1H), 7.42-7.35 (m, 2H), 3.85 (s, 3H), 2.27 (s, 3H).

¹³C NMR (CD₃SOCD₃, 75 MHz): δ 166.5, 145.8, 143.3, 139.7, 132.2, 130.4, 129.4, 125.8, 120.1, 119.1, 117.4, 113.8, 109.5, 52.1, 12.6.

MS (ESI) m/z 294 [M+H]⁺.

### Methyl 3-[2-[1-(3-cyanophenyl)ethylidene]hydrazino]benzoate (39)

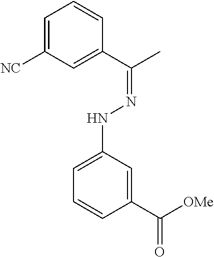 [SMILES: COC(=O)c1cccc(N/N=C(/C)c2cccc(C#N)c2)c1]

A solution of 3′-cyanoacetophenone (3.5 g, 24.1 mmol) and 4 (5.8 g, 28.6 mmol) in methanol (30 mL) was stirred at rt for 1 h, heated at reflux for 6 h and then stirred at rt for 18 h. The solid was collected by filtration, washed with methanol and dried to give 5.29 g (75%) of the product as a white solid.

¹H NMR (CD₃SOCD₃, 300 Mz): δ 9.71 (s, 1H), 8.16-8.10 (m, 2H), 7.84 (dd, J=1.1, 1.1 Hz, 1H), 7.75 (ddd, J=7.7, 1.2, 1.2 Hz, 1H), 7.62-7.56 (m, 2H), 7.39-7.38 (m, 2H), 3.85 (s, 3H), 2.29 (s, 3H).

¹³C NMR (CD₃SOCD₃, 75 MHz): δ 166.5, 145.9, 140.2, 139.7, 131.0, 130.3, 129.7, 129.6, 129.5, 128.7, 119.9, 118.9, 117.3, 113.7, 111.6, 52.1, 12.8.

MS (ESI) m/z 292 [M−H]⁺.

### Methyl 3-[3-(4-cyanophenyl)-4-formyl-pyrazol-1-yl]benzoate (40)

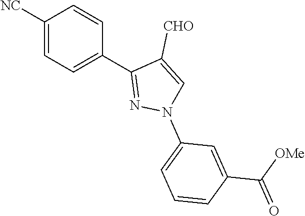 [SMILES: COC(=O)c1cccc(-n2cc(C=O)c(-c3ccc(C#N)cc3)n2)c1]

Dimethylformamide (26.9 mL, 348 mmol) was cooled to −5°C with a salt/ice bath. Phosphorus oxychloride (15.3 g, 9.28 mL, 99.6 mmol) was added dropwise while maintaining the temperature below 0°C. The mixture was stirred at −5°C for 40 min and 38 (7.3 g, 24.9 mmol) was slowly added. The reaction mixture was allowed to warm to rt. After 1 h, the mixture was heated at 50°C for 4 h. The mixture was then poured on water and stirred for 2 h. The solid was collected by filtration, washed with a mixture of methanol and ether (1/3) and dried to give 7.84 g (95%) of the product as a white solid.

¹H NMR (CF₃COOD, 300 Mz): δ 69.90 (s, 1H), 9.81 (s, 1H), 8.44 (s, 1H), 8.20 (d, J=7.7 Hz, 1H), 7.97-7.94 (m, 3H), 7.86 (d, J=7.9 Hz, 2H), 7.70-7.65 (m, 1H), 4.04 (s, 3H).

¹³C NMR (CF₃COOD, 75 MHz): δ 190.7, 171.2, 156.9, 140.4, 138.7, 136.6, 135.1, 133.5, 133.2, 132.7, 132.2, 128.6, 124.9, 123.8, 55.3.

MS (ESI) m/z 332 [M+H]⁺.

### Methyl 3-[3-(3-cyanophenyl)-4-formyl-pyrazol-1-yl]benzoate (41)

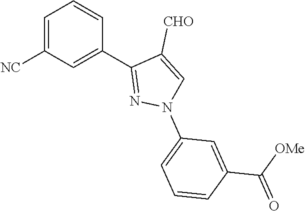 [SMILES: COC(=O)c1cccc(-n2cc(C=O)c(-c3cccc(C#N)c3)n2)c1]

Compound 41 was obtained according to General procedure A from compound 39. 95% yield (white solid).

¹H NMR (CDCl₃, 300 Mz): δ 10.07 (s, 1H), 8.64 (s, 1H), 8.43 (dd, J=1.8, 1.8 Hz, 1H), 8.29 (dd, J=1.4, 1.4 Hz, 1H), 8.22 (ddd, J=7.9, 1.3, 1.3 Hz, 1H), 8.09 (ddd, J=7.9, 1.3, 1.3 Hz, 1H), 8.07-8.03 (m, 1H), 7.75 (ddd, J=7.8, 1.6, 1.6 Hz, 1H), 7.66-7.59 (m, 2H), 3.99 (s, 3H).

¹³C NMR (CDCl₃, 75 MHz): δ 183.6, 165.9, 151.8, 139.0, 133.6, 133.3, 132.8, 132.7, 132.5, 132.1, 130.2, 129.6, 129.3, 124.0, 123.1, 120.4, 118.6, 113.1, 52.8.

MS (ESI) m/z 332 [M+H]⁺.

### Methyl 3-[3-(4-cyanophenyl)-4-methyl-pyrazol-1-yl]benzoate (42)

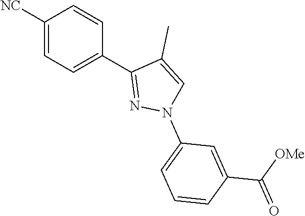 [SMILES: COC(=O)c1cccc(-n2cc(C)c(-c3ccc(C#N)cc3)n2)c1]

A mixture of 40 (7.76 g, 23.4 mmol), triethylsilane (9.45 mL, 58.5 mmol), and trifluoroacetic acid (26.1 mL, 351 mmol) was stirred vigorously at rt for 24 h. The reaction mixture was then evaporated to dryness. The residue was purified by column chromatography (DCM/MeOH=95:5) to give 6.44 g (87%) of the product as a white solid.

¹H NMR (CDCl₃, 300 Mz): δ 8.34-8.33 (m, 1H), 8.01-7.93 (m, 4H), 7.89-7.88 (m, 1H), 7.75-7.72 (m, 2H), 7.54 (dd, J=8.0, 8.0 Hz, 1H), 3.96 (s, 3H), 2.35 (s, 3H).

¹³C NMR (CDCl₃, 75 MHz): δ 166.4, 149.9, 140.1, 138.3, 132.5, 131.7, 129.8, 127.9, 127.8, 127.5, 123.1, 119.4, 119.1, 117.4, 111.2, 52.6, 10.6.

MS (ESI) m/z 318 [M+H]⁺.

### Methyl 3-[3-(3-cyanophenyl)-4-methyl-pyrazol-1-yl]benzoate (43)

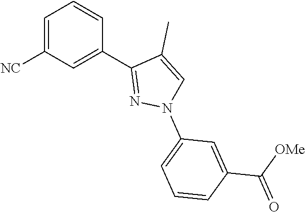 [SMILES: COC(=O)c1cccc(-n2cc(C)c(-c3cccc(C#N)c3)n2)c1]

A mixture of 41 (2.5 g, 7.55 mmol), triethylsilane (2.1 g, 2.92 mL, 18.1 mmol) and trifluoroacetic acid (12.8 g, 8.33 mL, 112 mmol) was vigorously stirred at rt for 24 h. It was then evaporated to dryness. The residue was purified by column chromatography (DCM) to give 2.07 g (86%) of the product as a white solid.

¹H NMR (CDCl₃, 300 Mz): δ 8.33 (s, 1H), 8.11 (s, 1H), 8.05 (d, J=7.8 Hz, 1H), 8.00-7.94 (m, 2H), 7.88 (s, 1H), 7.64 (d, J=7.7 Hz, 1H), 7.58-7.52 (m, 2H), 3.97 (s, 3H), 2.34 (s, 3H).

¹³C NMR (CDCl₃, 75 MHz): δ 166.3, 149.5, 139.9, 134.9, 131.5, 131.5, 131.0, 130.8, 129.7, 129.4, 127.6, 127.3, 122.9, 119.2, 118.9, 116.9, 112.7, 52.4, 10.4.

MS (ESI) m/z 318 [M+H]⁺.

### Methyl 3-[3-[4-(aminomethyl)phenyl]-4-methyl-pyrazol-1-yl]benzoate (44)

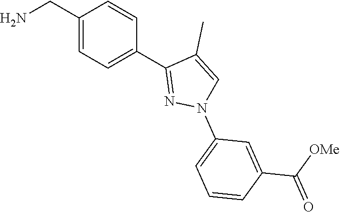 [SMILES: COC(=O)c1cccc(-n2cc(C)c(-c3ccc(CN)cc3)n2)c1]

To a solution of 42 (6.37 g, 20.1 mmol) in anhydrous THF (90 mL) under nitrogen was added BH₃-THF (1 M in THF, 30.1 mL, 30.1 mmol). The reaction mixture was refluxed for 3 h. Methanol (25 mL) was then slowly added. HCl (4 M in dioxane, 27.6 mL, 110 mmol) was then added and the mixture was refluxed for 90 min. The solvent was evaporated. Ethyl acetate and water were added to the residue and the pH of the mixture was brought to 10 by the addition of potassium carbonate. The layers were separated. The aqueous layer was extracted twice with ethyl acetate. The combined organic layers were washed with brine, dried, and evaporated. The residue was purified by column chromatography (DCM/MeOH—NH₃ sat=95:5) to give 3.3 g (51%) of the product as a colorless oil.

¹H NMR (CDCl₃, 300 Mz): δ 8.26-8.25 (m, 1H), 7.89-7.85 (m, 1H), 7.82-7.79 (m, 1H), 7.71-7.68 (m, 3H), 7.38 (dd, J=7.9, 7.9 Hz, 1H), 7.31 (d, J=8.2 Hz, 2H), 3.85 (s, 3H), 3.82 (s, 2H), 2.21 (s, 3H), 1.53 (s, 2H).

¹³C NMR (CDCl₃, 75 MHz): δ 166.1, 151.5, 142.7, 139.9, 131.8, 131.1, 129.2, 127.4, 126.9, 126.8, 126.4, 122.4, 118.6, 116.4, 52.1, 46.0, 10.1.

MS (ESI) m/z 322 [M+H]⁺.

### Methyl 3-[3-[3-(aminomethyl)phenyl]-4-methyl-pyrazol-1-yl]benzoate (45)

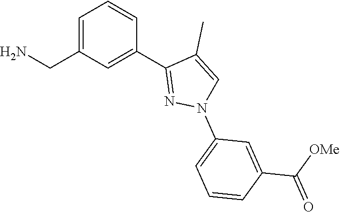 [SMILES: COC(=O)c1cccc(-n2cc(C)c(-c3cccc(CN)c3)n2)c1]

To a solution of 43 (3.03 g, 9.53 mmol) in anhydrous THF (60 mL) under nitrogen was added BH₃-THF (1 M in THF, 13.3 mL, 13.3 mmol). The reaction mixture was refluxed for 2 h 30. Methanol (25 mL) was then slowly added, followed by. HCl (4 M in dioxane, 13.1 mL, 52.4 mmol) and the mixture were refluxed for 90 min. The solvent was evaporated. Ethyl acetate and water were added to the residue and the pH of the mixture was brought to 10 by the addition of potassium carbonate. The layers were separated. The aqueous layer was extracted twice with ethyl acetate. The combined organic layers were washed with brine, dried and evaporated. The residue was purified by column chromatography (DCM/MeOH—NH₃ sat=95:5) to give 2.31 g (75%) of the product as a colorless oil.

¹H NMR (CDCl₃, 300 Mz): δ 8.32 (dd, J=1.8, 1.8 Hz, 1H), 7.98-7.95 (m, 1H), 7.89 (ddd, J=7.8, 1.3, 1.3 Hz, 1H), 7.81 (s, 1H), 7.75 (s, 1H), 7.62 (d, J=7.7 Hz, 1H), 7.48 (dd, J=7.9, 7.9 Hz, 1H), 7.40 (dd, J=7.6, 7.6 Hz, 1H), 7.30 (d, J=7.6 Hz, 1H), 3.92 (s, 5H), 2.29 (s, 3H), 1.69 (s, 2H).

¹³C NMR (CDCl₃, 75 MHz): δ 166.4, 152.0, 143.6, 140.2, 133.8, 131.4, 129.5, 128.7, 127.1, 126.8, 126.6, 126.3, 126.1, 122.9, 119.1, 116.8, 52.3, 46.6, 10.3.

MS (ESI) m/z 322 [M+H]⁺.

### Methyl 3-[3-[4-(dimethylaminomethyl)phenyl]-4-methyl-pyrazol-1-yl]benzoate (46)

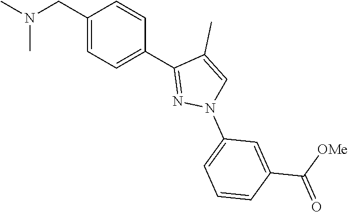 [SMILES: COC(=O)c1cccc(-n2cc(C)c(-c3ccc(CN(C)C)cc3)n2)c1]

Compound 46 was obtained according to General procedure B from compound 44. 46% yield (colorless oil).

¹H NMR (CD₃OD, 300 Mz): δ 8.24-8.22 (m, 1H), 7.83-7.83 (m, 1H), 7.82-7.78 (m, 1H), 7.74-7.71 (m, 1H), 7.64 (d, J=8.2 Hz, 2H), 7.37 (dd, J=7.9, 7.9 Hz, 1H), 7.28 (d, J=8.2 Hz, 2H), 3.82 (s, 3H), 3.38 (s, 2H), 2.27 (s, 6H), 2.14 (s, 3H).

¹³C NMR (CD₃OD, 75 MHz): δ 167.4, 152.5, 141.2, 138.2, 133.9, 132.4, 130.6, 128.7, 128.4, 127.5, 123.3, 119.8, 117.8, 65.5, 52.8, 45.2, 10.5.

MS (ESI) m/z 350 [M+H]⁺.

### Methyl 3-[3-[3-(dimethylaminomethyl)phenyl]-4-methyl-pyrazol-1-yl]benzoate (47)

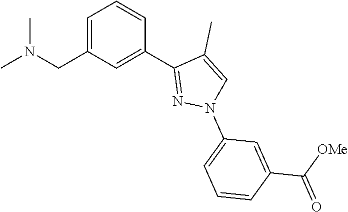 [SMILES: COC(=O)c1cccc(-n2cc(C)c(-c3cccc(CN(C)C)c3)n2)c1]

Compound 47 was obtained according to General procedure B from compound 45. 93% yield (colorless oil).

¹H NMR (CDCl₃, 300 Mz): δ 8.31 (dd, J=1.8, 1.8 Hz, 1H), 7.96-7.92 (m, 1H), 7.88-7.85 (m, 1H), 7.78-7.77 (m, 1H), 7.73-7.72 (m, 1H), 7.66-7.64 (m, 1H), 7.44 (dd, J=7.9, 7.9 Hz, 1H), 7.38 (dd, J=7.6, 7.6 Hz, 1H), 7.33-7.30 (m, 1H), 3.90 (s, 3H), 3.48 (s, 2H), 2.27-2.25 (m, 9H).

¹³C NMR (CDCl₃, 75 MHz): δ 166.3, 151.9, 140.1, 139.1, 133.4, 131.3, 129.4, 128.5, 128.4, 128.3, 126.9, 126.6, 126.3, 122.7, 118.9, 116.7, 64.3, 52.2, 45.3, 10.3.

MS (ESI) m/z 350 [M+H]⁺.

### Example 15: [3-[3-[4-(dimethylaminomethyl)phenyl]-4-methyl-pyrazol-1-yl]phenyl]methanol (48)

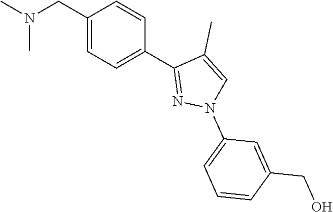 [SMILES: Cc1cn(-c2cccc(CO)c2)nc1-c1ccc(CN(C)C)cc1]

Compound 48 was obtained according to General procedure C from compound 46. 85% yield (colorless oil).

¹H NMR (CD₃OD, 300 Mz): δ 7.98-7.98 (m, 1H), 7.76-7.75 (m, 1H), 7.72-7.69 (m, 2H), 7.63-7.60 (m, 1H), 7.42-7.35 (m, 3H), 7.26-7.24 (m, 1H), 4.66 (s, 2H), 3.49 (s, 2H), 2.24 (s, 9H). ¹³C NMR (CD₃OD, 75 MHz): δ 152.5, 144.6, 141.3, 138.0, 134.2, 130.8, 130.5, 129.2, 128.6, 125.5, 118.4, 118.0, 117.5, 64.7, 64.5, 45.1, 10.3.

MS (ESI) m/z 322 [M+H]⁺.

### [3-[3-[3-(dimethylaminomethyl)phenyl]-4-methyl-pyrazol-1-yl]phenyl]methanol (49)

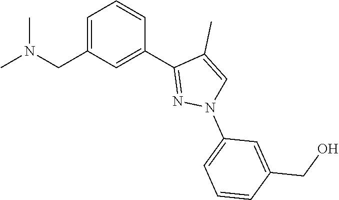 [SMILES: Cc1cn(-c2cccc(CO)c2)nc1-c1cccc(CN(C)C)c1]

Compound 49 was obtained according to General procedure C from compound 47. 88% yield (colorless oil).

¹H NMR (CDCl₃, 300 Mz): δ 7.72 (s, 1H), 7.67-7.64 (m, 3H), 7.58-7.55 (m, 1H), 7.39 (dd, J=7.6, 7.6 Hz, 1H), 7.33-7.28 (m, 2H), 7.13 (d, J=7.6 Hz, 1H), 4.62 (s, 2H), 4.48 (s, 1H), 3.48 (s, 2H), 2.26 (s, 3H), 2.23 (s, 6H).

¹³C NMR (CDCl₃, 75 MHz): δ 151.4, 143.2, 140.1, 138.4, 133.7, 129.3, 128.6, 128.5, 128.5, 127.2, 126.5, 124.1, 117.3, 116.7, 116.2, 64.2, 45.2 (2C), 10.3.

MS (ESI) m/z 322 ([M+H]⁺.

### 3-[3-[4-(dimethylaminomethyl)phenyl]-4-methyl-pyrazol-1-yl]benzaldehyde (50)

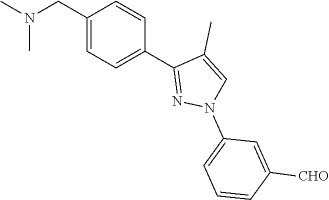 [SMILES: Cc1cn(-c2cccc(C=O)c2)nc1-c1ccc(CN(C)C)cc1]

Compound 50 was obtained according to General procedure D from compound 48. 56% yield (colorless oil).

¹H NMR (CDCl₃, 300 Mz): δ 9.99 (s, 1H), 8.15-8.14 (m, 1H), 7.99-7.95 (m, 1H), 7.78-7.77 (m, 1H), 7.72 (d, J=8.2 Hz, 2H), 7.68-7.65 (m, 1H), 7.52 (dd, J=7.8, 7.8 Hz, 1H), 7.36 (d, J=8.2 Hz, 2H), 3.44 (s, 2H), 2.26 (s, 3H), 2.23 (s, 6H).

¹³C NMR (CDCl₃, 75 MHz): δ 191.5, 152.0, 140.6, 138.4, 137.3, 132.2, 130.1, 129.3, 127.4, 126.9 (2C), 123.8, 118.3, 117.0, 64.0, 45.3, 10.3.

MS (ESI) m/z 320 [M+H]⁺.

### 3-[3-[3-(dimethylaminomethyl)phenyl]-4-methyl-pyrazol-1-yl]benzaldehyde (51)

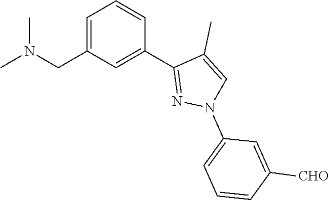 [SMILES: Cc1cn(-c2cccc(C=O)c2)nc1-c1cccc(CN(C)C)c1]

Compound 51 was obtained according to General procedure D from compound 49. 61% yield (colorless oil).

¹H NMR (CDCl₃, 300 Mz): δ 610.03 (s, 1H), 8.18 (dd, J=1.8, 1.8 Hz, 1H), 8.03-7.99 (m, 1H), 7.82-7.81 (m, 1H), 7.73 (dd, J=1.5, 1.5 Hz, 1H), 7.70 (ddd, J=7.6, 1.2, 1.2 Hz, 1H), 7.65 (ddd, J=7.5, 1.5, 1.5 Hz, 1H), 7.55 (dd, J=7.8, 7.8 Hz, 1H), 7.40 (dd, J=7.6, 7.6 Hz, 1H), 7.33 (ddd, J=7.7, 1.5, 1.5 Hz, 1H), 3.49 (s, 2H), 2.29-2.27 (m, 9H).

¹³C NMR (CDCl₃, 75 MHz): δ 191.6, 152.3, 140.8, 139.2, 137.5, 133.3, 130.1, 128.7, 128.5, 128.3, 127.0, 126.9, 126.4, 123.9, 118.5, 117.1, 64.4, 45.4, 10.3.

MS (ESI) m/z 320 [M+H]⁺.

### 3-(dimethylamino)propanoic acid hydrochloride (52)

A mixture of beta-alanine (5.4 g, 60.6 mmol), formic acid (40 mL, 1060 mmol) and 37% formaldehyde in water (13 mL, 173 mmol) was refluxed for 15 h. 37% HCl (12 mL) was added, and the reaction mixture was evaporated. The residue was washed with a mixture of ethyl acetate and methanol (4:1), collected by filtration, and dried to give 8.07 g (87%) of the product as a white solid.

¹H NMR (CD₃OD, 300 Mz): 83.44 (t, J=6.9 Hz, 2H), 2.93 (s, 6H), 2.88 (t, J=6.9 Hz, 2H).

¹³C NMR (CD₃OD, 75 MHz): δ 173.3, 54.6, 43.7, 29.8.

MS (ESI) m/z 117 [M]⁺.

### Methyl 3-[3-(4-{[3-(dimethylamino)propanamido]methyl}phenyl)-4-methyl-1H-pyrazol-1-yl]benzoate (53)

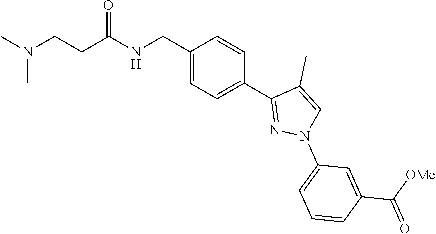 [SMILES: COC(=O)c1cccc(-n2cc(C)c(-c3ccc(CNC(=O)CCN(C)C)cc3)n2)c1]

To a mixture of 44 (1.87 g, 5.83 mmol), 52 (0.98 g, 6.41 mmol), hydroxybenzotriazole hydrate (1.02 g, 7.58 mmol) and triethylamine (3.25 mL, 23.3 mmol) in methylene chloride (60 mL) under nitrogen at rt was added 1-ethyl-3-(3-dimethylaminopropyl)carbodiimide hydrochloride (1.34 g, 7 mmol). The mixture was stirred for 20 h. 10% K₂CO₃ was added, and the layers were separated. The aqueous layer was extracted twice with methylene chloride. The combined organic layers were washed with brine, dried, and evaporated. The residue was purified by column chromatography (DCM/MeOH/NH₄OH=475:25:1) to give 1.77 g (72%) of the product as a white solid.

¹H NMR (CDCl₃, 300 Mz): 88.75 (br, 1H), 8.33-8.32 (m, 1H), 8.01-7.97 (m, 1H), 7.93-7.89 (m, 1H), 7.85-7.84 (m, 1H), 7.75 (d, J=8.2 Hz, 2H), 7.51 (dd, J=7.9, 7.9 Hz, 1H), 7.34 (d, J=8.2 Hz, 2H), 4.49 (d, J=5.7 Hz, 2H), 3.95 (s, 3H), 2.59 (t, J=5.9 Hz, 2H), 2.44 (t, J=6.2 Hz, 2H), 2.31 (s, 3H), 2.25 (s, 6H).

¹³C NMR (CDCl₃, 75 MHz): δ 172.6, 166.6, 151.8, 140.3, 138.5, 132.5, 131.5, 129.7, 127.9, 127.6, 127.2, 126.9, 123.0, 119.2, 116.9, 55.4, 52.5, 44.7, 42.9, 33.0, 10.4.

MS (ESI) m/z 421 [M+H]⁺.

### Methyl 3-[3-[3-[[3-(dimethylamino)propanoylamino]methyl]phenyl]-4-methyl-1H-pyrazol-1-yl]benzoate (54)

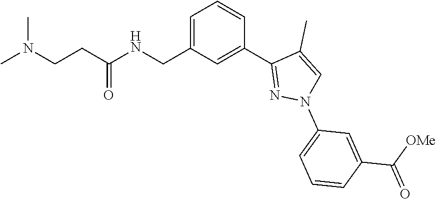 [SMILES: COC(=O)c1cccc(-n2cc(C)c(-c3cccc(CNC(=O)CCN(C)C)c3)n2)c1]

To a mixture of 45 (0.5 g, 1.56 mmol), 52 (0.26 g, 1.71 mmol), hydroxybenzotriazole hydrate (0.27 g, 2.02 mmol) and triethylamine (0.86 mL, 6.22 mmol) in methylene chloride (15 mL) under nitrogen at rt was added 1-ethyl-3-(3-dimethylaminopropyl)carbodiimide hydrochloride (0.36 g, 1.87 mmol). The mixture was stirred for 39 h. 10% K₂CO₃ was added, and the layers were separated. The aqueous layer was extracted with methylene chloride. The combined organic layers were washed with brine, dried, and evaporated. The residue was purified by column chromatography (DCM/MeOH/NH₄OH=95:5:0.3) to give 535 mg (82%) of the product as a colorless oil.

¹H NMR (CDCl₃, 300 Mz): δ 8.69-8.68 (m, 1H), 8.27 (dd, J=1.9, 1.9 Hz, 1H), 7.91-7.87 (m, 1H), 7.84 (ddd, J=7.8, 1.3, 1.3 Hz, 1H), 7.78-7.77 (m, 1H), 7.67 (s, 1H), 7.60 (d, J=7.7 Hz, 1H), 7.43 (dd, J=7.9, 7.9 Hz, 1H), 7.34 (dd, J=7.6, 7.6 Hz, 1H), 7.22 (d, J=7.7 Hz, 1H), 4.47 (d, J=5.7 Hz, 2H), 3.88 (s, 3H), 2.56 (t, J=6.0 Hz, 2H), 2.39 (t, J=6.3 Hz, 2H), 2.24 (s, 3H), 2.19 (s, 6H).

¹³C NMR (CDCl₃, 75 MHz): δ 172.3, 166.3, 151.7, 140.1, 139.1, 133.7, 131.3, 129.4, 128.6, 127.0, 126.7 (2C), 126.4, 126.2, 122.6, 118.9, 116.7, 55.1, 52.2, 44.5, 42.9, 32.9, 10.2.

MS (ESI) m/z 421 [M+H]⁺.

### (3-{3-[4-({[3-(dimethylamino)propyl]amino}methyl)phenyl]-4-methyl-1H-pyrazol-1-yl}phenyl)methanol (55)

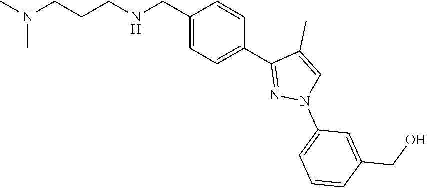 [SMILES: Cc1cn(-c2cccc(CO)c2)nc1-c1ccc(CNCCCN(C)C)cc1]

To a suspension of aluminum chloride (2.39 g, 18 mmol) in anhydrous THF (50 mL) at 0°C under nitrogen was added dropwise lithium aluminum hydride (1 M in THF, 18 mL, 18 mmol). The mixture was stirred for 20 min and a solution of 53 (1.68 g, 3.99 mmol) in anhydrous THF (50 mL) was added dropwise. The reaction mixture was stirred at 0°C for 30 min and then allowed to warm to rt. After 20 h, the solution was poured on ice. Ethyl acetate and K₂CO₃ were added, and the mixture was stirred for 15 min. The solid was filtered off. The layers were separated, and the organic layer was washed with brine, dried, and evaporated. The residue was purified by column chromatography (DCM/MeOH/NH₄OH=9:1:0.1) to give 740 mg (49%) of the product as a colorless oil.

¹H NMR (CDCl₃, 300 Mz): δ 7.75-7.70 (m, 4H), 7.61-7.58 (m, 1H), 7.40-7.34 (m, 3H), 7.22-7.19 (m, 1H), 4.69 (s, 2H), 3.79 (s, 2H), 3.00 (br, 2H), 2.66 (t, J=7.0 Hz, 2H), 2.30 (t, J=7.0 Hz, 2H), 2.27 (s, 3H), 2.20 (s, 6H), 1.68 (quint, J=7.2 Hz, 2H).

¹³C NMR (CDCl₃, 75 MHz): δ 151.5, 143.2, 140.3, 139.3, 132.6, 129.5, 128.4, 127.7, 127.2, 124.2, 117.5, 117.0, 116.3, 64.6, 58.2, 53.7, 47.9, 45.5, 27.6, 10.4.

MS (ESI) m/z 379 [M+H]⁺.

### [3-[3-[3-[[3-(dimethylamino)propylamino]methyl]phenyl]-4-methyl-1H-pyrazol-1-yl]phenyl]methanol (56)

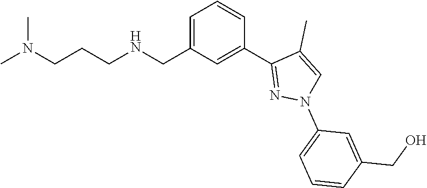 [SMILES: Cc1cn(-c2cccc(CO)c2)nc1-c1cccc(CNCCCN(C)C)c1]

To a suspension of aluminum chloride (1.53 g, 11.5 mmol) in anhydrous THF (40 mL) at 0°C under nitrogen was added dropwise LAH (1 M in THF, 11.5 mL, 11.5 mmol). The mixture was stirred for 20 min and a solution of 54 (1.07 g, 2.54 mmol) in anhydrous THF (40 mL) was added dropwise. The reaction mixture was stirred at 0°C for 30 min and then allowed to warm to rt. After 20 h, the solution was slowly poured on ice. Ethyl acetate and K₂CO₃ were added, and the mixture was stirred for 15 min. The solid was filtered off. The layers were separated, and the organic layer was washed with brine, dried, and evaporated. The residue was purified by column chromatography (DCM/MeOH/NH₄OH=9:1:0.1) to give 737 mg (77%) of the product as a colorless oil.

¹H NMR (CDCl₃, 300 Mz): δ 7.71-7.70 (m, 3H), 7.63-7.60 (m, 1H), 7.57-7.54 (m, 1H), 7.39-7.26 (m, 3H), 7.17 (d, J=7.7 Hz, 1H), 4.64 (s, 2H), 3.80 (s, 2H), 3.41 (br, 2H), 2.65 (t, J=7.0 Hz, 2H), 2.29 (t, J=7.1 Hz, 2H), 2.25 (s, 3H), 2.16 (s, 6H), 1.71-1.61 (m, 2H).

¹³C NMR (CDCl₃, 75 MHz): δ 151.4, 143.5, 140.2, 133.9, 129.3, 128.6, 127.5, 127.4, 127.2, 126.3, 124.1, 117.3, 116.8, 116.2, 64.2, 58.2, 53.9, 47.8, 45.4, 27.6, 10.3.

MS (ESI) m/z 379 [M+H]⁺.

### Example 16: (3-{3-[4-({[3-(dimethylamino)propyl](methyl)amino}methyl)phenyl]-4-methyl-1H-pyrazol-1-yl}phenyl)methanol (57)

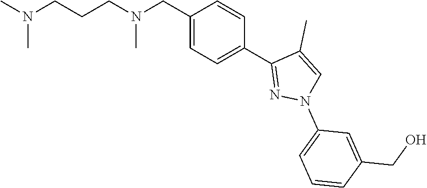 [SMILES: Cc1cn(-c2cccc(CO)c2)nc1-c1ccc(CN(C)CCCN(C)C)cc1]

To a mixture of 55 (0.72 g, 1.9 mmol), 37% formaldehyde in water (0.85 mL, 11.4 mmol) and acetic acid (0.65 mL, 11.4 mmol) in methanol (15 mL) was slowly added over 40 min STAB (2.42 g, 11.4 mmol). The mixture was stirred for 1 h and the solvent was evaporated. Ethyl acetate and 10% K₂CO₃ were added. After 10 min of stirring, the layers were separated. The aqueous layer was extracted twice with ethyl acetate. The combined organic layers were washed with brine, dried, and evaporated. The residue was purified by column chromatography (DCM/MeOH—NH₃ sat=95:5 to 92:8) to give 0.55 g (74%) of the product as a colorless oil.

¹H NMR (CDCl₃, 300 Mz): δ 7.77-7.70 (m, 4H), 7.63-7.60 (m, 1H), 7.42-7.35 (m, 3H), 7.23-7.21 (m, 1H), 4.72 (s, 2H), 3.51 (s, 2H), 2.92 (br, 1H), 2.40 (t, J=7.3 Hz, 2H), 2.33-2.28 (m, 5H), 2.22 (s, 6H), 2.20 (s, 3H), 1.76-1.66 (m, 2H).

¹³C NMR (CDCl₃, 75 MHz): δ 151.7, 143.0, 140.4, 138.6, 132.5, 129.6, 129.3, 127.5, 127.2, 124.2, 117.6, 117.0, 116.3, 64.8, 62.2, 57.9, 55.6, 45.5, 42.3, 25.6, 10.4.

MS (ESI) m/z 393 [M+H]⁺.

### Example 17: [3-[3-[3-[[(3-(dimethylamino)propyl)(methyl)amino]methyl]phenyl]-4-methyl-1H-pyrazol-1-yl]phenyl]methanol (58)

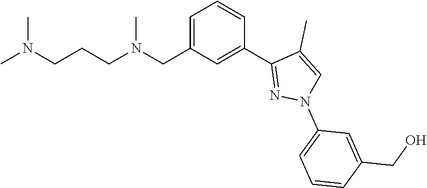 [SMILES: Cc1cn(-c2cccc(CO)c2)nc1-c1cccc(CN(C)CCCN(C)C)c1]

To a solution of 56 (0.79 g, 2.08 mmol), 37% formaldehyde in water (0.47 mL, 6.23 mmol) and acetic acid (0.36 mL, 6.23 mmol) in methanol (15 mL) was slowly added over 15 min STAB (1.1 g, 5.19 mmol). The mixture was stirred at rt for 15 h and the solvent was evaporated. Methylene chloride and water were added to the residue. The mixture was brought to pH=10 with ammonium hydroxide and the layers were separated. The aqueous layer was extracted twice with methylene chloride. The combined organic layers were washed with brine, dried, and evaporated. The residue was purified by column chromatography (DCM/MeOH/NH₄OH=95:5:0.5) to give 591 mg (73%) of the product as a colorless oil.

¹H NMR (CDCl₃, 300 Mz): δ 7.73-7.70 (m, 3H), 7.65-7.62 (m, 1H), 7.57-7.54 (m, 1H), 7.39-7.27 (m, 3H), 7.16 (d, J=7.6 Hz, 1H), 4.95 (br, 1H), 4.64 (s, 2H), 3.52 (s, 2H), 2.39 (t, J=7.2 Hz, 2H), 2.33-2.28 (m, 2H), 2.26 (s, 3H), 2.19 (s, 3H), 2.18 (s, 6H), 1.74-1.64 (m, 2H). ¹³C NMR (CDCl₃, 75 MHz): δ 151.5, 143.5, 140.1, 139.1, 133.6, 129.2, 128.3, 128.2, 127.1, 126.2, 124.0, 117.1, 116.7, 116.1, 64.1, 62.3, 57.7, 55.4, 45.2, 42.2, 25.2, 10.3.

MS (ESI) m/z 393 [M+H]⁺

### 3-{3-[4-({[3-(dimethylamino)propyl](methyl)amino}methyl)phenyl]-4-methyl-1H-pyrazol-1-yl}benzaldehyde (59)

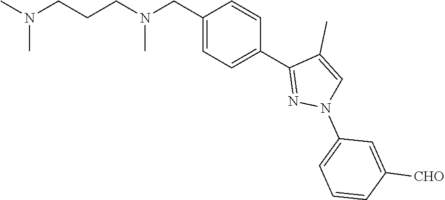 [SMILES: Cc1cn(-c2cccc(C=O)c2)nc1-c1ccc(CN(C)CCCN(C)C)cc1]

Compound 59 was obtained according to General procedure D from compound 57. 66% yield (colorless oil).

¹H NMR (CDCl₃, 300 Mz): δ 10.03 (s, 1H), 8.18-8.17 (m, 1H), 8.03-7.99 (m, 1H), 7.82-7.82 (m, 1H), 7.73-7.68 (m, 3H), 7.56 (dd, J=7.8, 7.8 Hz, 1H), 7.38 (d, J=8.2 Hz, 2H), 3.51 (s, 2H), 2.41 (t, J=7.3 Hz, 2H), 2.34-2.29 (m, 5H), 2.22 (s, 6H), 2.20 (s, 3H), 1.75-1.65 (m, 2H).

¹³C NMR (CDCl₃, 75 MHz): δ 191.6, 152.2, 140.8, 139.0, 137.5, 132.0, 130.2, 129.2, 127.4, 127.0, 126.9, 123.9, 118.4, 117.1, 62.2, 57.9, 55.5, 45.5, 42.3, 25.7, 10.4.

MS (ESI) m/z 391 [M+H]⁺.

### 3-[3-[3-[[3-(dimethylamino)propyl-methyl-amino]methyl]phenyl]-4-methyl-1H-pyrazol-1-yl]benzaldehyde (60)

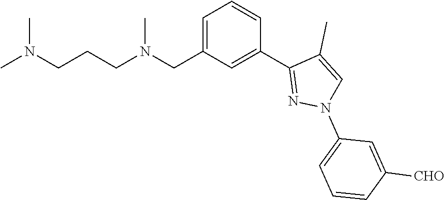 [SMILES: Cc1cn(-c2cccc(C=O)c2)nc1-c1cccc(CN(C)CCCN(C)C)c1]

Compound 60 was obtained according to General procedure D from compound 58. 55% yield (colorless oil).

¹H NMR (CDCl₃, 300 Mz): δ 10.00 (s, 1H), 8.15 (dd, J=1.7, 1.7 Hz, 1H), 8.00-7.96 (m, 1H), 7.79-7.78 (m, 1H), 7.71-7.70 (m, 1H), 7.67 (ddd, J=7.6, 1.2, 1.2 Hz, 1H), 7.62 (ddd, J=7.5, 1.5, 1.5 Hz, 1H), 7.52 (dd, J=7.9, 7.9 Hz, 1H), 7.36 (dd, J=7.6, 7.6 Hz, 1H), 7.31-7.29 (m, 1H), 3.52 (s, 2H), 2.41 (t, J=7.2 Hz, 2H), 2.32-2.27 (m, 2H), 2.27 (s, 3H), 2.19 (s, 9H), 1.73-1.63 (m, 2H).

¹³C NMR (CDCl₃, 75 MHz): δ 191.5, 152.3, 140.7, 139.6, 137.4, 133.2, 130.1, 128.5, 128.4, 128.1, 126.9, 126.2, 123.8, 118.3, 117.0, 62.3, 57.8, 55.6, 45.4, 42.2, 25.6, 10.3.

MS (ESI) m/z 391 [M+H]⁺.

### Example 18: {[4-(1-{3-[(dimethylamino)methyl]phenyl}-4-methyl-1H-pyrazol-3-yl)phenyl]methyl}dimethylamine (61)

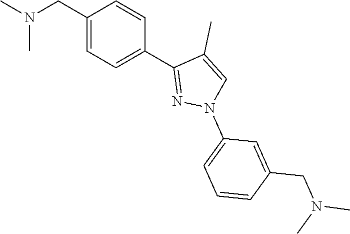

General procedure E. 79% yield. The compound was converted to its 2HCl salt (white solid).

¹H NMR (free amine, CD₃OD, 300 Mz): δ 8.04-8.03 (m, 1H), 7.74-7.72 (m, 3H), 7.69-7.65 (m, 1H), 7.43-7.37 (m, 3H), 7.22-7.20 (m, 1H), 3.49 (s, 2H), 3.48 (s, 2H), 2.28-2.24 (m, 15 H).

¹³C NMR (free amine, CD₃OD, 75 MHz): δ 152.6, 141.4, 140.7, 138.3, 134.2, 130.8, 130.5, 129.2, 128.6, 128.2, 120.6, 118.7, 117.6, 64.7, 64.6, 45.3, 45.2, 10.3.

MS (ESI) m/z 349 [M+H]⁺.

P_{HPLC}>96%. HPLC (C₄, 35 min): t_{R} 13.4 min, P_{HPLC} 99%; HPLC (C₁₈, 35 min): t_{R} 17.6 min, P_{HPLC} 96%.

### Example 19: dimethyl({[4-(4-methyl-1-{3-[(4-methylpiperazin-1-yl)methyl]phenyl}-1H-pyrazol-3-yl)phenyl]methyl})amine (62)

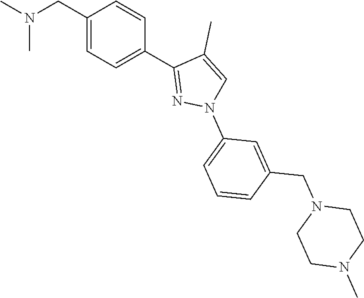 [SMILES: Cc1cn(-c2cccc(CN3CCN(C)CC3)c2)nc1-c1ccc(CN(C)C)cc1]

General procedure E. 61% yield. The compound was converted to its 3HCl salt (white solid).

¹H NMR (free amine, CD₃OD, 300 Mz): δ 8.04-8.04 (m, 1H), 7.76-7.71 (m, 3H), 7.67-7.64 (m, 1H), 7.42-7.38 (m, 3H), 7.24-7.22 (m, 1H), 3.56 (s, 2H), 3.50 (s, 2H), 2.49 (m, 8H), 2.28-2.25 (m, 12H).

¹³C NMR (free amine, CD₃OD, 75 MHz): δ 152.6, 141.4, 140.5, 138.3, 134.2, 130.8, 130.4, 129.2, 128.7, 128.2, 120.6, 118.7, 117.6, 64.6, 63.5, 55.7, 53.6, 46.0, 45.2, 10.3.

MS (ESI) m/z 404 [M+H]⁺.

P_{HPLC}>96%. HPLC (C₄, 30 min): t_{R} 12.1 min, P_{HPLC} 96%; HPLC (C₁₈, 30 min): t_{R} 17.3 min, P_{HPLC} 97%.

### Example 20: [(4-{1-[3-({[3-(dimethylamino)propyl](methyl)amino}methyl)phenyl]-4-methyl-1H-pyrazol-3-yl}phenyl)methyl]dimethylamine (63)

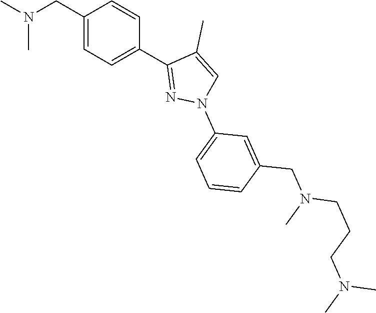

General procedure E. 73% yield. The compound was converted to its 3HCl salt (white solid).

¹H NMR (free amine, CD₃OD, 300 Mz): δ 8.05-8.05 (m, 1H), 7.77-7.72 (m, 3H), 7.67-7.64 (m, 1H), 7.43-7.38 (m, 3H), 7.24-7.21 (m, 1H), 3.56 (s, 2H), 3.50 (s, 2H), 2.44-2.39 (m, 2H), 2.36-2.31 (m, 2H), 2.29 (s, 3H), 2.25 (s, 6H), 2.23 (s, 3H), 2.22 (s, 6H), 1.76-1.66 (m, 2H).

¹³C NMR (free amine, CD₃OD, 75 MHz): δ 152.6, 141.4, 141.4, 138.3, 134.2, 130.8, 130.4, 129.2, 128.7, 128.1, 120.5, 118.5, 117.5, 64.7, 62.9, 58.6, 56.2, 45.4, 45.2, 42.5, 25.7, 10.4.

MS (ESI) m/z 420 [M+H]⁺.

P_{HPLC}>95%. HPLC (C₄, 35 min): t_{R} 12.7 min, P_{HPLC} 98%; HPLC (C₁₈, 35 min): t_{R} 16.6 min, P_{HPLC} 95%.

### Example 21: 1-[3-[1-[3-(dimethylaminomethyl)phenyl]-4-methyl-pyrazol-3-yl]phenyl]-N,N-dimethylmethanamine (64)

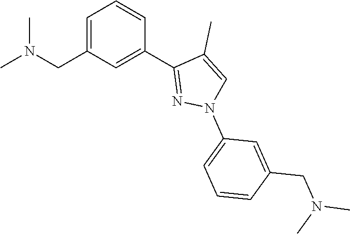

General procedure E. 80% yield. The compound was converted to its 2HCl salt (white solid).

¹H NMR (free amine, CD₃OD, 300 Mz): δ 8.05 (s, 1H), 7.76-7.72 (m, 2H), 7.70-7.66 (m, 2H), 7.45-7.39 (m, 2H), 7.33-7.30 (m, 1H), 7.23-7.21 (m, 1H), 3.53 (s, 2H), 3.51 (s, 2H), 2.29-2.25 (m, 15H).

¹³C NMR (free amine, CD₃OD, 75 MHz): δ 152.8, 141.4, 140.7, 139.0, 135.0, 130.5, 130.1, 129.9, 129.6, 129.2, 128.2, 127.9, 120.7, 118.8, 117.6, 64.9, 64.7, 45.3, 45.2, 10.3.

MS (ESI) m/z 349 [M+H]⁺.

P_{HPLC}>97%. HPLC (C₄, 35 min): t_{R} 9.1 min, P_{HPLC} 98%; HPLC (C₁₈, 35 min): t_{R} 13.7 min, P_{HPLC} 97%.

### Example 22: N,N-dimethyl-1-[3-[4-methyl-1-[3-[(4-methylpiperazin-1-yl)methyl]phenyl]-1H-pyrazol-3-yl]phenyl]methanamine (65)

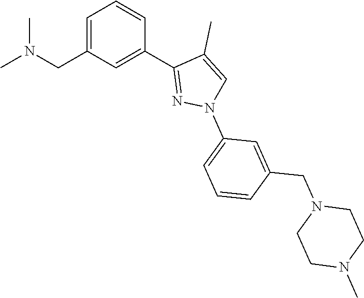 [SMILES: Cc1cn(-c2cccc(CN3CCN(C)CC3)c2)nc1-c1cccc(CN(C)C)c1]

General procedure E. 27% yield. The compound was converted to its 3HCl salt (white solid).

¹H NMR (free amine, CD₃OD, 300 Mz): δ 8.08-8.07 (m, 1H), 7.79-7.73 (m, 2H), 7.70-7.66 (m, 2H), 7.47-7.40 (m, 2H), 7.36-7.32 (m, 1H), 7.27-7.25 (m, 1H), 3.60 (s, 2H), 3.58 (s, 2H), 2.52 (br, 8H), 2.31-2.28 (m, 12H).

¹³C NMR (free amine, CD₃OD, 75 MHz): δ 152.8, 141.5, 140.5, 138.8, 135.1, 130.5, 130.1, 130.0, 129.6, 129.3, 128.3, 128.1, 120.7, 118.7, 117.6, 64.8, 63.5, 55.7, 53.5, 45.9, 45.2, 10.2.

MS (ESI) m/z 404 [M+H]⁺.

P_{HPLC}>98%. HPLC (C₄, 35 min): t_{R} 12.1 min, P_{HPLC} 98%; HPLC (C₁₈, 35 min): t_{R} 18.3 min, P_{HPLC} 98%.

### Example 23: N′-[[3-[3-[3-(dimethylaminomethyl)phenyl]-4-methyl-1H-pyrazol-1-yl]phenyl]methyl]-N,N,N′-trimethyl-propane-1,3-diamine (66)

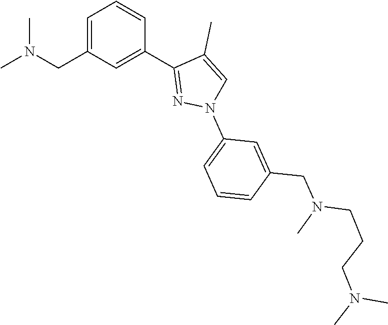

General procedure E. 78% yield. The compound was converted to its 3HCl salt (white solid).

¹H NMR (salt, CD₃OD, 300 Mz): δ 8.28-8.26 (m, 2H), 8.09 (s, 1H), 7.97-7.91 (m, 2H), 7.65-7.51 (m, 4H), 4.61-4.45 (m, 4H), 3.40-3.26 (m, 4H), 2.94 (s, 6H), 2.92 (s, 6H), 2.91 (s, 3H), 2.37 (br, 5H).

¹³C NMR (salt, CD₃OD, 75 MHz): δ 152.1, 141.9, 136.1, 132.3, 131.7, 131.6, 131.3, 131.1, 130.6, 130.2, 129.8, 129.6, 122.2, 120.9, 118.3, 62.1, 60.9, 55.6, 53.9, 43.6, 43.1, 40.2, 21.1, 10.4.

MS (ESI) m/z 420 [M+H]⁺.

P_{HPLC}>99%. HPLC (C₄, 35 min): t_{R} 12.8 min, P_{HPLC} 100%; HPLC (C₁₈, 35 min): t_{R} 17.9 min, P_{HPLC} 99%.

### Example 24: {[4-(1-{3-[(dimethylamino)methyl]phenyl}-4-methyl-1H-pyrazol-3-yl)phenyl]methyl}-[3-(dimethylamino)propyl]methylamine (67)

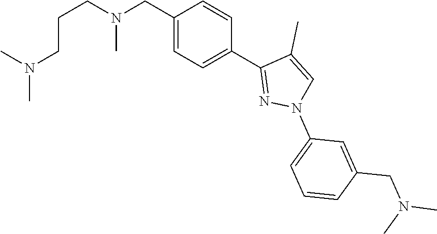

General procedure E. 63% yield. The compound was converted to its 3HCl salt (white solid).

¹H NMR (free amine, CD₃OD, 300 Mz): δ 8.05-8.05 (m, 1H), 7.75-7.67 (m, 4H), 7.44-7.39 (m, 3H), 7.23-7.21 (m, 1H), 3.54 (s, 2H), 3.51 (s, 2H), 2.41 (t, J=7.4 Hz, 2H), 2.36-2.31 (m, 2H), 2.29-2.22 (m, 18H), 1.77-1.67 (m, 2H).

¹³C NMR (free amine, CD₃OD, 75 MHz): δ 152.7, 141.4, 140.7, 138.9, 133.9, 130.6, 130.5, 129.2, 128.6, 128.2, 120.6, 118.7, 117.5, 64.7, 62.9, 58.6, 56.2, 45.4, 45.3, 42.4, 25.7, 10.4.

MS (ESI) m/z 420 [M+H]⁺.

P_{HPLC}>98%. HPLC (C₄, 30 min): t_{R} 12.6 min, P_{HPLC} 98%; HPLC (C₁₈, 30 min): t_{R} 16.6 min, P_{HPLC} 100%.

### Example 25: [3-(dimethylamino)propyl](methyl){[4-(4-methyl-1-{3-[(4-methylpiperazin-1-yl)methyl]phenyl}-1H-pyrazol-3-yl)phenyl]methyl}amine (68)

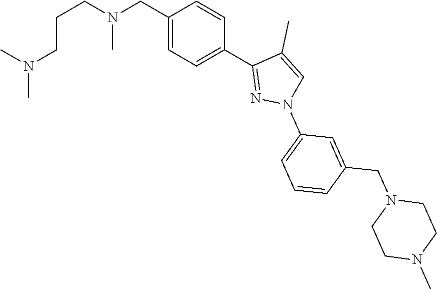 [SMILES: Cc1cn(-c2cccc(CN3CCN(C)CC3)c2)nc1-c1ccc(CN(C)CCCN(C)C)cc1]

General procedure E. 49% yield. The compound was converted to its 4HCl salt (white solid).

¹H NMR (free amine, CD₃OD, 300 Mz): δ 8.06-8.06 (m, 1H), 7.78-7.77 (m, 1H), 7.74-7.71 (m, 2H), 7.69-7.65 (m, 1H), 7.45-7.39 (m, 3H), 7.26-7.24 (m, 1H), 3.59 (s, 2H), 3.57 (s, 2H), 2.51 (br, 8H), 2.46-2.41 (m, 2H), 2.40-2.34 (m, 2H), 2.30-2.24 (m, 15H), 1.79-1.69 (m, 2H).

¹³C NMR (free amine, CD₃OD, 75 MHz): δ 152.8, 141.4, 140.4, 138.9, 134.0, 130.7, 130.5, 129.3, 128.7, 128.3, 120.7, 118.7, 117.6, 63.5, 62.9, 58.6, 56.2, 55.7, 53.6, 46.0, 45.3, 42.4, 25.6, 10.3.

MS (ESI) m/z 475 [M+H]⁺.

P_{HPLC}>99%. HPLC (C₄, 30 min): t_{R} 11.7 min, P_{HPLC} 100%; HPLC (C₁₈, 30 min): t_{R} 16.2 min, P_{HPLC} 99%.

### Example 27: [3-(dimethylamino)propyl][(4-{1-[3-({[3-(dimethylamino)propyl](methyl)amino}methyl)phenyl]-4-methyl-1H-pyrazol-3-yl}phenyl)methyl]methylamine (69)

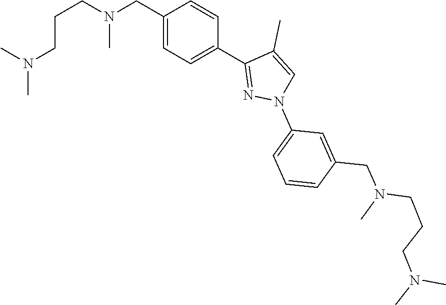

General procedure E. 59% yield. The compound was converted to its 4HCl salt (white solid).

¹H NMR (free amine, CD₃OD, 300 Mz): δ 8.06-8.06 (m, 1H), 7.77-7.76 (m, 1H), 7.73 (d, J=8.2 Hz, 2H), 7.68-7.64 (m, 1H), 7.44-7.38 (m, 3H), 7.25-7.22 (m, 1H), 3.57 (s, 2H), 3.55 (s, 2H), 2.42 (t, J=7.4 Hz, 4H), 2.37-2.29 (m, 7H), 2.24-2.22 (m, 18H), 1.78-1.67 (m, 4H).

¹³C NMR (free amine, CD₃OD, 75 MHz): δ 152.7, 141.4, 141.4, 139.0, 134.0, 130.6, 130.4, 129.2, 128.6, 128.1, 120.5, 118.5, 117.5, 62.9, 62.9, 58.6 (2C), 56.2 (2C), 45.4 (2C), 42.5, 42.4, 25.7, 25.7, 10.4.

MS (ESI) m/z 491 [M+H]⁺.

P_{HPLC}>98%. HPLC (C₄, 30 min): t_{R} 11.9 min, P_{HPLC} 98%; HPLC (C₁₈, 30 min): t_{R} 15.9 min, P_{HPLC} 98%.

### Example 27: N′-[[3-[1-[3-(dimethylaminomethyl)phenyl]-4-methyl-1H-pyrazol-3-yl]phenyl]methyl]-N,N,N′-trimethyl-propane-1,3-diamine (70)

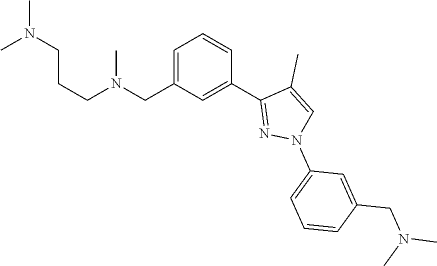

General procedure E. 84% yield. The compound was converted to its 3HCl salt (white solid).

¹H NMR (free amine, CD₃OD, 300 Mz): δ 8.05 (s, 1H), 7.76-7.73 (m, 2H), 7.71-7.64 (m, 2H), 7.44-7.39 (m, 2H), 7.40 (ddd, J=7.7, 1.3, 1.3 Hz, 1H), 7.23-7.21 (m, 1H), 3.57 (s, 2H), 3.51 (s, 2H), 2.46-2.35 (m, 4H), 2.29-2.24 (m, 18H), 1.78-1.68 (m, 2H).

¹³C NMR (free amine, CD₃OD, 75 MHz): δ 152.8, 141.4, 140.7, 139.7, 135.0, 130.5, 129.9, 129.7, 129.5, 129.2, 128.2, 127.7, 120.6, 118.7, 117.6, 64.7, 63.1, 58.6, 56.2, 45.3 (2C), 42.4, 25.5, 10.4.

MS (ESI) m/z 420 [M+H]⁺.

P_{HPLC}>96%. HPLC (C₄, 35 min): t_{R} 7.9 min, P_{HPLC} 99%; HPLC (C₁₈, 35 min): t_{R} 12.7 min, P_{HPLC} 96%.

### Example 28: N,N,N′-trimethyl-N′-[[3-[4-methyl-1-[3-[(4-methylpiperazin-1-yl)methyl]phenyl]pyrazol-3-yl]phenyl]methyl]propane-1,3-diamine (71)

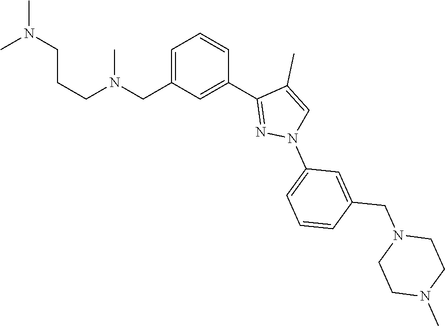 [SMILES: Cc1cn(-c2cccc(CN3CCN(C)CC3)c2)nc1-c1cccc(CN(C)CCCN(C)C)c1]

General procedure E. 89% yield. The compound was converted to its 4HCl salt (white solid).

¹H NMR (free amine, CD₃OD, 300 Mz): δ 8.05 (s, 1H), 7.78-7.73 (m, 2H), 7.69-7.64 (m, 2H), 7.44-7.38 (m, 2H), 7.33-7.31 (m, 1H), 7.25-7.22 (m, 1H), 3.57 (s, 2H), 3.56 (s, 2H), 2.49 (br, 8H), 2.45-2.33 (m, 4H), 2.29-2.23 (m, 15H), 1.77-1.67 (m, 2H).

¹³C NMR (free amine, CD₃OD, 75 MHz): δ 152.8, 141.4, 140.5, 139.7, 135.0, 130.4, 129.9, 129.7, 129.5, 129.2, 128.2, 127.7, 120.6, 118.7, 117.6, 63.5, 63.1, 58.6, 56.2, 55.7, 53.6, 46.0, 45.4, 42.5, 25.6, 10.4.

MS (ESI) m/z 475 [M+H]⁺.

P_{HPLC}>99%. HPLC (C₄, 35 min): t_{R} 4.5 min, P_{HPLC} 100%; HPLC (C₁₈, 35 min): t_{R} 12.5 min, P_{HPLC} 99%.

### Example 29: N′-[[3-[1-[3-[[3-(dimethylamino)propyl-methyl-amino]methyl]phenyl]-4-methyl-pyrazol-3-yl]phenyl]methyl]-N,N,N′-trimethyl-propane-1,3-diamine (72)

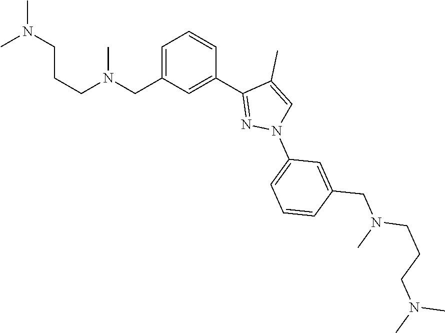

General procedure E. 66% yield. The compound was converted to its 4HCl salt (white solid).

¹H NMR (free amine, CD₃OD, 300 Mz): δ 8.07 (s, 1H), 7.78-7.73 (m, 2H), 7.69-7.65 (m, 2H), 7.42 (dd, J=7.7, 7.7 Hz, 2H), 7.35-7.32 (m, 1H), 7.26-7.24 (m, 1H), 3.59 (s, 2H), 3.58 (s, 2H), 2.47-2.41 (m, 4H), 2.39-2.34 (m, 4H), 2.3-2.24 (m, 21H), 1.79-1.69 (m, 4H).

¹³C NMR (free amine, CD₃OD, 75 MHz): δ 152.9, 141.4, 139.7, 135.0, 130.4, 129.9, 129.8, 129.5, 129.2, 128.1, 127.7, 120.6, 118.6, 117.6, 63.1, 62.9, 58.6 (2C), 56.3, 56.2, 45.4 (2C), 42.5 (2C), 25.7 (2C), 10.4.

MS (ESI) m/z 491 [M+H]⁺.

P_{HPLC}>98%. HPLC (C₄, 35 min): t_{R} 7.7 min, P_{HPLC} 99%; HPLC (C₁₈, 35 min): t_{R} 12.1 min, P_{HPLC} 98%.

### Biological Evaluations

### Materials and Methods

### 1. Cytotoxicity Assays

Cytotoxicity was measured using 3-(4,5-dimethylthiazol-2-yl)-5-(3-carboxymethoxyphenyl)-2-(4-sulfophenyl)-2H-tetrazolium (MTS) tests. SY5Y-APP^{695WT} cells were plated in a 96-wells plate at 3.10⁴ cells per well and allowed to attach for 24 h. Cells were then incubated for 72 h with 100 μL of DMEM medium with 10% SVF containing (or not) the defined concentration of drugs. Cytotoxicity was determined by using the colorimetric MTS assay (Cell Titer 96® Aqueous One Solution Cell Proliferation Assay-MTS Promega) according to the manufacturer's instructions. Absorbance was read at 490 nm.

### 2. Cell Culture and Treatments

The human neuroblastoma cell line SY5Y-APP^{695WT} was maintained in Dulbecco's modified Eagle medium (DMEM, high glucose, pyruvate—GIBCO, Life Technologies) supplemented with 10% fetal bovine serum, 2 mM L-glutamine, 1 mM non-essential amino acids and penicillin/streptomycin (GIBCO, Life Technologies) at 37°C in a 5% CO₂ humidified incubator [36]. For compound treatment, a 10 mM stock solution was diluted in freshly supplemented DMEM medium to obtain the precise final concentration of the drug. Cells were plated at a density of 5.10⁵ cells per well into 12-well plates and cultured with 1 mL supplemented DMEM cell medium for 24 h before compound exposure. The following day, the cell medium was replaced with fresh medium containing the compounds diluted at the indicated concentrations. Cells were treated for 24 h. At the end of treatments, the cell medium was collected and kept at −80°C until use, cells were rinsed once with PBS and extracted in 100 μL of Laemmli buffer (10 mM Tris, 20% glycerol, and 2% sodium dodecyl sulfate) using a cell-scraper. The cell lysate was further sonicated (30 pulses of 0.5 s, 60 Hz) for 5 min. Total protein concentration was determined using the Pierce BCA Protein Assay Kit (Thermo Scientific) according to the manufacturer's instructions. Samples were stored at −80°C until analysis.

### 3. Western Blot Analysis

Cell protein lysates were prepared for western-blot analysis by diluting the sample with 1 volume of NuPAGE® lithium dodecyl sulfate (LDS) 2× sample buffer supplemented with 20% NuPAGE® sample reducing agents (Invitrogen). Samples were heated for 10 min at 100°C Ten μg of total proteins per well were loaded onto precast 4-12% Criterion XT Bis-Tris polyacrylamide gels (Bio-Rad) and electrophoresis was achieved after applying a tension of 150 V during 90 min using a Criterion electrophoresis Cell with the NuPAGE® MOPS SDS running buffer (1×). Proteins were transferred to a nitrocellulose membrane of 0.45 μM pore size (G&E Healthcare) using the Criterion blotting system and applying a tension of 100 V for 45 min. To resolve proteins of low molecular weights such as carboxy-terminal fragments of APP, 12% Criterion XT Bis-Tris polyacrylamide gels (Bio-Rad) were used and electrophoresis was performed during 70 min at 150 V in a NuPAGE® MES SDS running buffer (1×). Proteins were transferred to a nitrocellulose membrane of 0.2 μm pore size (G&E Healthcare) at 100 V for 40 min. Molecular weight calibration was achieved using molecular weight markers (Novex and Magic Marks, Life Technologies). Protein transfer and quality were determined by a reversible Ponceau Red coloration (0.2% xylidine Ponceau Red and 3% trichloroacetic acid). Membranes were then blocked in 25 mM Tris-HCl pH 8.0, 150 mM NaCl, 0.1% Tween-20 (v/v) (TNT) and 5% (w/v) of skimmed milk or 5% (w/v) of bovine serum albumin depending on the antibody during 1 h. The membrane was rinsed three times at 10 min in TNT before incubation with the primary antibody overnight at 4°C. Membrane was incubated with the secondary antibody for 45 min at rt. The immunoreactive complexes were revealed using the ECL™ Western Blotting Detection Reagents (G&E Healthcare) and image acquisitions were performed with the Amersham Imager 600 (G&E Healthcare). Quantifications of protein expression levels were performed with Image Quant TL (G&E Healthcare).

### 4. Antibodies

Primary antibodies used in this study for western-blot analysis included a well-characterized homemade rabbit antiserum against the last 17 amino acids of APP, named APP-Cter-C17 (1/5000), LC3B obtained from Cell Signaling (1/1000), p62 (Abcam, 1/2000) and α-tubulin (Sigma, 1/10000). The anti-histone H3 (1/10 000) used for normalization was obtained from Sigma. Secondary antibodies (peroxidase-labeled goat anti-rabbit IgG, 1/5000 or peroxidase-labeled horse anti-mouse IgG, 1/50000) were obtained from Vector Laboratories.

5. Quantification of Secreted Aβ and sAPP

Conditioned media of SYSY-APP^{695WT} collected at the end of treatments were centrifuged at 1000×g for 5 min to eliminate cell debris.

Aβ₁₋₄₀/Aβ_{1_42} peptides, Aβₓ₋₃₈/Aβₓ₋₄₀/Aβₓ₋₄₂ and sAPPα/sAPPβ concentrations in μg/mL were determined, respectively, using amyloid-beta 40 and 42 Human ELISA kits (Invitrogen), V-PLEX Plus Aβ Peptide Panel 1 (4G8) Kit (Meso Scale Diagnostics, MSD R©) and sAPPα/sAPPβ multiplex kit (Meso Scale Diagnostics, MSD R©) according to the manufacturer's instructions.

### Results

### Effect of Compounds on APP Metabolism

Compounds of the invention (compounds 16-17, 24-35, 57-58 and 61-72) were evaluated for their ability to modulate APP processing on SYSY human neuroblastoma cell line stably expressing the neuronal isoform of human wild-type APP695 (SY5Y-APP^{wt}). APP carboxy-terminal fragments (αCTFs and AICD) levels were assessed by western blotting (WB). The effect of reference and test compounds on αCTFs and AICD is expressed as the intensity of corresponding WB bands. The concentration of compounds able to increase 20-times αCTFs and AICD (C₂₀) when compared to the control condition was calculated. Selected compounds were also evaluated for their ability to modulate the secretion of soluble APP fragments (sAPPα and sAPPβ). Aβ₁₋ₓ levels (Aβ_{1_40} and Aβ₁₋₄₂) after treatment with reference and tested compounds were measured in the cell media by ELISA (Table 2). Results are expressed as IC₅₀ values which correspond to the concentration of a given compound that inhibits Aβ₁₋ₓ concentration by 50% (either A31_40 or Aβ₁₋₄₂) in comparison to the concentration of Aβ₁₋ₓ in non-treated SY5Y-APP^{WT} cells. Selected hit compounds were then further evaluated for their ability to decrease the level of N-truncated Aβ peptides (Aβₓ₋₃₈, Aβₓ₋₄₀, and Aβₓ₋₄₂). Results obtained for the compounds of the invention are compared with results obtained for compounds of the prior art: CQ: chloroquine; A1: compound 29 of P. Melnyk et al., ACS Chem. Neurosci. 2015, 6, 559-569; B1: compound 8 of M. Gay et al., Bioorg. Med. Chem. 2018, 26, 2151-2164.

TABLE 2
Effects of compounds of the invention on the metabolism of APP

| | Aβ₁₋₄₀ | Aβ₁₋₄₂ | αCTF | AICD | Cytotoxicity |
|---|---|---|---|---|---|
| Compound | IC₅₀ (μM)^{a} | IC₅₀ (μM)^{a} | (C₂₀, μM)^{b, c} | (C₂₀, μM)^{b, c} | CC₅₀ (μM)^{d} |
| CQ^{e} | 7.0 | 12.7 | >>10 | >>10 | 30 |
| A1^{f} | 1.7 ± 0.2 | 2.1 ± 0.5 | >10 | 5.0 | 30 |
| B1^{f} | 5.1 ± 0.1 | 11.2 ± 4.2 | >10 | 9.5 | 30 |
| 16 | 6.2 ± 0.6 | 6.5 ± 1.1 | 3.6 | 4.6 | 23 |
| 17 | 4.8 ± 1.2 | 5.4 ± 2.4 | 2.0 | 3.6 | 34 |
| 24 | 1.5 ± 0.1 | 2.5 ± 0.2 | 1.6 | 2.2 | >100 |
| 25 | 2.9 ± 0.1 | 3.2 ± 1.0 | 1.0 | 1.2 | >100 |
| 26 | 1.6 ± 0.1 | 2.4 ± 0.7 | 1.3 | 1.8 | >100 |
| 27 | 2.6 ± 0.1 | 3.8 ± 0.4 | 1.3 | 1.6 | >100 |
| 28 | 1.9 ± 0.1 | 2.4 ± 0.7 | 1.2 | 1.7 | >100 |
| 29 | 3.4 ± 0.1 | 5.0 ± 0.1 | 1.0 | 2.0 | >100 |
| 30 | 2.2 ± 0.3 | 3.8 ± 0.1 | 1.2 | 1.6 | >100 |
| 31 | 4.0 ± 0.6 | 5.8 ± 0.6 | 1.0 | 1.5 | >100 |
| 32 | 4.2 ± 0.1 | 6.1 ± 0.3 | 1.1 | 1.8 | >100 |
| 33 | 9.2 ± 0.4 | 9.7 ± 1.2 | 6.1 | 8.9 | >100 |
| 34 | 5.7 ± 0.5 | 8.3 ± 0.2 | 1.1 | 1.9 | >100 |
| 35 | 12.4 ± 0.6 | 13.2 ± 0.2 | 5.1 | 5.4 | >100 |
| 61 | 3.9 ± 0.3 | 4.1 ± 2.1 | 2.7 | 2.7 | 27 |
| 62 | 2.4 ± 0.2 | 3.2 ± 0.8 | 2.0 | 3.5 | 11 |
| 63 | 2.0 ± 0.2 | 2.7 ± 0.2 | 1.2 | 1.5 | 13 |
| 64 | 3.8 ± 0.2 | 4.1 ± 1.6 | 3.8 | 4.9 | >100 |
| 65 | 2.0 ± 0.7 | 2.1 ± 0.4 | 1.7 | 2.8 | >100 |
| 66 | 2.2 ± 0.3 | 2.5 ± 1.0 | 2.2 | 2.4 | 47 |
| 57 | 2.9 ± 1.0 | 3.5 ± 0.8 | >>10 | 8.5 | >100 |
| 67 | 1.3 ± 0.1 | 2.1 ± 0.5 | 1.4 | 1.2 | 12 |
| 68 | 1.2 ± 0.1 | 1.6 ± 0.1 | 1.0 | 1.0 | 6 |
| 69 | 1.4 ± 0.1 | 1.6 ± 0.1 | 0.6 | 1.0 | 2 |
| 58 | 3.3 ± 1.3 | 3.5 ± 0.7 | >>10 | 7.9 | 20 |
| 70 | 1.5 ± 0.1 | 2.1 ± 0.1 | 1.3 | 1.7 | 51 |
| 71 | 1.2 ± 0.1 | 1.7 ± 0.1 | 0.7 | 1.0 | 25 |
| 72 | 2.6 ± 0.2 | 3.3 ± 0.1 | 0.6 | 0.8 | 26 |

^{a}Compound concentration inhibiting 50% of Aβ1-40 or Aβ1-42 peptide secretion in SY5Y cells. IC₅₀ values are expressed as mean ± SD of at least two experiments performed in triplicate.
^{b}Mean values calculated based on at least three independent experiments with less than 10% deviation.
^{c}C₂₀ indicates the concentration of compound necessary to increase by 20-fold the expression of APP fragments compared to untreated control conditions.
^{d}Compound concentration causing 50% of cell death after 72 h treatment.
^{e}P. Melnyk et al., ACS Chem. Neurosci. 2015, 6, 559-569.
^{f}M. Gay et al., Eur. J. Med. Chem. 2018, 159, 104-125.
nd: not determined.

Results show that compounds of the invention were able to decrease Aβ₁₋ₓ secretion.

The activity of compounds of the invention on sAPPα 6 and sAPPβ was further explored. The expression of sAPPβ and sAPPα, which are by-products of β- and α-secretase respectively, were quantified in media of treated cells. All compounds were shown to decrease sAPPβ efficiently (Table 3).

TABLE 3
Effects of the compounds of the invention
on the metabolism of APP

| | sAPPβ | sAPPα |
|---|---|---|
| Compound | IC₅₀ (μM)^{a} | C_{1.5} (μM)^{b} |
| 33 | 6.8 ± 0.3 | >10 |
| 34 | 4.5 ± 0.7 | >10 |
| 67 | 3.2 ± 1.4 | 1.4 |
| 69 | 2.8 ± 1.1 | >5 |
| 71 | 4.0 ± 0.4 | 1.1 |

^{a}Compound concentration inhibiting 50% of sAPPβ secretion in SY5Y cells. IC₅₀ values are expressed as mean ± SD of at least three experiments performed in triplicate.
^{b}C_{1.5} indicates the concentration of compound necessary to increase by 1.5-fold the amount of sAPPα fragments compared to untreated control conditions.

These results strongly suggest that the compounds of the invention, in particular compounds 67 and 71, reduce Aβ₁₋ₓ peptides secretion by repressing the β-secretase cleavage of APP while increasing the secretion of sAPPα.

### Effect of Compounds on Autophagic Flux and Lysosomal Degradation Pathways

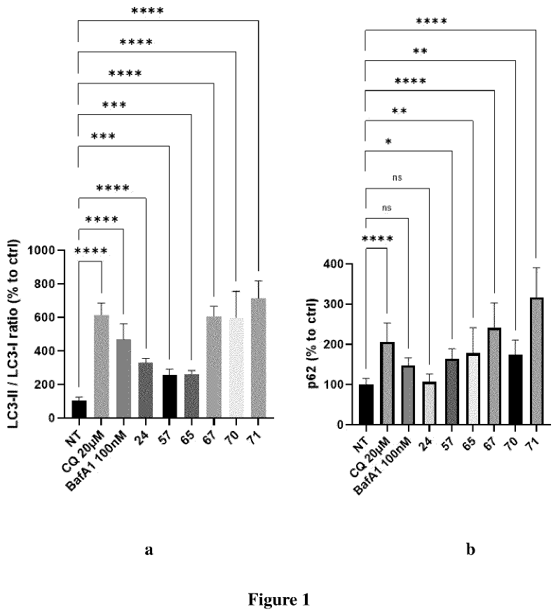

The effect of compounds of the invention on the autophagic flux was studied, in particular, the effect of compounds 24, 57, 65, 67, 70 and 71 on two markers associated with autophagy: p62 expression and LC3B-I lipidation into LC3B-II, was evaluated. In addition to CQ, Bafilomycin A1, a well-known inhibitor of autophagic flux, was used as a control. The results are shown in FIG. 1.

The results show that the compounds of the invention have a β-secretase modulatory activity, low toxicity, and are able to modulate autophagy markers LC3B II/I ratio and p62, which shows that the compounds of the invention are active against both pathophysiological processes of AD.

1. A compound of Formula I: 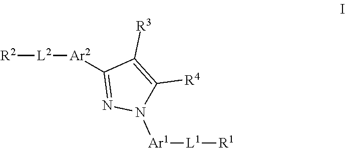
and pharmaceutically acceptable salts and solvates thereof,
wherein
Ar¹ is phenyl optionally substituted by one or more substituent(s) selected from the group consisting of halo, C1-C3-alkyl, C1-C3-haloalkyl and C1-C3-alkoxy;
L¹ is C1-C4-alkylene optionally substituted by one or more substituent(s) selected from the group consisting of halo and C1-C3-alkyl;
R¹ is selected from the group consisting of —OH, —N(C1-C6-alkyl)₂, 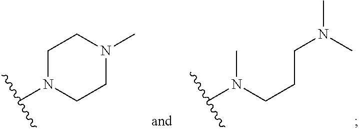
Ar² is phenyl optionally substituted by one or more substituent(s) selected from the group consisting of halo, C1-C3-alkyl, C1-C3-haloalkyl and C1-C3-alkoxy;
L² is C1-C4-alkylene optionally substituted by one or more substituent(s) selected from the group consisting of halo and C1-C3-alkyl;
R² is selected from the group consisting of —N(C1-C6-alkyl)₂, 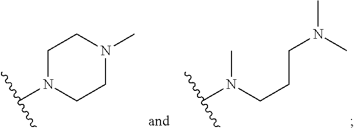
R³ is C1-C4-alkyl or —(CH₂)ₙ—NMe₂ wherein n is an integer from 1 to 3; and
R⁴ is H, halo or C1-C4-alkyl.

2. The compound according to claim 1, wherein Ar¹ and Ar² are phenyl.

3. The compound according to claim 1, wherein L¹ is CH₂.

4. The compound according to claim 1, wherein L² is C1-C3-alkylene.

5. The compound according to claim 1, wherein R⁴ is H.

6. The compound according to claim 1, having Formula II: 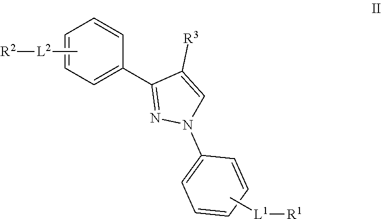
and pharmaceutically acceptable salts and solvates thereof,
wherein
L¹, R¹, L², R² and R³ are as defined in claim 1.

7. The compound according to claim 1, having Formula III: 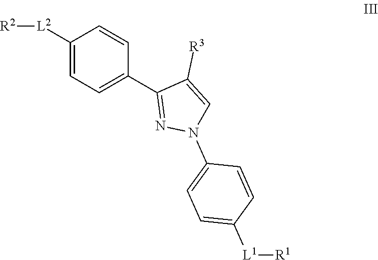
and pharmaceutically acceptable salts and solvates thereof,
wherein
L¹, R¹, L², R² and R³ are as defined in claim 1.

8. The compound according to claim 1, having Formula IV: 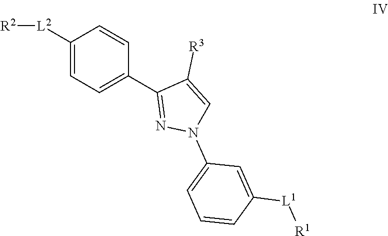
and pharmaceutically acceptable salts and solvates thereof,
wherein
L¹, R¹, L², R² and R³ are as defined in claim 1.

9. The compound according to claim 1, having Formula V: 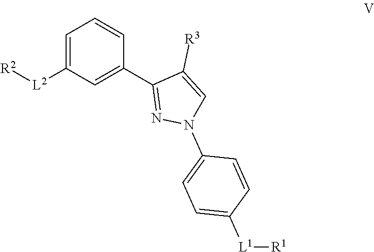
and pharmaceutically acceptable salts and solvates thereof,
wherein
L¹, R¹, L², R² and R³ are as defined in claim 1.

10. The compound according to claim 1, having Formula VI: 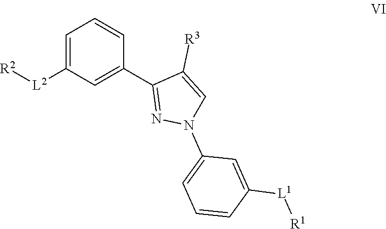
and pharmaceutically acceptable salts and solvates thereof,
wherein
L¹, R¹, L², R² and R³ are as defined in claim 1.

11. The compound according to claim 1, selected from the group consisting of:
[4-[4-(dimethylaminomethyl)-3-[4-[3-(dimethylamino)propyl]phenyl]-1H-pyrazol-1-yl]phenyl]methanol;
[4-[4-[3-(dimethylamino)propyl]-3-[4-[3-(dimethylamino)propyl]phenyl]-1H-pyrazol-1-yl]phenyl]methanol;
3-[4-[4-(dimethylaminomethyl)-1-[4-(dimethylaminomethyl)phenyl]-1H-pyrazol-3-yl]phenyl]-N,N-dimethyl-propan-1-amine;
3-[4-[1-[4-(dimethylaminomethyl)phenyl]-4-[3-(dimethylamino)propyl]-1H-pyrazol-3-yl]phenyl]-N,N-dimethyl-propan-1-amine;
3-[4-[4-(dimethylaminomethyl)-1-[3-(dimethylaminomethyl)phenyl]-1H-pyrazol-3-yl]phenyl]-N,N-dimethyl-propan-1-amine;
3-[4-[1-[3-(dimethylaminomethyl)phenyl]-4-[3-(dimethylamino)propyl]-1H-pyrazol-3-yl]phenyl]-N,N-dimethyl-propan-1-amine;
3-[4-[4-(dimethylaminomethyl)-1-[4-[(4-methylpiperazin-1-yl)methyl]phenyl]-1H-pyrazol-3-yl]phenyl]-N,N-dimethyl-propan-1-amine;
3-[4-[4-[3-(dimethylamino)propyl]-1-[4-[(4-methylpiperazin-1-yl)methyl]phenyl]-1H-pyrazol-3-yl]phenyl]-N,N-dimethyl-propan-1-amine;
3-[4-[4-(dimethylaminomethyl)-1-[3-[(4-methylpiperazin-1-yl)methyl]phenyl]-1H-pyrazol-3-yl]phenyl]-N,N-dimethyl-propan-1-amine;
3-[4-[4-[3-(dimethylamino)propyl]-1-[3-[(4-methylpiperazin-1-yl)methyl]phenyl]-1H-pyrazol-3-yl]phenyl]-N,N-dimethyl-propan-1-amine;
N′-[[4-[4-(dimethylaminomethyl)-3-[4-[3-(dimethylamino)propyl]phenyl]-1H-pyrazol-1-yl]phenyl]methyl]-N,N,N′-trimethyl-propane-1,3-diamine;
N′-[[4-[4-[3-(dimethylamino)propyl]-3-[4-[3-(dimethylamino)propyl]phenyl]-1Hpyrazol-1-yl]phenyl]methyl]-N,N,N′-trimethyl-propane-1,3-diamine;
N′-[[3-[4-(dimethylaminomethyl)-3-[4-[3-(dimethylamino)propyl]phenyl]-1H-pyrazol-1-yl]phenyl]methyl]-N,N,N′-trimethyl-propane-1,3-diamine;
N′-[[3-[4-[3-(dimethylamino)propyl]-3-[4-[3-(dimethylamino)propyl]phenyl]-1H-pyrazol-1-yl]phenyl]methyl]-N,N,N′-trimethyl-propane-1,3-diamine;
[3-[3-[4-(dimethylaminomethyl)phenyl]-4-methyl-1H-pyrazol-1-yl]phenyl]methanol;
(3-{3-[4-({[3-(dimethylamino)propyl](methyl)amino}methyl)phenyl]-4-methyl-1H-pyrazol-1-yl}phenyl)methanol;
[3-[3-[3-[[(3-(dimethylamino)propyl)(methyl)amino]methyl]phenyl]-4-methyl-1H-pyrazol-1-yl]phenyl]methanol;
{[4-(1-{3-[(dimethylamino)methyl]phenyl}-4-methyl-1H-pyrazol-3-yl)phenyl]methyl}dimethylamine;
dimethyl({[4-(4-methyl-1-{3-[(4-methylpiperazin-1-yl)methyl]phenyl}-1H-pyrazol-3-yl)phenyl]methyl})amine;
[(4-{1-[3-({[3-(dimethylamino)propyl](methyl)amino}methyl)phenyl]-4-methyl-1H-pyrazol-3-yl}phenyl)methyl]dimethylamine;
1-[3-[1-[3-(dimethylaminomethyl)phenyl]-4-methyl-1H-pyrazol-3-yl]phenyl]-N,N-dimethylmethanamine;
N,N-dimethyl-1-[3-[4-methyl-1-[3-[(4-methylpiperazin-1-yl)methyl]phenyl]-1H-pyrazol-3-yl]phenyl]methanamine;
N′-[[3-[3-[3-(dimethylaminomethyl)phenyl]-4-methyl-1H-pyrazol-1-yl]phenyl]methyl]-N,N,N′-trimethyl-propane-1,3-diamine;
{[4-(1-{3-[(dimethylamino)methyl]phenyl}-4-methyl-1H-pyrazol-3-yl)phenyl]methyl}-[3-(dimethylamino)propyl]methylamine;
[3-(dimethylamino)propyl](methyl){[4-(4-methyl-1-{3-[(4-methylpiperazin-1-yl)methyl]phenyl}-1H-pyrazol-3-yl)phenyl]methyl}amine;
[3-(dimethylamino)propyl][(4-{1-[3-({[3-(dimethylamino)propyl](methyl)amino}methyl)phenyl]-4-methyl-1H-pyrazol-3-yl}phenyl)methyl]methylamine;
N′-[[3-[1-[3-(dimethylaminomethyl)phenyl]-4-methyl-1H-pyrazol-3-yl]phenyl]methyl]-N,N,N′-trimethyl-propane-1,3-diamine;
N,N,N′-trimethyl-N′-[[3-[4-methyl-1-[3-[(4-methylpiperazin-1-yl)methyl]phenyl]-1H-pyrazol-3-yl]phenyl]methyl]propane-1,3-diamine; and
N′-[[3-[1-[3-[[3-(dimethylamino)propyl-methyl-amino]methyl]phenyl]-4-methyl-1H-pyrazol-3-yl]phenyl]methyl]-N,N,N′-trimethyl-propane-1,3-diamine.

12. A pharmaceutical composition comprising a compound according to claim 1, or a pharmaceutically acceptable salt or solvate thereof, and at least one pharmaceutically acceptable carrier, diluent, excipient and/or adjuvant.

13. (canceled)

14. A method of treating and/or preventing a disease involving the formation of amyloid plaques and/or in which dysfunction of the amyloid precursor protein (APP) metabolism occurs in a subject in need thereof, comprising the step of administering to the subject an effective amount of a compound according to claim 1, or a pharmaceutically acceptable salt or solvate thereof.

15. The method according to claim 14, wherein the disease involving the formation of amyloid plaques and/or in which dysfunction of the APP metabolism occurs is selected from the group consisting of Alzheimer's disease, Lewy body disease, Down syndrome, amyloid angiopathy, Parkinson's disease, Creutzfeldt-Jakob disease, amyotrophic lateral sclerosis and frontotemporal degeneration.

16. The method according to claim 14, wherein the disease involving the formation of amyloid plaques and/or in which dysfunction of the APP metabolism occurs is Alzheimer's disease.
